(19) United States
(12) Patent Application Publication
(10) Pub. No.: US 2007/0042354 A1
(43) Pub. Date: Feb. 22, 2007

(54) Detecting pathogens in companion animals

(76) Inventor: Eric K. Engelhard, Davis, CA (US)

Correspondence Address: FISH & RICHARDSON P.C., PO BOX 1022, MINNEAPOLIS, MN, 55440-1022, US

(21) Appl. No.: 11/207,316

(22) Filed: Aug. 19, 2005

(51) Int. Cl. C12Q 1/70; C12Q 1/68; C07H 21/04; C12M 3/00

(52) U.S. Cl. 435/5; 435/6; 435/287.2; 536/24.1

(57) ABSTRACT

This document relates to methods and materials involved in determining whether or not an animal contains a pathogen. For example, nucleic acid primer pairs, combinations of nucleic acid primer pairs, nucleic acid arrays (e.g., diagnostic cards) containing nucleic acid primer pairs or combinations of nucleic acid primer pairs, methods for making such nucleic acid arrays, and methods for diagnosing animals infected with a pathogen are provided.

### STATEMENT AS TO FEDERALLY SPONSORED RESEARCH

Funding for the work described herein was provided by the federal government, which may have certain rights in the invention.

### BACKGROUND

1. Technical Field

This document relates to methods and materials involved in detecting pathogens (e.g., bacteria, fungi, parasites such as eukaryote parasites, or viruses) in companion animals (e.g., dogs or cats).

2. Background Information

Like other animals, companion animals such as dogs and cats can become infected with pathogens. In some cases, an infected companion animal can become ill and even die because of the infection. For example, dogs infected with Canine parvovirus can die if untreated. Properly diagnosing an infected companion animal can allow veterinarians to treat the infected animal, thereby leading to a potentially longer and healthier life for the companion animal.

### SUMMARY

This document relates to methods and materials involved in detecting pathogens in animals such as companion animals. For example, this document provides nucleic acid primer pairs that can be used in an amplification reaction to detect the presence or absence of a pathogen's nucleic acid within a sample obtained from the animal being tested. This document also provides combinations of nucleic acid primer pairs, nucleic acid arrays (e.g., diagnostic cards) containing nucleic acid primer pairs or combinations of nucleic acid primer pairs, methods for making such nucleic acid arrays, and methods for diagnosing animals infected with a pathogen. Such methods and materials can allow, for example, veterinarians to diagnose an animal as having a particular infection. For example, the nucleic acid primer pairs provided herein can allow a veterinarian to diagnose a cat as having a feline immunodeficiency virus or as being free of a feline immunodeficiency virus. Once diagnosed as having a particular infection, a veterinarian can identify proper treatments or procedures for the infected animal.

The description provided herein is based, in part, on the discovery of nucleic acid primer pairs having the ability to not only amplify particular nucleic acid sequences from particular pathogens, but also to not amplify nucleic acid sequences from non-pathogen sources such as the host's genome. The description provided herein also is based, in part, on the discovery of sets of nucleic acid primer pairs that can be used simultaneously under the same amplification reaction conditions to amplify different target nucleic acids if present in the sample being tested. For example, a single diagnostic card having ten separate microfluidic chambers, each of which contains a different primer pair provided herein, can be used in a single amplification reaction to detect the presence or absence of up to ten different pathogens. Having the ability to test for the presence or absence of multiple pathogens using a single diagnostic card and a single amplification reaction can allow veterinarians to diagnose an animal's condition rapidly in a cost effective manner.

In general, this document features a composition comprising, or consisting essentially of, a mixture, wherein the mixture comprises at least one primer pair selected from the group consisting of primer pair numbers 951, 1001, 151, 701, 1051, 351, 801, 101, and 1101, wherein primer pair number 951 amplifies a sequence present in a feline immunodeficiency virus, wherein primer pair number 1001 amplifies a sequence present in a feline leukemia virus, wherein primer pair number 151 amplifies a sequence present in a Borrelia burgdorferi organism, wherein primer pair number 701 amplifies a sequence present in a Dirofilaria immitis organism, wherein primer pair number 1051 amplifies a sequence present in a feline parvovirus, wherein primer pair number 351 amplifies a sequence present in a canine distemper virus, wherein primer pair number 801 amplifies a sequence present in a Ehrlichia canis organism, wherein primer pair number 101 amplifies a sequence present in a Bordetella organism, and wherein primer pair number 1101 amplifies a sequence present in a Giardia organism. The mixture can be a solid. The mixture can be a liquid. The mixture can comprise primer pair number 951. The mixture can comprise primer pair number 1001. The mixture can comprise primer pair number 151.

In another embodiment, this document features an article of manufacture comprising, or consisting essentially of: (a) a substrate defining a microfluidic chamber and (b) a mixture comprising at least one primer pair selected from the group consisting of primer pair numbers 951, 1001, 151, 701, 1051,351, 801, 101, and 1101; wherein the mixture is within the chamber; wherein primer pair number 951 is capable of amplifying, within the chamber, a sequence present in a feline immunodeficiency virus; wherein primer pair number 1001 is capable of amplifying, within the chamber, a sequence present in a feline leukemia virus; wherein primer pair number 151 is capable of amplifying, within the chamber, a sequence present in a Borrelia burgdorferi organism; wherein primer pair number 701 is capable of amplifying, within the chamber, a sequence present in a Dirofilaria immitis organism; wherein primer pair number 1051 is capable of amplifying, within the chamber, a sequence present in a feline parvovirus; wherein primer pair number 351 is capable of amplifying, within the chamber, a sequence present in a canine distemper virus; wherein primer pair number 801 is capable of amplifying, within the chamber, a sequence present in a Ehrlichia canis organism; wherein primer pair number 101 is capable of amplifying, within the chamber, a sequence present in a Bordetella organism; and wherein primer pair number 1101 is capable of amplifying, within the chamber, a sequence present in a Giardia organism. The mixture can be a solid. The mixture can be a liquid. The mixture can comprise primer pair number 951. The mixture can comprise primer pair number 1001. The mixture can comprise primer pair number 151.

In another aspect, this document features a diagnostic card for determining whether or not a cat contains any pathogen selected from the group consisting of feline immunodeficiency virus, feline leukemia virus, and Borrelia burgdorferi; wherein the card comprises, or consists essentially of, a plurality of microfluidic chambers; wherein at least one of the microfluidic chambers comprises the primers of primer pair number 951, which is capable of amplifying, within the chamber, a sequence present in a feline immunodeficiency virus; wherein at least one of the microfluidic chambers comprises the primers of primer pair number 1001, which is capable of amplifying, within the chamber, a sequence present in a feline leukemia virus; and wherein at least one of the microfluidic chambers comprises the primers of primer pair number 151, which is capable of amplifying, within the chamber, a sequence present in a Borrelia burgdorferi organism.

In another aspect, this document features a diagnostic card for determining whether or not a dog contains any pathogen selected from the group consisting of Borrelia burgdorferi, Dirofilaria immitis, and canine distemper virus; wherein the card comprises, or consists essentially of, a plurality of microfluidic chambers; wherein at least one of the microfluidic chambers comprises the primers of primer pair number 151, which is capable of amplifying, within the chamber, a sequence present in a Borrelia burgdorferi organism; wherein at least one of the microfluidic chambers comprises the primers of primer pair number 701, which is capable of amplifying, within the chamber, a sequence present in a Dirofilaria immitis organism; and wherein at least one of the microfluidic chambers comprises the primers of primer pair number 351, which is capable of amplifying, within the chamber, a sequence present in a canine distemper virus.

In another aspect, this document features a method for determining whether or not a mammal contains a pathogen, wherein the method comprises, or consists essentially of, performing an amplification reaction with a primer pair selected from the group consisting of primer pair numbers 951, 1001, 151, 701, 1051, 351, 801, 101, and 1101 to determine whether or not a sample from the mammal contains nucleic acid capable of being amplified with the primer pair, wherein the presence of the nucleic acid indicates that the mammal contains the pathogen. The mammal can be a cat. The mammal can be a dog. The sample can be a blood sample. The primer pair can be primer pair number 951, and the presence of the nucleic acid can indicate that the mammal contains a feline immunodeficiency virus. The primer pair can be primer pair number 1001, and the presence of the nucleic acid can indicate that the mammal contains a feline leukemia virus. The primer pair can be primer pair number 151, and the presence of the nucleic acid can indicate that the mammal contains a Borrelia burgdorferi organism.

In another aspect, this document features a method for making an article of manufacture for determining whether or not a mammal contains a pathogen. The method comprising, or consists essentially of: (a) providing a substrate defining a microfluidic chamber, and (b) placing a mixture into the chamber to form the article of manufacture, wherein the mixture comprises at least one primer pair selected from the group consisting of primer pair numbers 951, 1001, 151, 701, 1051, 351, 801, 101, and 1101; wherein the mixture is within the chamber; wherein primer pair number 951 is capable of amplifying, within the chamber, a sequence present in a feline immunodeficiency virus; wherein primer pair number 1001 is capable of amplifying, within the chamber, a sequence present in a feline leukemia virus; wherein primer pair number 151 is capable of amplifying, within the chamber, a sequence present in a Borrelia burgdorferi organism; wherein primer pair number 701 is capable of amplifying, within the chamber, a sequence present in a Dirofilaria immitis organism; wherein primer pair number 1051 is capable of amplifying, within the chamber, a sequence present in a feline parvovirus; wherein primer pair number 351 is capable of amplifying, within the chamber, a sequence present in a canine distemper virus; wherein primer pair number 801 is capable of amplifying, within the chamber, a sequence present in a Ehrlichia canis organism; wherein primer pair number 101 is capable of amplifying, within the chamber, a sequence present in a Bordetella organism; and wherein primer pair number 1101 is capable of amplifying, within the chamber, a sequence present in a Giardia organism. The mixture can be a solid. The mixture can be a liquid. The mammal can be a cat. The mammal can be a dog. The primer pair can be primer pair number 951, and the presence of the nucleic acid can indicate that the mammal contains a feline immunodeficiency virus. The primer pair can be primer pair number 1001, and the presence of the nucleic acid can indicate that the mammal contains a feline leukemia virus. The primer pair can be primer pair number 151, and the presence of the nucleic acid can indicate that the mammal contains a Borrelia burgdorferi organism.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

This document relates to methods and materials involved in detecting pathogens in animals such as companion animals. For example, this document provides nucleic acid primer pairs that can be used in an amplification reaction to detect the presence or absence of a pathogen's nucleic acid within a sample obtained from the animal being tested. This document also provides combinations of nucleic acid primer pairs, nucleic acid arrays (e.g., diagnostic cards) containing nucleic acid primer pairs or combinations of nucleic acid primer pairs, methods for making such nucleic acid arrays, and methods for diagnosing animals infected with a pathogen. The term “pathogen” as used herein includes, without limitation, bacteria, viruses, algae, fungi, parasites, and protozoa.

Nucleic acid primer pairs provided herein are set forth in Table 1. Each primer pair can be used to amplify nucleic acid present in the indicated pathogen. For example, primer pair number 1 can be used to amplify nucleic acid present in an Ancylostoma spp. organism. Primer pair number 1000 can be used to amplify nucleic acid present in a feline immunodeficiency virus.

The nucleic acid primer pairs provided herein can be used separately or in combinations. Such combinations can contain 2, 3, 4, 5, 10, 15, 20, 25, 30, 35, 40, 45, 50, 55, 60, 70, 80, 90, 100, or more different nucleic acid primer pairs from Table 1. When making a combination, any two or more of the provided nucleic acid primer pairs can be arranged into any combination. For example, the first nucleic acid primer pair listed in Table 1 for each of the 32 pathogens can be used to make a collection of 32 different nucleic acid primer pairs. Other combinations include, without limitation, a combination of 32 different nucleic acid primer pairs containing the second nucleic acid primer pair listed in Table 1 for each of the 32 pathogens; a combination of 32 different nucleic acid primer pair containing the third nucleic acid primer pair listed in Table 1 for each of the 32 pathogens; a combination of 32 different nucleic acid primer pairs containing the fourth nucleic acid primer pair listed in Table 1 for each of the 32 pathogens; a combination of 16 different nucleic acid primer pairs containing the first nucleic acid primer pair listed in Table 1 for the first 16 different listed pathogens; a combination of 64 different nucleic acid primer pairs containing the first two nucleic acid primer pairs listed in Table I for each of the 32 pathogens.

In some cases, the combination can contain the first nucleic acid primer pair listed in Table 1 for the following pathogens: Feline immunodeficiency virus, Feline leukemia virus, Borrelia burgdorferi, Dirofilaria immitis, Feline parvovirus, Canine distemper virus, Ehrlichia canis, Bordetella, and Giardia. Such a combination of nucleic acid primer pairs can be used to make a diagnostic card capable of diagnosing infections found in dogs and cats. In some cases, the combination can contain the first nucleic acid primer pair listed in Table 1 for the following pathogens: Feline immunodeficiency virus, Feline leukemia virus, Borrelia burgdorferi, Dirofilaria immitis, Feline parvovirus, Bordetella bronchiseptica, Feline coronavirus, Taenia spp., and Toxoplasma gondii. Such a combination of nucleic acid primer pairs can be used to make a diagnostic card capable of diagnosing infections found in cats. In some cases, the combination can contain the first nucleic acid primer pair listed in Table 1 for the following pathogens: Borrelia burgdorferi, Dirofilaria immitis, Canine distemper virus, Canine parvovirus, Ehrlichia canis, Bordetella spp., Giardia spp., Taenia spp., Mesocestoides corti, and Strongyloides stercoralis. Such a combination of nucleic acid primer pairs can be used to make a diagnostic card capable of diagnosing infections found in dogs.

Each nucleic acid primer pair of a combination can be isolated from the other nucleic acid primer pairs of the combination. For example, each nucleic acid primer pair of a combination can be housed within a separate well of a plastic microtiter plate or a separate chamber of a microfluidic card. In some cases, each nucleic acid primer pair of a combination or a subset of nucleic acid primer pairs of a combination can be housed together. For example, five nucleic acid primer pairs of a combination of 50 nucleic acid primer pairs can be housed within a single well of a plastic microtiter plate with the remaining 45 nucleic acid primer pairs being housed within separate wells.

Any method can be used to make each nucleic acid primer of a nucleic acid primer pair. For example, chemical synthesis techniques such as those described elsewhere (Beaucage and Caruthers, Tetrahedron Lett., 22:1859-62 (1981)) can be used. In addition, nucleic acid primers can be ordered from commercial vendors such as MWG Biotech, Invitrogen, and Operon.

This description also provides arrays having at least one of the nucleic acid primer pairs provided herein. Such arrays can be any type of array including, without limitation, two-dimensional arrays, arrays in microtiter plates (e.g., plates with 48, 96, 384, or 1536 wells), arrays fabricated as an arrangement of microfluidic channels and chambers (e.g., a microfluidic card). In some cases, the array can be microfluidic cards with 8 loading ports each connected through microcapillaries to 48 reaction chambers. In some cases, an array provided herein can contain at least 10 different nucleic acid primer pairs set forth in Table 1 (e.g., at least 20, at least 30, at least 50, at least 100, or at least 200 different nucleic acid primer pairs set forth in Table 1).

In addition to containing any one or more of the nucleic acid primer pairs set forth in Table 1 in any combination, an array can contain nucleic acid primer pairs not listed in Table 1. For example, an array can contain a nucleic acid primer pair designed to amplify host nucleic acid (e.g., dog or cat genomic nucleic acid or mRNA). In some cases, at least 25% (e.g., at least 30%, at least 40%, at least 50%, at least 60%, at least 75%, at least 80%, at least 90%, at least 95%, or 100%) of the nucleic acid primer pairs of an array can be listed in Table 1.

The substrate of an array provided herein can be made of any suitable material (e.g., plastic, glass, silicone, or a metal). In addition, any method can be use to make an array. For example, spotted gelatine or photolithographic techniques can be used to make arrays. In some cases, an array provided herein can be made as follows. A 384-well master plate containing 125 μL of one or more primer pairs in dioinized water at a working concentration of 100 nmole/1 μL of each primer can be constructed. The master plate can be used as a template source, and 1 μL of each master plate well can be transferred to corresponding wells on a 384-well microfluidic card. Spotted reagents can be allowed to dry at room temperature before the final plastic laminate layer of the microfluidic card is attached.

As described herein, the nucleic acid primer pairs set forth in Table 1 can be used to determine whether or not a mammal (e.g., a dog or cat) contains a particular pathogen or set of pathogens. For example, a sample can be obtained from a cat and used in an amplification reaction to determine whether or not a pathogen's nucleic acid is present in the sample. The presence of an amplification product following an amplification reaction using an animal's blood sample and a nucleic acid primer pair designed for a particular pathogen can indicate that that sample contains the pathogen. In such a case, the animal can be diagnosed as being infected with that pathogen. Any type of sample can be used including, without limitation, a biopsy (e.g., punch biopsy, aspiration biopsy, excision biopsy, needle biopsy, or shave biopsy), a tissue section, lymph fluid, blood, serum, saliva, anal swabs, and synovial fluid samples.

Some sample types can be pre-processed to enhance sample quality, such as the concentration of white blood cells through differential centrifugation. Samples can be processed to concentrate the nucleic acid and render it in a form to facilitate successful PCR reactions. This includes, but is not limited to, common methods to disrupt bilipid membranes, such as the use of detergents, digestion of protein complexes, such as the use of proteinase K, and reduction of polymerase inhibitors through the use of selective affinity columns. Commercial kits for purification of DNA, RNA, or total nucleic acid are readily available from, for example, Qiagen and Roche.

Any type of amplification reaction can be used in conjunction with the nucleic acid primer pairs set forth in Table 1 to detect a pathogen. For example, common PCR reactions designed to amplify nucleic acid from DNA or RNA can be used. Detection of RNA viruses or RNA sequences of eukaryote targets, such as ribosomal RNA sequences, can be accomplished by synthesizing cDNA from RNA sequence templates. cDNA synthesis can be accomplished using standard methods using, for example, RNA-dependant DNA polymerases, such as reverse transcriptase. Such reactions can be primed with random oligonucleotide sequences, such as random hexamers and octamers, or by sequence specific oligonucleotide primers, including the same primers used for the PCR reaction. The cDNA synthesis can be performed in a separate reaction vessel from the subsequent PCR reaction (commonly referred to as two-step rtPCR) or in the same reaction vessel as the PCR reaction (commonly referred to as single-step rtPCR).

Purified DNA and cDNA samples can be pooled and added to a PCR master mix containing water, salt buffers, magnesium ions, nucleotide monomers (dATP, dCTP, dGTP and dTTP), native or engineered Thermus aquaticus DNA-dependant DNA polymerase, and an intercalating dye, such as Sybr Green or LC Green. The master mix and sample can then be added to a single loading port of a microfluidic card and dispersed to all the reaction wells using centrifugation. The cards can then be scored to isolate and seal each reaction chamber prior to thermocycling. The cards can be individually thermocycled using commodity block thermocyclers or many cards thermocycled simultaneously using air- or water-based thermocyclers such as the BioOven or the H20BIT, respectively.

Positive PCR amplification reactions can be detected during thermocycling for quantitative or qualitative analysis (real time PCR) or after completion of thermocycling (qualitative end-point PCR). Signals can be detected through fluorescence-channel emission of substrate bound intercalating dyes using commodity real-time PCR capable PCR platforms or by end-point reads using microplate scanner platforms. Both types of platforms can be used for melting-point analysis for validation of positive signals.

TABLE 1
Primer pairs that can be used to detect the indicated pathogen.

| | | | | | |
|---|---|---|---|---|---|
| | | | SEQ | | SEQ |
| Primer | | | ID | | ID |
| Pair No. | Pathogen/Parasite | Forward Primer Sequence | NO: | Reverse Primer Sequence | NO: |

| | | | | | |
|---|---|---|---|---|---|
| 1 | Ancylostoma spp | ATTTTGATTTTACCAGCTTTTGGTAT | 1 | AAATACTTTTACACCAGTGGGAACA | 2 |
| 2 | Ancylostoma spp | GATTTGGATTCTCGTGCTTATTTTA | 3 | TGAATTTGACAATACAACACCAGTTA | 4 |
| 3 | Ancylostoma spp | TTTTGATTTTACCAGCTTTTGGTAT | 5 | CTTTTACACCAGTAGGAACAGCAAT | 6 |
| 4 | Ancylostoma spp | TTTTGATTTTACCAGCTTTTGGTAT | 5 | AAATACTTTTACACCAGTGGGAACA | 2 |
| 5 | Ancylostoma spp | TTTTGATTTTACCAGCTTTTGGTAT | 5 | CACAGTATACATATGATGAGCCCAC | 7 |
| 6 | Ancylostoma spp | TTTTGATTTTACCAGCTTTTGGTAT | 5 | CACAGTATACATATGATGGGCTCAC | 8 |
| 7 | Ancylostoma spp | TTTTGATTTTACCAGCTTTTGGTAT | 5 | CAGTATACATATGATGGGCTCACAC | 9 |
| 8 | Ancylostoma spp | TTTTGATTTTACCAGCTTTTGGTAT | 5 | CAGTATACATATGATGAGCCCACAC | 10 |
| 9 | Ancylostoma spp | TTTTGATTTTACCAGCTTTTGGTAT | 5 | ACCCACAGTATACATATGATGAGCC | 11 |
| 10 | Ancylostoma spp | TTTTGATTTTACCAGCTTTTGGTAT | 5 | AGTAGGAACAGCAATCACCATAGTT | 12 |
| 11 | Ancylostoma spp | TTTTGATTTTACCAGCTTTTGGTAT | 5 | ACTTTTACACCAGTAGGAACAGCAA | 13 |
| 12 | Ancylostoma spp | TTTTGATTTTACCAGCTTTTGGTAT | 5 | AAAACCCACAATAATAAAGGTTGAA | 14 |
| 13 | Ancylostoma spp | TTATACATGTCTGAACGAGAAAGCA | 15 | TGGAGTTGGAAAGCTTTATTCTCTC | 16 |
| 14 | Ancylostoma spp | GTATGCAAAGACGGATTCTACAGAG | 17 | TGGAGTTGGAAAGCTTTATTCTCTC | 16 |
| 15 | Ancylostoma spp | TTTGATTTTACCAGCTTTTGGTATT | 18 | CTTTTACACCAGTAGGAACAGCAAT | 6 |
| 16 | Ancylostoma spp | TTTGATTTTACCAGCTTTTGGTATT | 18 | AAATACTTTTACACCAGTGGGAACA | 2 |
| 17 | Ancylostoma spp | TTTGATTTTACCAGCTTTTGGTATT | 18 | CACAGTATACATATGATGAGCCCAC | 7 |
| 18 | Ancylostoma spp | TTTGATTTTACCAGCTTTTGGTATT | 18 | CACAGTATACATATGATGGGCTCAC | 8 |
| 19 | Ancylostoma spp | TTTGATTTTACCAGCTTTTGGTATT | 18 | ACCCACAGTATACATATGATGAGCC | 11 |
| 20 | Ancylostoma spp | TTATACATGTCTGAACGAGAAAGCA | 15 | TTGGAGTTGGAAAGCTTTATTCTCT | 19 |
| 21 | Ancylostoma spp | TATACATGTCTGAACGAGAAAGCAA | 20 | TGGAGTTGGAAAGCTTTATTCTCT | 19 |
| 22 | Ancylostoma spp | TTATACATGTCTGAACGAGAAAGCA | 15 | TGGAGTTGGAAAGCTTTATTCTCT | 21 |
| 23 | Ancylostoma spp | TATACATGTCTGAACGAGAAAGCAA | 20 | TGGAGTTGGAAAGCTTTATTCTCT | 21 |
| 24 | Ancylostoma spp | ATACATGTCTGAACGAGAAAGCAAC | 22 | TTGGAGTTGGAAAGCTTTATTCTCT | 19 |
| 25 | Ancylostoma spp | GAATGCGACCAACATGAGATTATAC | 23 | TTGGAGTTGGAAAGCTTTATTCTCT | 19 |
| 26 | Ancylostoma spp | TATACATGTCTGAACGAGAAAGCAA | 20 | TTTTGGAGTTGGAAAGCTTTATTCT | 24 |
| 27 | Ancylostoma spp | TTATACATGTCTGAACGAGAAAGCA | 15 | TTTTGGAGTTGGAAAGCTTTATTCT | 24 |
| 28 | Ancylostoma spp | TATACATGTCTGAACGAGAAAGCAA | 20 | TTTGGAGTTGGAAAGCTTTATTCT | 25 |
| 29 | Ancylostoma spp | TTATACATGTCTGAACGAGAAAGCA | 15 | TTTGGAGTTGGAAAGCTTTATTCT | 25 |
| 30 | Ancylostoma spp | ATACATGTCTGAACGAGAAAGCAAC | 22 | TTTTGGAGTTGGAAAGCTTTATTCT | 24 |
| 31 | Ancylostoma spp | GAATGCGACCAACATGAGATTATAC | 23 | TTTTGGAGTTGGAAAGCTTTATTCT | 24 |
| 32 | Ancylostoma spp | GTTTATTACCTCCTGCTTGCGTAT | 26 | TTGGAGTTGGAAAGCTTTATTCTCT | 19 |
| 33 | Ancylostoma spp | GTGGTTGTTTATTACCTCCTGCTT | 27 | TTGGAGTTGGAAAGCTTTATTCTCT | 19 |
| 34 | Ancylostoma spp | GTATGCAAAGACGGATTCTACAGAG | 17 | TTTTGGAGTTGGAAAGCTTTATTCT | 24 |
| 35 | Ancylostoma spp | GTATGCAAAGACGGATTCTACAGAG | 17 | TTTGGAGTTGGAAAGCTTTATTCT | 25 |
| 36 | Ancylostoma spp | AGTGTAGCTTGTGGCACTGTTTG | 28 | TAACAGAAACATCGTTGTTATACTAGCC | 29 |
| 37 | Ancylostoma spp | CAGTGTAGCTTGTGGCACTGTTT | 30 | TAACAGAAACATCGTTGTTATACTAGCC | 29 |
| 38 | Ancylostoma spp | GTGTAGCTTGTGGCACTGTTTGT | 31 | TAACAGAAACATCGTTGTTATACTAGCC | 29 |
| 39 | Ancylostoma spp | TTGATTTTACCAGCTTTTGGTATTG | 32 | AAATACTTTTACACCAGTGGGAACA | 2 |
| 40 | Ancylostoma spp | TTGATTTTACCAGCTTTTGGTATTG | 32 | TAAAATAAGCACGAGAATCCAAATC | 33 |
| 41 | Ancylostoma spp | TTGATTTTACCAGCTTTTGGTATTG | 32 | CACAGTATACATATGATGGGCTCAC | 8 |
| 42 | Ancylostoma spp | TTGATTTTACCAGCTTTTGGTATTG | 32 | CACAGTATACATATGATGAGCCCAC | 7 |
| 43 | Ancylostoma spp | TTGATTTTACCAGCTTTTGGTATTG | 32 | ACCCACAGTATACATATGATGAGCC | 11 |
| 44 | Ancylostoma spp | TTGATTTTACCAGCTTTTGGTATTG | 32 | ACTTTTACACCAGTAGGAACAGCAA | 13 |
| 45 | Ancylostoma spp | TTGATTTTACCAGCTTTTGGTATTG | 32 | TTTTACACCAGTAGGAACAGCAATC | 34 |
| 46 | Ancylostoma spp | CAGTGTAGCTTGTGGCACTGTTT | 30 | ATAACAGAAACATCGTTGTCATACTAGC | 35 |
| 47 | Ancylostoma spp | AGTGTAGCTTGTGGCACTGTTTG | 28 | ATAACAGAAACATCGTTGTCATACTAGC | 35 |
| 48 | Ancylostoma spp | CAGTGTAGCTTGTGGCACTGTTT | 30 | CAAATAACAGAAACATCGTTGTCATACT | 36 |
| 49 | Ancylostoma spp | AGTGTAGCTTGTGGCACTGTTTG | 28 | CAAATAACAGAAACATCGTTGTCATACT | 36 |
| 50 | Ancylostoma spp | CAGTGTAGCTTGTGGCACTGTTT | 30 | AAATAACAGAAACATCGTTGTCATACTA | 37 |
| 51 | Bartonella spp | ACAGATCGAAGAAACAACTTCTGAC | 38 | GACGAACAATATTGATACCAGCTTC | 39 |
| 52 | Bartonella spp | AGGCTGGTATTGAAGAATTACAAAA | 40 | ATAACAGAACGAACATCAGCAAAAT | 41 |
| 53 | Bartonella spp | TAAAAATGATCCTTTCCGTCTTATG | 42 | ACATTTCAGTTGGAAAACCTAAAGC | 43 |
| 54 | Bartonella spp | AATGAAGCATGCCTAAAAATGTTAC | 44 | CAGAATAGAAATCAACATTCGGATAA | 45 |
| 55 | Bartonella spp | AATGAAGCATGCCTAAAAATGTTAC | 44 | AATAGAAATCAACATTCGGATAAAG | 46 |
| 56 | Bartonella spp | ATAAAAATGATCCTTTCCGTCTTATG | 47 | CCTAAAGCTTTTAGTGTAATGCCAG | 48 |
| 57 | Bartonella spp | AGATAAAAATGATCCTTTCCGTCTTA | 49 | CCTAAAGCTTTTAGTGTAATGCCAG | 48 |
| 58 | Bartonella spp | TAAAAATGATCCTTTCCGTCTTATG | 42 | CTAAAGCTTTTAGTGTAATGCCAGAA | 50 |
| 59 | Bartonella spp | GATAAAAATGATCCTTTCCGTCTTAT | 51 | CCTAAAGCTTTTAGTGTAATGCCAG | 48 |
| 60 | Bartonella spp | ATAAAAATGATCCTTTCCGTCTTATG | 47 | AAAGCTTTTAGTGTAATGCCAGAAT | 52 |
| 61 | Bartonella spp | ATAAAAATGATCCTTTCCGTCTTATG | 47 | AGCCTAAAGCTTTTAGTGTAATGCC | 53 |
| 62 | Bartonella spp | AGATAAAAATGATCCTTTCCGTCTTA | 49 | AGCCTAAAGCTTTTAGTGTAATGCC | 53 |
| 63 | Bartonella spp | ATAAAAATGATCCTTTCCGTCTTATG | 47 | TGTAATGCCAGAATAGAAATCAACA | 54 |
| 64 | Bartonella spp | TAAAAATGATCCTTTCCGTCTTATG | 42 | AACATTTCAGTTGGAAAACCTAAAG | 55 |
| 65 | Bartonella spp | AGATAAAAATGATCCTTTCCGTCTTA | 49 | TGTAATGCCAGAATAGAAATCAACA | 54 |
| 66 | Bartonella spp | TAAAAATGATCCTTTCCGTCTTATG | 42 | AAACATTTCAGTTGGAAAACCTAAAG | 56 |
| 67 | Bartonella spp | AGATAAAAATGATCCTTTCCGTCTTA | 49 | AATGCCAGAATAGAAATCAACATTC | 57 |
| 68 | Bartonella spp | TAAAAATGATCCTTTCCGTCTTATG | 42 | CAGAATAGAAATCAACATTCGGATAA | 45 |
| 69 | Bartonella spp | ATAAAAATGATCCTTTCCGTCTTATG | 47 | TGTAATGCCAGAATAAAAATCAACAT | 58 |
| 70 | Bartonella spp | AATGAAGCATGCCTAAAAATGTTAC | 44 | CAGAATAGAAATCAACATTCGGATA | 59 |
| 71 | Bartonella spp | AATGAAGCATGCCTAAAAATGTTAC | 44 | TTTTAAGACTTCATGACAGGTTTGTT | 60 |
| 72 | Bartonella spp | AAAATGTTACAAGAAATAGGTTCCGT | 61 | TGTAATGCCAGAATAGAAATCAACA | 54 |
| 73 | Bartonella spp | ATGTTACAAGAAATAGGTTCCGTCA | 62 | TGTAATGCCAGAATAGAAATCAACAT | 63 |
| 74 | Bartonella spp | ATGTTACAAGAAATAGGTTCCGTCA | 62 | TAATGCCAGAATAGAAATCAACATTC | 64 |
| 75 | Bartonella spp | AAAATGTTACAAGAAATAGGTTCCGT | 61 | AATGCCAGAATAGAAATCAACATTC | 57 |
| 76 | Bartonella spp | AGCATGCCTAAAAATGTTACAAGAA | 65 | TAATGCCAGAATAGAAATCAACATTC | 64 |
| 77 | Bartonella spp | AAGCATGCCTAAAAATGTTACAAGA | 66 | TAATGCCAGAATAGAAATCAACATTC | 64 |
| 78 | Bartonella spp | TACAAGAAATAGGTTCCGTCAAAAG | 67 | TGTAATGCCAGAATAGAAATCAACAT | 63 |
| 79 | Bartonella spp | TACAAGAAATAGGTTCCGTCAAAAG | 67 | TAATGCCAGAATAGAAATCAACATTC | 64 |
| 80 | Bartonella spp | ATAAAAATGATCCTTTCCGTCTTATG | 47 | TTCAGGGCAATATTTTCAAGTTCTA | 68 |
| 81 | Bartonella spp | ATAAAAATGATCCTTTCCGTCTTATG | 47 | TCAGGGCAATATTTTCAAGTTCTAT | 69 |
| 82 | Bartonella spp | AGATAAAAATGATCCTTTCCGTCTTA | 49 | TTCAGGGCAATATTTTCAAGTTCTA | 68 |
| 83 | Bartonella spp | ATAAAAATGATCCTTTCCGTCTTATG | 47 | ATTCAGGGCAATATTTTCAAGTTCT | 70 |
| 84 | Bartonella spp | AGATAAAAATGATCCTTTCCGTCTTA | 49 | TCAGGGCAATATTTTCAAGTTCTAT | 69 |
| 85 | Bartonella spp | AGATAAAAATGATCCTTTCCGTCTTA | 49 | ATTCAGGGCAATATTTTCAAGTTCT | 70 |
| 86 | Bartonella spp | TAAAAATGATCCTTTCCGTCTTATG | 42 | TTCAGGGCAATATTTTCAAGTTCTAT | 71 |
| 87 | Bartonella spp | TAAAAATGATCCTTTCCGTCTTATG | 42 | ATTCAGGGCAATATTTTCAAGTTCTA | 72 |
| 88 | Bartonella spp | AATGAAGCATGCCTAAAAATGTTAC | 44 | TTCAGGGCAATATTTTCAAGTTCTA | 68 |
| 89 | Bartonella spp | AATGAAGCATGCCTAAAAATGTTAC | 44 | TCAGGGCAATATTTTCAAGTTCTAT | 69 |
| 90 | Bartonella spp | AATGAAGCATGCCTAAAAATGTTAC | 44 | ATAGAAATCAACATTCGGATAAAGC | 73 |
| 91 | Bartonella spp | AAATGTTACAAGAAATAGGTTCCGTC | 74 | TGTAATGCCAGAATAGAAATCAACA | 54 |
| 92 | Bartonella spp | AAAAATGTTACAAGAAATAGGTTCCG | 75 | TGTAATGCCAGAATAGAAATCAACA | 54 |
| 93 | Bartonella spp | AAAAATGTTACAAGAAATAGGTTCCG | 75 | AATGCCAGAATAGAAATCAACATTC | 57 |
| 94 | Bartonella spp | AAATGTTACAAGAAATAGGTTCCGTC | 74 | AATGCCAGAATAGAAATCAACATTC | 57 |
| 95 | Bartonella spp | GTTACAAGAAATAGGTTCCGTCAAA | 76 | TGTAATGCCAGAATAGAAATCAACAT | 63 |
| 96 | Bartonella spp | AAGAAATAGGTTCCGTCAAAAGAAT | 77 | TGTAATGCCAGAATAGAAATCAACAT | 63 |
| 97 | Bartonella spp | TAAAAATGATCCTTTCCGTCTTATG | 42 | AATAGAAATCAACATTCGGATAAAGC | 46 |
| 98 | Bartonella spp | GTTACAAGAAATAGGTTCCGTCAAA | 76 | TAATGCCAGAATAGAAATCAACATTC | 64 |
| 99 | Bartonella spp | TTACAAGAAATAGGTTCCGTCAAAA | 78 | TGTAATGCCAGAATAGAAATCAACAT | 63 |
| 100 | Bartonella spp | TTACAAGAAATAGGTTCCGTCAAAA | 78 | TAATGCCAGAATAGAAATCAACATTC | 64 |
| 101 | Bordetella spp | CTGAACGAAATCAACAACAACCT | 79 | GTCTTGGACTTCAGGACCTTGAT | 80 |
| 102 | Bordetella spp | GTATTCGGGGCCAATGAGCTTACAG | 81 | TCCGATACATCCACGCCATAC | 82 |
| 103 | Bordetella spp | GTAGGGCTTCACACGTCATACAAT | 83 | TTAGGCTAACTACTTCTGGTAGAACCC | 84 |
| 104 | Bordetella spp | GTAGGGCTTCACACGTCATACAAT | 83 | GCTAACTACTTCTGGTAGAACCCACT | 85 |
| 105 | Bordetella spp | GTAGGGCTTCACACGTCATACAAT | 83 | TAGGCTAACTACTTCTGGTAGAACCC | 86 |
| 106 | Bordetella spp | GTAGGGCTTCACACGTCATACAAT | 83 | TAACTACTTCTGGTAGAACCCACTCC | 87 |
| 107 | Bordetella spp | GTAGGGCTTCACACGTCATACAAT | 83 | AACTACTTCTGGTAGAACCCACTCC | 88 |
| 108 | Bordetella spp | GTAGGGCTTCACACGTCATACAAT | 83 | AGGCTAACTACTTCTGGTAGAACCC | 89 |
| 109 | Bordetella spp | GTAGGGCTTCACACGTCATACAAT | 83 | GCTAACTACTTCTGGTAGAACCCAC | 90 |
| 110 | Bordetella spp | GTAGGGCTTCACACGTCATACAAT | 83 | GGCTAACTACTTCTGGTAGAACCCA | 91 |
| 111 | Bordetella spp | TAGGGCTTCACACGTCATACAAT | 92 | GCTAACTACTTCTGGTAGAACCCACT | 85 |
| 112 | Bordetella spp | TAGGGCTTCACACGTCATACAAT | 92 | TAGGCTAACTACTTCTGGTAGAACCC | 86 |
| 113 | Bordetella spp | TAGGGCTTCACACGTCATACAAT | 92 | TAACTACTTCTGGTAGAACCCACTCC | 87 |
| 114 | Bordetella spp | GTAGGGCTTCACACGTCATACAA | 93 | GCTAACTACTTCTGGTAGAACCCACT | 85 |
| 115 | Bordetella spp | GTAGGGCTTCACACGTCATACAA | 93 | TAGGCTAACTACTTCTGGTAGAACCC | 86 |
| 116 | Bordetella spp | GTAGGGCTTCACACGTCATACAA | 93 | TAACTACTTCTGGTAGAACCCACTCC | 87 |
| 117 | Bordetella spp | GCTTCACACGTCATACAATGGTC | 94 | GCTAACTACTTCTGGTAGAACCCACT | 85 |
| 118 | Bordetella spp | GCTTCACACGTCATACAATGGTC | 94 | TAGGCTAACTACTTCTGGTAGAACCC | 86 |
| 119 | Bordetella spp | GCTTCACACGTCATACAATGGTC | 94 | TAACTACTTCTGGTAGAACCCACTCC | 87 |
| 120 | Bordetella spp | ACTCTAATGAGACTGCCGGTGAC | 95 | GCTAACTACTTCTGGTAGAACCCACT | 85 |
| 121 | Bordetella spp | GAAAAACCTTACCTACCCTTGACAT | 96 | GTCTCATTAGAGTGCCCTTTCGTAG | 97 |
| 122 | Bordetella spp | GAAAAACCTTACCTACCCTTGACAT | 96 | AGTCTCATTAGAGTGCCCTTTCGTA | 98 |
| 123 | Bordetella spp | GAAAAACCTTACCTACCCTTGACAC | 99 | GTCTCATTAGAGTGCCCTTTCGTAG | 97 |
| 124 | Bordetella spp | GAAAAACCTTACCTACCCTTGACAC | 99 | AGTCTCATTAGAGTGCCCTTTCGTA | 98 |
| 125 | Bordetella spp | GAAAAACCTTACCTACCCTTGACA | 100 | AGTCTCATTAGAGTGCCCTTTCGTA | 98 |
| 126 | Bordetella spp | GAAAAACCTTACCTACCCTTGACA | 100 | GTCTCATTAGAGTGCCCTTTCGTAG | 97 |
| 127 | Bordetella spp | CGAAAAACCTTACCTACCCTTGAC | 101 | GTCTCATTAGAGTGCCCTTTCGTAG | 97 |
| 128 | Bordetella spp | CGAAAAACCTTACCTACCCTTGAC | 101 | AGTCTCATTAGAGTGCCCTTTCGTA | 98 |
| 129 | Bordetella spp | GTAGGGCTTCACACGTCATACAAT | 83 | GGCTAACTACTTCTGGTAGAACCC | 102 |
| 130 | Bordetella spp | TAGGGCTTCACACGTCATACAAT | 92 | AACTACTTCTGGTAGAACCCACTCC | 88 |
| 131 | Bordetella spp | TAGGGCTTCACACGTCATACAAT | 92 | AGGCTAACTACTTCTGGTAGAACCC | 89 |
| 132 | Bordetella spp | GTAGGGCTTCACACGTCATACAA | 93 | AACTACTTCTGGTAGAACCCACTCC | 88 |
| 133 | Bordetella spp | GTAGGGCTTCACACGTCATACAA | 93 | AGGCTAACTACTTCTGGTAGAACCC | 89 |
| 134 | Bordetella spp | TAGGGCTTCACACGTCATACAAT | 92 | GCTAACTACTTCTGGTAGAACCCAC | 90 |
| 135 | Bordetella spp | TAGGGCTTCACACGTCATACAAT | 92 | GGCTAACTACTTCTGGTAGAACCCA | 91 |
| 136 | Bordetella spp | GCTTCACACGTCATACAATGGTC | 94 | AACTACTTCTGGTAGAACCCACTCC | 88 |
| 137 | Bordetella spp | GCTTCACACGTCATACAATGGTC | 94 | AGGCTAACTACTTCTGGTAGAACCC | 89 |
| 138 | Bordetella spp | GTAGGGCTTCACACGTCATACAAT | 83 | ACTACTTCTGGTAGAACCCACTCC | 103 |
| 139 | Bordetella spp | GTAGGGCTTCACACGTCATACAA | 93 | GCTAACTACTTCTGGTAGAACCCAC | 90 |
| 140 | Bordetella spp | GTAGGGCTTCACACGTCATACAA | 93 | GGCTAACTACTTCTGGTAGAACCCA | 91 |
| 141 | Bordetella spp | GCTTCACACGTCATACAATGGTC | 94 | GCTAACTACTTCTGGTAGAACCCAC | 90 |
| 142 | Bordetella spp | GCTTCACACGTCATACAATGGTC | 94 | GGCTAACTACTTCTGGTAGAACCCA | 91 |
| 143 | Bordetella spp | GTAGGGCTTCACACGTCATACAAT | 83 | CTACTTCTGGTAGAACCCACTCCC | 104 |
| 144 | Bordetella spp | ACTCTAATGAGACTGCCGGTGAC | 95 | AACTACTTCTGGTAGAACCCACTCC | 88 |
| 145 | Bordetella spp | ACTCTAATGAGACTGCCGGTGAC | 95 | AGGCTAACTACTTCTGGTAGAACCC | 89 |
| 146 | Bordetella spp | ACTCTAATGAGACTGCCGGTGAC | 95 | GCTAACTACTTCTGGTAGAACCCAC | 90 |
| 147 | Bordetella spp | ACTCTAATGAGACTGCCGGTGAC | 95 | GGCTAACTACTTCTGGTAGAACCCA | 91 |
| 148 | Bordetella spp | GAAAAACCTTACCTACCCTTGACAT | 96 | ATTGTATGACGTGTGAAGCCCTAC | 105 |
| 149 | Bordetella spp | GAAAAACCTTACCTACCCTTGACAC | 99 | ATTGTATGACGTGTGAAGCCCTAC | 105 |
| 150 | Bordetella spp | TAGTTGCTACGAAAGGGCACTCTA | 106 | GATTACTAGCGATTCCGACTTCAT | 107 |
| 151 | Borrelia burgdorferi | ATAGCATGTAAGCAAAATGTTAGCG | 108 | GTGCTGAGATCCTCAGAAACTGTTA | 109 |
| 152 | Borrelia burgdorferi | GTGAAATGAAAGTTCTTGTAAGCAAA | 110 | GTGCTGAGATCTTCAGAAACTGTTA | 111 |
| 153 | Borrelia burgdorferi | TGAAATGAAAGTTCTTGTAAGCAAAG | 112 | GTGCTGAGATCTTCAGAAACTGTTA | 111 |
| 154 | Borrelia burgdorferi | GTTCTTGTAAGCAAAGAAAAAGACAA | 113 | GTGCTGAGATCTTCAGAAACTGTTA | 111 |
| 155 | Borrelia burgdorferi | GAAATGAAAGTTCTTGTAAGCAAAGA | 114 | GTGCTGAGATCTTCAGAAACTGTTA | 111 |
| 156 | Borrelia burgdorferi | TTCTTGTAAGCAAAGAAAAAGACAAA | 115 | GTGCTGAGATCTTCAGAAACTGTTA | 111 |
| 157 | Borrelia burgdorferi | TCTTGTAAGCAAAGAAAAAGACAAAG | 116 | GTGCTGAGATCTTCAGAAACTGTTA | 111 |
| 158 | Borrelia burgdorferi | GTGAAATGAAAGTTCTTGTAAGCAAA | 110 | GTGCTTAGATCGTCAGAAACTGTTA | 117 |
| 159 | Borrelia burgdorferi | TGAAATGAAAGTTCTTGTAAGCAAAG | 112 | GTGCTTAGATCGTCAGAAACTGTTA | 117 |
| 160 | Borrelia burgdorferi | ATAGCATGTAAGCAAAATGTTAGCAG | 118 | GTTCCTTTAAGCTCAAGCTTGTCTAC | 119 |
| 161 | Borrelia burgdorferi | ATAGCATGTAAGCAAAATGTTAGCAG | 118 | TTCCTTTAAGCTCAAGCTTGTCTACT | 120 |
| 162 | Borrelia burgdorferi | ATAGCATGTAAGCAAAATGTTAGCAG | 118 | TTTAAGCTCAAGCTTGTCTACTGTTG | 121 |
| 163 | Borrelia burgdorferi | GTGAAATGAAAGTTCTTGTAAGCAAA | 110 | GTGCTGAGATCCTCAGAAACTGTTA | 109 |
| 164 | Borrelia burgdorferi | TGAAATGAAAGTTCTTGTAAGCAAAG | 112 | GTGCTGAGATCCTCAGAAACTGTTA | 109 |
| 165 | Borrelia burgdorferi | GTTCTTGTAAGCAAAGAAAAAGACAA | 113 | GTGCTTAGATCGTCAGAAACTGTTA | 117 |
| 166 | Borrelia burgdorferi | GAAATGAAAGTTCTTGTAAGCAAAGA | 114 | GTGCTTAGATCGTCAGAAACTGTTA | 117 |
| 167 | Borrelia burgdorferi | TTCTTGTAAGCAAAGAAAAAGACAAA | 115 | GTGCTTAGATCGTCAGAAACTGTTA | 117 |
| 168 | Borrelia burgdorferi | ATAGCATGTAAGCAAAATGTTAGCAG | 118 | AATTTTACTTTGCTTTTGTCAGCTTT | 122 |
| 169 | Borrelia burgdorferi | TCTTGTAAGCAAAGAAAAAGACAAAG | 116 | GTGCTTAGATCGTCAGAAACTGTTA | 117 |
| 170 | Borrelia burgdorferi | ATAGCATGTAAGCAAAATGTTAGCAG | 118 | CAGATCCATTGTTTTTATCAGATGTT | 123 |
| 171 | Borrelia burgdorferi | TACCTGGTGAAATGAAAGTTCTTGTA | 124 | GTGCTGAGATCCTCAGAAACTGTTA | 109 |
| 172 | Borrelia burgdorferi | TTCTTGTAAGCAAAGAAAAAGACAAA | 115 | GTGCTGAGATCCTCAGAAACTGTTA | 109 |
| 173 | Borrelia burgdorferi | ATAGCATGTAAGCAAAATGTTAGCAG | 118 | ATCCATTGTTTTTATCAGATGTTCCT | 125 |
| 174 | Borrelia burgdorferi | TCTTGTAAGCAAAGAAAAAGACAAAG | 116 | GTGCTGAGATCCTCAGAAACTGTTA | 109 |
| 175 | Borrelia burgdorferi | CTTGTAAGCAAAGAAAAAGACAAAGA | 126 | GTGCTGAGATCTTCAGAAACTGTTA | 111 |
| 176 | Borrelia burgdorferi | TACGATTTCTTATGTTGAGGGAATG | 127 | GCACAAACTTTTAAAGCTCCTCTAAC | 128 |
| 177 | Borrelia burgdorferi | TTGTAAGCAAAGAAAAAGACAAAGAC | 129 | GTGCTGAGATCTTCAGAAACTGTTA | 111 |
| 178 | Borrelia burgdorferi | ATGTAAGCAAAATGTTAGCAGCCTT | 130 | GTGCTGAGATCTTCAGAAACTGTTA | 111 |
| 179 | Borrelia burgdorferi | ATAGCATGTAAGCAAAATGTTAGCAG | 118 | TCCTTTAAGCTCAAGATTGTCTACTG | 131 |
| 180 | Borrelia burgdorferi | TGAAATGAAAGTTCTTGTAAGCAAA | 132 | GTGCTGAGATCTTCAGAAACTGTTA | 111 |
| 181 | Borrelia burgdorferi | TACGATTTCTTATGTTGAGGGAATG | 127 | ATAATCAACAAAGGCTTATTTGTTCC | 133 |
| 182 | Borrelia burgdorferi | TACGATTTCTTATGTTGAGGGAATG | 127 | CTAAAACTTTCTCAAGAACCGGTAAA | 134 |
| 183 | Borrelia burgdorferi | TACGATTTCTTATGTTGAGGGAATG | 127 | CAAAGGCTTATTTGTTCCTAAAACTT | 135 |
| 184 | Borrelia burgdorferi | TGAAAGTTCTTGTAAGCAAAGAAAA | 136 | GTGCTGAGATCTTCAGAAACTGTTA | 111 |
| 185 | Borrelia burgdorferi | TACGATTTCTTATGTTGAGGGAATG | 127 | TCCTTCAATATCTTCAGCAATAATCA | 137 |
| 186 | Borrelia burgdorferi | TACGATTTCTTATGTTGAGGGAATG | 127 | CAAGAACCGGTAAAAGCTCTTTAATA | 138 |
| 187 | Borrelia burgdorferi | TTCTTGTAAGCAAAGAAAAAGACAA | 139 | GTGCTGAGATCTTCAGAAACTGTTA | 111 |
| 188 | Borrelia burgdorferi | ATAGCATGTAAGCAAAATGTTAGCAG | 118 | TACTTTGCTTTTGTCAGCTTTTACG | 140 |
| 189 | Borrelia burgdorferi | CTTGTAAGCAAAGAAAAAGACAAAGA | 126 | GTGCTTAGATCGTCAGAAACTGTTA | 117 |
| 190 | Borrelia burgdorferi | TTGTAAGCAAAGAAAAAGACAAAGAC | 129 | GTGCTTAGATCGTCAGAAACTGTTA | 117 |
| 191 | Borrelia burgdorferi | ATGTAAGCAAAATGTTAGCAGCCTT | 130 | GTGCTTAGATCGTCAGAAACTGTTA | 117 |
| 192 | Borrelia burgdorferi | CTTGTAAGCAAAGAAAAAGACAAAGA | 126 | GTGCTGAGATCCTCAGAAACTGTTA | 109 |
| 193 | Borrelia burgdorferi | TCTTGTAAGCAAAGAAAAAGACAAA | 141 | GTGCTGAGATCTTCAGAAACTGTTA | 111 |
| 194 | Borrelia burgdorferi | ATAGCATGTAAGCAAAATGTTAGCAG | 118 | CCTTTAAGCTCAAGCTTGTCTACTG | 142 |
| 195 | Borrelia burgdorferi | ATAGCATGTAAGCAAAATGTTAGCAG | 118 | TTCCTTTAAGCTCAAGCTTGTCTAC | 143 |
| 196 | Borrelia burgdorferi | GAGAAAAACAGCGTTTCAGTAGATT | 144 | GTGCTTAGATCGTCAGAAACTGTTA | 117 |
| 197 | Borrelia burgdorferi | ATAGCATGTAAGCAAAATGTTAGCAG | 118 | TCCTTTAAGCTCAAGCTTGTCTACT | 145 |
| 198 | Borrelia burgdorferi | ATAGCATGTAAGCAAAATGTTAGCAG | 118 | AATTTTACTTTGCTTTTGTCAGCTT | 146 |
| 199 | Borrelia burgdorferi | GTAAGCAAAGAAAAAGACAAAGACG | 147 | GTGCTTAGATCGTCAGAAACTGTTAAT | 148 |
| 200 | Borrelia burgdorferi | TGAAATGAAAGTTCTTGTAAGCAAA | 132 | GTGCTTAGATCGTCAGAAACTGTTA | 117 |
| 201 | Campylobacter spp | TCAAGATGGACAAAGTTTAAAAACAA | 149 | CTAGATGAAATTCTTCCTCCTGTTTC | 150 |
| 202 | Campylobacter spp | TCAAGATGGACAAAGTTTAAAAACAA | 149 | CACTAGATGAAATTCTTCCTCCTGTT | 151 |
| 203 | Campylobacter spp | TCAAGATGGACAAAGTTTAAAAACAA | 149 | TTTCAAAGCGTGTTAAACCTATCTTA | 152 |
| 204 | Campylobacter spp | TCAAGATGGACAAAGTTTAAAAACAA | 149 | CAAAGCGTGTTAAACCTATCTTAGAA | 153 |
| 205 | Campylobacter spp | TCAAGATGGACAAAGTTTAAAAACAA | 149 | TTCAAAGCGTGTTAAACCTATCTTAG | 154 |
| 206 | Campylobacter spp | TCAAGATGGACAAAGTTTAAAAACAA | 149 | CCTATCTTAGAAGATTGAGTTGCTCC | 155 |
| 207 | Campylobacter spp | TCAAGATGGACAAAGTTTAAAAACAA | 149 | TAGATGAAATTCTTCCTCCTGTTTC | 156 |
| 208 | Campylobacter spp | TCAAGATGGACAAAGTTTAAAAACAA | 149 | AGATGAAATTCTTCCTCCTGTTTCA | 157 |
| 209 | Campylobacter spp | TCAAGATGGACAAAGTTTAAAAACAA | 149 | GTTTCAAAGCGTGTTAAACCTATCT | 158 |
| 210 | Campylobacter spp | TCAAGATGGACAAAGTTTAAAAACAA | 149 | TTCAAAGCGTGTTAAACCTATCTTA | 159 |
| 211 | Campylobacter spp | TCAAGATGGACAAAGTTTAAAAACAA | 149 | CACCACTAGATGAAATTCTTCCTCC | 160 |
| 212 | Campylobacter spp | TCAAGATGGACAAAGTTTAAAAACAA | 149 | ACCACTAGATGAAATTCTTCCTCCT | 161 |
| 213 | Campylobacter spp | TCAAGATGGACAAAGTTTAAAAACAA | 149 | TGTTTCAAAGCGTGTTAAACCTATC | 162 |
| 214 | Campylobacter spp | TCAAGATGGACAAAGTTTAAAAACAA | 149 | TAAATTGCACTTCACCACTAGATGA | 163 |
| 215 | Campylobacter spp | TCAAGATGGACAAAGTTTAAAAACAA | 149 | GCACTTCACCACTAGATGAAATTCT | 164 |
| 216 | Campylobacter spp | TCAAGATGGACAAAGTTTAAAAACAA | 149 | ACCGATTTGAAATTCTTGATTGATA | 165 |
| 217 | Campylobacter spp | TCAAGATGGACAAAGTTTAAAAACAA | 149 | AATTGCACTTCACCACTAGATGAAA | 166 |
| 218 | Campylobacter spp | TCAAGATGGACAAAGTTTAAAAACAA | 149 | GAACTAGCACCGATTTGAAATTCTT | 167 |
| 219 | Campylobacter spp | TCAAGATGGACAAAGTTTAAAAACAA | 149 | GAAATTCTTCCTCCTGTTTCAAAG | 168 |
| 220 | Campylobacter spp | TCAAGATGGACAAAGTTTAAAAACAA | 149 | CTGTTTCAAAGCGTGTTAAACCTA | 169 |
| 221 | Campylobacter spp | TCAAGATGGACAAAGTTTAAAAACAA | 149 | ATGAAATTCTTCCTCCTGTTTCAA | 170 |
| 222 | Campylobacter spp | TCAAGATGGACAAAGTTTAAAAACAA | 149 | CCGATTTGAAATTCTTGATTGATA | 171 |
| 223 | Campylobacter spp | CAAGATGGACAAAGTTTAAAAACAAG | 172 | TTTCAAAGCGTGTTAAACCTATCTTA | 152 |
| 224 | Campylobacter spp | TCAAGATGGACAAAGTTTAAAAACAA | 149 | ATTGCACTTCACCACTAGATGAAA | 173 |
| 225 | Campylobacter spp | TCAAGATGGACAAAGTTTAAAAACAA | 149 | AAATTGCACTTCACCACTAGATGA | 174 |
| 226 | Campylobacter spp | TCAAGATGGACAAAGTTTAAAAACAA | 149 | ACCGATTTGAAATTCTTGATTGAT | 175 |
| 227 | Campylobacter spp | ATTATCAAGATAGGACGCACTCATT | 176 | AAAGCATGAAATTTATTTGGACTTG | 177 |
| 228 | Campylobacter spp | ATTATCAAGATAGGACGCACTCATT | 176 | AAGCATGAAATTTATTTGGACTTGA | 178 |
| 229 | Campylobacter spp | ATTATCAAGATAGGACGCACTCATT | 176 | AGCATGAAATTTATTTGGACTTGAA | 179 |
| 230 | Campylobacter spp | TCAAGATGGACAAAGTTTAAAAACAA | 149 | TTAGAAGATTGAGTTGCTCCTATGG | 180 |
| 231 | Campylobacter spp | TCAAGATGGACAAAGTTTAAAAACAA | 149 | TAGAAGATTGAGTTGCTCCTATGGT | 181 |
| 232 | Campylobacter spp | TCAAGATGGACAAAGTTTAAAAACAA | 149 | AGAAGATTGAGTTGCTCCTATGGTA | 182 |
| 233 | Campylobacter spp | GTAAGTTCTATCGCGGGAACTACTA | 183 | ACCAAAAGTGCTATTTGAGAATGAG | 184 |
| 234 | Campylobacter spp | TCAAGATGGACAAAGTTTAAAAACAA | 149 | TCACCACTAGTTGAAATTCTTCCTC | 185 |
| 235 | Campylobacter spp | TCAAGATGGACAAAGTTTAAAAACAA | 149 | TTCACCACTAGTTGAAATTCTTCCT | 186 |
| 236 | Campylobacter spp | TCAAGATGGACAAAGTTTAAAAACAA | 149 | CTTCACCACTAGTTGAAATTCTTCC | 187 |
| 237 | Campylobacter spp | TCAAGATGGACAAAGTTTAAAAACAA | 149 | CTTTTATAGTTTGATTTGAACTCGCA | 188 |
| 238 | Campylobacter spp | GTAAGTTCTATCGCGGGAACTACTA | 183 | AAACCAAAAGTGCTATTTGAGAATG | 189 |
| 239 | Campylobacter spp | ATTATCAAGATAGGACGCACTCATT | 176 | TTAATCGCATCATGACTTGTTAAAG | 190 |
| 240 | Campylobacter spp | GTAAGTTCTATCGCGGGAACTACTA | 183 | AACCAAAAGTGCTATTTGAGAATGA | 191 |
| 241 | Campylobacter spp | TCAAGATGGACAAAGTTTAAAAACAA | 149 | ACCACTAGTTGAAATTCTTCCTCCT | 192 |
| 242 | Campylobacter spp | CTTTTTGTCATCGGTACTTCAGG | 193 | CCTACAAGCATTACAATAGCAATCA | 194 |
| 243 | Campylobacter spp | TCAAGATGGACAAAGTTTAAAAACAA | 149 | ACCTATCTTAGAAGACTGGGTTGCT | 195 |
| 244 | Campylobacter spp | CAAGATGGACAAAGTTTAAAAACAAG | 172 | CCTATCTTAGAAGATTGAGTTGCTCC | 155 |
| 245 | Campylobacter spp | TCAAGATGGACAAAGTTTAAAAACAA | 149 | GAACTAGCACCGATTTGAAATTCT | 196 |
| 246 | Campylobacter spp | TCAAGATGGACAAAGTTTAAAAACAA | 149 | AACTAGCACCGATTTGAAATTCTT | 197 |
| 247 | Campylobacter spp | TGCTATTGTAATGCTTGTAGGTTCTT | 198 | TCCAGCTATCATAGAAGCATATTCAC | 199 |
| 248 | Campylobacter spp | CAAGATGGACAAAGTTTAAAAACAAG | 172 | GTTTCAAAGCGTGTTAAACCTATCT | 158 |
| 249 | Campylobacter spp | CAAGATGGACAAAGTTTAAAAACAAG | 172 | TTCAAAGCGTGTTAAACCTATCTTA | 159 |
| 250 | Campylobacter spp | CAAGATGGACAAAGTTTAAAAACAAG | 172 | CACCACTAGATGAAATTCTTCCTCC | 160 |
| 251 | Canine adenovirus | AAAACTGACAATTCTGTTTCTCTGC | 200 | AGATATTCTTAAACCACTTCCAGCA | 201 |
| 252 | Canine adenovirus | AAGAGCTCTACCTGCAAATTATGAC | 202 | GAGGTAAAAGACAAAACCTTGTTGA | 203 |
| 253 | Canine adenovirus | CTCTAAGTGCTGGAAGTGGTTTAAG | 204 | GGCATATAAAGCACCTAATTACTGG | 205 |
| 254 | Canine adenovirus | TCCCTCCCACATAGATATAAAAGAC | 206 | AAAGGGTTAAGATATACCTCTGGCA | 207 |
| 255 | Canine adenovirus | AGGTACTTACTGACCTTCATGCAAC | 208 | AAACTCTTCAGCAAAGCTTAACAAA | 209 |
| 256 | Canine adenovirus | TAATTCTAAGATTAGGGTTACGGGG | 210 | AACAAGGTGTTTCTTTCACTAATGC | 211 |
| 257 | Canine adenovirus | TAATTCTAAGATTAGGGTTACGGGG | 210 | GCAATCAAAAATAACATTGGAAAAC | 212 |
| 258 | Canine adenovirus | CACTTGCACTGATACACCTATGAAC | 213 | GACACACATAGAAAATGTTGACTGC | 214 |
| 259 | Canine adenovirus | TTCGTAGAGAAACTGAGACCACTCT | 215 | GACGTCATACCATTATAAGCCAATC | 216 |
| 260 | Canine adenovirus | TTGGTTACCTATACTGGAGACCTTC | 217 | AAACAGAGTCCATTCCATAAAACTG | 218 |
| 261 | Canine adenovirus | GTACACTGATTCTGAGCTGTAAGCA | 219 | AGAGTGGTCTCAGTTTCTCTACGAA | 220 |
| 262 | Canine adenovirus | CCTGCAAATTATGACCCTGTATATC | 221 | TATGTAATGGGGAGGTAAAAGACAA | 222 |
| 263 | Canine adenovirus | GTTTCAAAGCAGTTTGTCTTCTCTC | 223 | ATAATTACTGGCTGGTGCTTAAGTG | 224 |
| 264 | Canine adenovirus | AGGTACTTACTGACCTTCATGCAAC | 208 | AAGAAACACAGAAACTACCTGGATG | 225 |
| 265 | Canine adenovirus | CTCAAAACCAAGCTGAGAGTAAAAG | 226 | TTTGTGTAAAGGGCATTATTCATTT | 227 |
| 266 | Canine adenovirus | CCTGCAAATTATGACCCTGTATATC | 221 | TTATGTAATGGGGAGGTAAAAGACA | 228 |
| 267 | Canine adenovirus | AAAATGAATAATGCCCTTTACACAA | 229 | GCTATAAGCTGGCACTATGTGGTAT | 230 |
| 268 | Canine adenovirus | GAGCTAAGAGCAGGTAGTATGGACC | 231 | AAGGCCAACTGTCTACCTCTATTTT | 232 |
| 269 | Canine adenovirus | AAATGAATAATGCCCTTTACACAAA | 233 | GCTATAAGCTGGCACTATGTGGTAT | 230 |
| 270 | Canine adenovirus | CAACTTTGATACTTCCACTTGTGTG | 234 | GGATGATTCAGTGAGACCTTTTCTA | 235 |
| 271 | Canine adenovirus | TTTGATATCTATGCTCATCAGACCA | 236 | GATCAGAAGATGTTAGCAACCCTTA | 237 |
| 272 | Canine adenovirus | AAGTGCTCCCATTATTATGACTGAG | 238 | AGTGGAAGTATCAAAGTTGGGAAG | 239 |
| 273 | Canine adenovirus | TATACCTTAACCCTTTTTCAGGACC | 240 | GAGTGGTGGGAACACTTAACTTCTA | 241 |
| 274 | Canine adenovirus | CCTGCAAATTATGACCCTGTATATC | 221 | TTTAATGGCACCTAACGTAGAAAAG | 242 |
| 275 | Canine adenovirus | CTTTTCTACGTTAGGTGCCATTAAA | 243 | TTATGTAATGGGGAGGTAAAAGACA | 228 |
| 276 | Canine adenovirus | TATCTATGCTCATCAGACCAATGAA | 244 | GATCAGAAGATGTTAGCAACCCTTA | 237 |
| 277 | Canine adenovirus | CACTTGCACTGATACACCTATGAAC | 213 | AAATGTTGACTGCTTAGGATGAAAC | 245 |
| 278 | Canine adenovirus | TATCTATGCTCATCAGACCAATGAA | 244 | AGATGTTAGCAACCCTTAATAACCC | 246 |
| 279 | Canine adenovirus | TGCTGCTGTAAAGTGTCTCTAAATG | 247 | ATGCTAGTGTTTCAATTACTGGGAG | 248 |
| 280 | Canine adenovirus | TTGAATCTGTTAAAACTTACAGGGC | 249 | TAGCCCTAAGTACAGCACAATTACC | 250 |
| 281 | Canine adenovirus | TAGAAAAGGTCTCACTGAATCATCC | 251 | GTCTTTTTCAACAGTGATTTGTCCT | 252 |
| 282 | Canine adenovirus | CATAGAAAGCAGTTCCTCAAAAGAG | 253 | AAGAAACACAGAAACTACCTGGATG | 225 |
| 283 | Canine adenovirus | CTGTGAAGACTTACAAAGCTAAGCC | 254 | GCAATCAAAAATAACATTGGAAAAC | 212 |
| 284 | Canine adenovirus | CATAGAAAGCAGTTCCTCAAAAGAG | 253 | CAAGAAACACAGAAACTACCTGGAT | 255 |
| 285 | Canine adenovirus | CTGTGAAGACTTACAAAGCTAAGCC | 254 | ATCAACATTCAAAACCTCAAACATT | 256 |
| 286 | Canine adenovirus | CCTGCAAATTATGACCCTGTATATC | 221 | GAGGTAAAAGACAAAACCTTGTTGA | 203 |
| 287 | Canine adenovirus | CAGAAGGAAGAAACAGTATCCGTTA | 257 | TGTACTCTGTCACATTAGGCATGTT | 258 |
| 288 | Canine adenovirus | CTTTTCTACGTTAGGTGCCATTAAA | 243 | GAGGTAAAAGACAAAACCTTGTTGA | 203 |
| 289 | Canine adenovirus | CAGAAGGAAGAAACAGTATCCGTTA | 257 | AACATGTACTCTGTCACATTAGGCA | 259 |
| 290 | Canine adenovirus | CTCCCAGTAATTGAAACACTAGCAT | 260 | TTTTCCTAATAAAAGACATTTCCCC | 261 |
| 291 | Canine adenovirus | TTGAATCTGTTAAAACTTACAGGGC | 249 | CGGAATAACATCTACATTCAAAACC | 262 |
| 292 | Canine adenovirus | ATGACCAGGATGTTAGGATTATTGA | 263 | TTTAAAGGCATCAGGCAAGTATAAG | 264 |
| 293 | Canine adenovirus | AGCCATAGTACATAACTTAGGCGTG | 265 | TGTACACTTGCTTGAGCTTACTGTC | 266 |
| 294 | Canine adenovirus | CATTTCTACTAGCTGCTCTTTTTGG | 267 | CTAAAACGCACGTTACTTACACCTT | 268 |
| 295 | Canine adenovirus | CCATTTCTACTAGCTGCTCTTTTTG | 269 | CTAAAACGCACGTTACTTACACCTT | 268 |
| 296 | Canine adenovirus | CATTTCTACTAGCTGCTCTTTTTGG | 267 | AAACGCACGTTACTTACACCTTTAC | 270 |
| 297 | Canine adenovirus | TTTGATATCTATGCTCATCAGACCA | 236 | AGATGTTAGCAACCCTTAATAACCC | 246 |
| 298 | Canine adenovirus | TTGAATCTGTTAAAACTTACAGGGC | 249 | AAACCAATTGACGGAATAACATCTA | 271 |
| 299 | Canine adenovirus | TAGGTCATTTAACAGACTGTCTCCC | 272 | AAGAAACACAGAAACTACCTGGATG | 225 |
| 300 | Canine adenovirus | CCATTTCTACTAGCTGCTCTTTTTG | 269 | AAACGCACGTTACTTACACCTTTAC | 270 |
| 301 | Canine coronavirus | ATGATCCTAAGACTGGACAATTCCT | 273 | CTCATCAATAATCTCAACCTGTGTG | 274 |
| 302 | Canine coronavirus | CCAGATATGTAATGTTCGGCTTTAG | 275 | ACGTATTTTGGTAAATTGTCGATGT | 276 |
| 303 | Canine coronavirus | GTGGTATGAACATCGACAATTTACC | 277 | GTTGAGTAATCACCAGCTTTAGATTTT | 278 |
| 304 | Canine coronavirus | GGTGGTATGAACATCGACAATTTAC | 279 | GTTGAGTAATCACCAGCTTTAGATTTT | 278 |
| 305 | Canine coronavirus | AACATTGCTTTCAGGGAATTTGTAT | 280 | GTTGAGTAATCACCAGCTTTAGATTTT | 278 |
| 306 | Canine coronavirus | TAACATTGCTTTCAGGGAATTTGTAT | 281 | GATTTTACATAGTAAGCCCATCCTGT | 282 |
| 307 | Canine coronavirus | TAACATTGCTTTCAGGGAATTTGTAT | 281 | AGATTTTACATAGTAAGCCCATCCTG | 283 |
| 308 | Canine coronavirus | AACATTGCTTTCAGGGAATTTGTAT | 280 | TAGATTTTACATAGTAAGCCCATCCTG | 284 |
| 309 | Canine coronavirus | GTGGTATGAACATCGACAATTTACC | 277 | TTGAGTAATCACCAGCTTTAGATTTT | 285 |
| 310 | Canine coronavirus | GGTGGTATGAACATCGACAATTTAC | 279 | TTGAGTAATCACCAGCTTTAGATTTT | 285 |
| 311 | Canine coronavirus | TGGTATGAACATCGACAATTTACC | 286 | GTTGAGTAATCACCAGCTTTAGATTTT | 278 |
| 312 | Canine coronavirus | AACATTGCTTTCAGGGAATTTGTAT | 280 | TTGAGTAATCACCAGCTTTAGATTTT | 285 |
| 313 | Canine coronavirus | ACATTGCTTTCAGGGAATTTGTAT | 287 | GTTGAGTAATCACCAGCTTTAGATTTT | 278 |
| 314 | Canine coronavirus | AACATTGCTTTCAGGGAATTTGTAT | 280 | GCTTTAGATTTTACATAGTAAGCCCA | 288 |
| 315 | Canine coronavirus | GGTGTCACTCTAACATTGCTTTCAG | 289 | TAGATTTTACATAGTAAGCCCATCCTG | 284 |
| 316 | Canine coronavirus | GTGTCACTCTAACATTGCTTTCAGG | 290 | TTGAGTAATCACCAGCTTTAGATTTT | 285 |
| 317 | Canine coronavirus | GGTGTCACTCTAACATTGCTTTCAG | 289 | TTGAGTAATCACCAGCTTTAGATTTT | 285 |
| 318 | Canine coronavirus | TAACATTGCTTTCAGGGAATTTGTAT | 281 | ATTTTACATAGTAAGCCCATCCTGTC | 291 |
| 319 | Canine coronavirus | AACATTGCTTTCAGGGAATTTGTAT | 280 | GATTTTACATAGTAAGCCCATCCAGT | 292 |
| 320 | Canine coronavirus | AACATTGCTTTCAGGGAATTTGTAT | 280 | GATTTTACATAGTAAGCCCATCCTGT | 282 |
| 321 | Canine coronavirus | AACATTGCTTTCAGGGAATTTGTAT | 280 | AGATTTTACATAGTAAGCCCATCCAG | 293 |
| 322 | Canine coronavirus | AACATTGCTTTCAGGGAATTTGTAT | 280 | AGATTTTACATAGTAAGCCCATCCTG | 283 |
| 323 | Canine coronavirus | AACATTGCTTTCAGGGAATTTGTAT | 280 | TAGATTTTACATAGTAAGCCCATCCA | 294 |
| 324 | Canine coronavirus | TAACATTGCTTTCAGGGAATTTGTAT | 281 | GTTGCACTACTTGCTTTCAATTCT | 295 |
| 325 | Canine coronavirus | TAACATTGCTTTCAGGGAATTTGTAT | 281 | GATTTTACATAGTAAGCCCATCCAG | 296 |
| 326 | Canine coronavirus | TAACATTGCTTTCAGGGAATTTGTAT | 281 | AGATTTTACATAGTAAGCCCATCCA | 297 |
| 327 | Canine coronavirus | TAACATTGCTTTCAGGGAATTTGTAT | 281 | GATTTTACATAGTAAGCCCATCCTG | 298 |
| 328 | Canine coronavirus | ACATTGCTTTCAGGGAATTTGTAT | 287 | TAGATTTTACATAGTAAGCCCATCCTG | 284 |
| 329 | Canine coronavirus | TAACATTGCTTTCAGGGAATTTGTAT | 281 | ATTTTACATAGTAAGCCCATCCTGT | 299 |
| 330 | Canine coronavirus | CTAACATTGCTTTCAGGGAATTTGT | 300 | GATTTTACATAGTAAGCCCATCCAGT | 292 |
| 331 | Canine coronavirus | CTAACATTGCTTTCAGGGAATTTGT | 300 | GATTTTACATAGTAAGCCCATCCTGT | 282 |
| 332 | Canine coronavirus | CTAACATTGCTTTCAGGGAATTTGT | 300 | AGATTTTACATAGTAAGCCCATCCTG | 283 |
| 333 | Canine coronavirus | CTAACATTGCTTTCAGGGAATTTGT | 300 | AGATTTTACATAGTAAGCCCATCCAG | 293 |
| 334 | Canine coronavirus | AACATTGCTTTCAGGGAATTTGTAT | 280 | CTTTAGATTTTACATGGTAAGCCCAT | 301 |
| 335 | Canine coronavirus | TAACATTGCTTTCAGGGAATTTGTAT | 281 | CTTTAGATTTTACATGGTAAGCCCA | 302 |
| 336 | Canine coronavirus | TAACATTGCTTTCAGGGAATTTGTAT | 281 | TTAGATTTTACATGGTAAGCCCATC | 303 |
| 337 | Canine coronavirus | TAACATTGCTTTCAGGGAATTTGTAT | 281 | GCTTTAGATTTTACATGGTAAGCCC | 304 |
| 338 | Canine coronavirus | TAACATTGCTTTCAGGGAATTTGTAT | 281 | TTGCACTACTTGCTTTCAATTTCTT | 305 |
| 339 | Canine coronavirus | TAACATTGCTTTCAGGGAATTTGTAT | 281 | TTTAGATTTTACATGGTAAGCCCAT | 306 |
| 340 | Canine coronavirus | AACATTGCTTTCAGGGAATTTGTAT | 280 | TTTAGATTTTACATGGTAAGCCCATC | 307 |
| 341 | Canine coronavirus | GTGTCACTCTAACATTGCTTTCAGG | 290 | GATTTTACATAGTAAGCCCATCCAGT | 292 |
| 342 | Canine coronavirus | GTGTCACTCTAACATTGCTTTCAGG | 290 | GATTTTACATAGTAAGCCCATCCTGT | 282 |
| 343 | Canine coronavirus | GGTGTCACTCTAACATTGCTTTCAG | 289 | GATTTTACATAGTAAGCCCATCCAGT | 292 |
| 344 | Canine coronavirus | GGTGTCACTCTAACATTGCTTTCAG | 289 | GATTTTACATAGTAAGCCCATCCTGT | 282 |
| 345 | Canine coronavirus | GTGTCACTCTAACATTGCTTTCAGG | 290 | AGATTTTACATAGTAAGCCCATCCAG | 293 |
| 346 | Canine coronavirus | GGTGTCACTCTAACATTGCTTTCAG | 289 | AGATTTTACATAGTAAGCCCATCCAG | 293 |
| 347 | Canine coronavirus | GGTGTCACTCTAACATTGCTTTCAG | 289 | AGATTTTACATAGTAAGCCCATCCTG | 283 |
| 348 | Canine coronavirus | ACATTGCTTTCAGGGAATTTGTAT | 287 | TTGAGTAATCACCAGCTTTAGATTTT | 285 |
| 349 | Canine coronavirus | AAGGGTTCAAAATTACAGGTGGTAT | 308 | CTTAGATTTTACATAGTAAGCCCATCC | 309 |
| 350 | Canine coronavirus | ACATTGCTTTCAGGGAATTTGTAT | 287 | GCTTTAGATTTTACATAGTAAGCCCA | 288 |
| 351 | Canine distemper virus | AATTGTACCAAAGCAGAATTAGGTG | 310 | AAGGCTAGTTCTCAGAGATTGGATT | 311 |
| 352 | Canine distemper virus | AAAGATCCGAAGTCACTAATCAACA | 312 | TCTTAGTAAGCATTCTCATCTTGGC | 313 |
| 353 | Canine distemper virus | AAGAGGTTAAGGGAATCGAAGATG | 314 | GAAAGAATATCCCCAGTTAGATGAAG | 315 |
| 354 | Canine distemper virus | AAGAGGTTAAGGGAATCGAAGATG | 314 | CGAAAGAATATCCCCAGTTAGATGA | 316 |
| 355 | Canine distemper virus | AAGAGGTTAAGGGAATCGAAGATG | 314 | ATATCCCCAGTTAGATGAAGCATTT | 317 |
| 356 | Canine distemper virus | AAGAGGTTAAGGGAATCGAAGATG | 314 | GAAAGAATATCCCCAGTTAGATGAA | 318 |
| 357 | Canine distemper virus | AAGAGGTTAAGGGAATCGAAGATG | 314 | GAAAGAATATCCCCAGTTAGATGAAT | 319 |
| 358 | Canine distemper virus | AAGAGGTTAAGGGAATCGAAGATG | 314 | AAGAATATCCCCAGTTAGATGAAGC | 320 |
| 359 | Canine distemper virus | AAGAGGTTAAGGGAATCGAAGATG | 314 | AATATCCCCAGTTAGATGAAGCATT | 321 |
| 360 | Canine distemper virus | AAGAGGTTAAGGGAATCGAAGATG | 314 | GAATATCCCCAGTTAGATGAAGCAT | 322 |
| 361 | Canine distemper virus | AAGAGGTTAAGGGAATCGAAGATG | 314 | AGAATATCCCCAGTTAGATGAAGCA | 323 |
| 362 | Canine distemper virus | AAGAGGTTAAGGGAATCGAAGATG | 314 | AAAGAATATCCCCAGTTAGATGAAG | 324 |
| 363 | Canine distemper virus | AAGAGGTTAAGGGAATCGAAGATG | 314 | TTTTCTGAATCCCTACATTCTGCT | 325 |
| 364 | Canine distemper virus | CTTCATCTAACTGGGGATATTCTTTC | 326 | GATCCAGCAACTATCTCCATTCC | 327 |
| 365 | Canine distemper virus | AAGAGGTTAAGGGAATCGAAGAT | 328 | ATATCCCCAGTTAGATGAAGCATTT | 317 |
| 366 | Canine distemper virus | AAGAGGTTAAGGGAATCGAAGAT | 328 | GAAAGAATATCCCCAGTTAGATGAA | 318 |
| 367 | Canine distemper virus | AAGAGGTTAAGGGAATCGAAGAT | 328 | AAGAATATCCCCAGTTAGATGAAGC | 320 |
| 368 | Canine distemper virus | AAGAGGTTAAGGGAATCGAAGAT | 328 | AATATCCCCAGTTAGATGAAGCATT | 321 |
| 369 | Canine distemper virus | AAGAGGTTAAGGGAATCGAAGAT | 328 | GAATATCCCCAGTTAGATGAAGCAT | 322 |
| 370 | Canine distemper virus | AAGAGGTTAAGGGAATCGAAGATG | 314 | CGAAAGAATATCCCCAGTTAGATG | 329 |
| 371 | Canine distemper virus | AAGAGGTTAAGGGAATCGAAGATG | 314 | CCGAAAGAATATCCCCAGTTAGAT | 330 |
| 372 | Canine distemper virus | AAGAGGTTAAGGGAATCGAAGATG | 314 | GAATATCCCCAGTTAGATGAAGCA | 331 |
| 373 | Canine distemper virus | AAGAGGTTAAGGGAATCGAAGATG | 314 | TATCCCCAGTTAGATGAAGCATTT | 332 |
| 374 | Canine distemper virus | AAGAGGTTAAGGGAATCGAAGATG | 314 | ATATCCCCAGTTAGATGAAGCATT | 333 |
| 375 | Canine distemper virus | AAGAGGTTAAGGGAATCGAAGATG | 314 | AATATCCCCAGTTAGATGAAGCAT | 334 |
| 376 | Canine distemper virus | AAGAGGTTAAGGGAATCGAAGATG | 314 | CTACATTTCTGCTTGTCTTGAGCAG | 335 |
| 377 | Canine distemper virus | AAGAGGTTAAGGGAATCGAAGATG | 314 | AGAATATCCCCAGTTAGATGAAGC | 336 |
| 378 | Canine distemper virus | AAGAGGTTAAGGGAATCGAAGATG | 314 | CTGAATCCCTACATTTCTGCTTGT | 337 |
| 379 | Canine distemper virus | AAGAGGTTAAGGGAATCGAAGATG | 314 | CTGAATCCCTACATTTCTGCTTGTT | 338 |
| 380 | Canine distemper virus | AAGAGGTTAAGGGAATCGAAGATG | 314 | TTTCTGAATCCCTACATTTCTGCT | 339 |
| 381 | Canine distemper virus | TTCATCTAACTGGGGATATTCTTTC | 340 | GATCCAGCAACTATCTCCATTCC | 327 |
| 382 | Canine distemper virus | GCTTCATCTAACTGGGGATATTCTT | 341 | GATCCAGCAACTATCTCCATTCC | 327 |
| 383 | Canine distemper virus | AAGAGGTTAAGGGAATCGAAGATG | 314 | ATTATGTGGGAACTGCAGAGTCTT | 342 |
| 384 | Canine distemper virus | AAGAGGTTAAGGGAATCGAAGATG | 314 | GAAAGAATGTCCCCAGTTAGATGAA | 343 |
| 385 | Canine distemper virus | AAGAGGTTAAGGGAATCGAAGATG | 314 | AAAGAATGTCCCCAGTTAGATGAAG | 344 |
| 386 | Canine distemper virus | AAGAGGTTAAGGGAATCGAAGATG | 314 | GAATCCCTACATTTCTGCTTGTTCT | 345 |
| 387 | Canine distemper virus | AAGAGGTTAAGGGAATCGAAGATG | 314 | TACATTTCTGCTTGTCTTGAGCAG | 346 |
| 388 | Canine distemper virus | AAGAGGTTAAGGGAATCGAAGATG | 314 | AATCCCTACATTTCTGCTTGTTCTG | 347 |
| 389 | Canine distemper virus | AAGAGGTTAAGGGAATCGAAGATG | 314 | ATCCCCAGTTAGATGAAGCATTT | 348 |
| 390 | Canine distemper virus | AAGAGGTTAAGGGAATCGAAGATG | 314 | TGAATCCCTACATTTCTGCTTGT | 349 |
| 391 | Canine distemper virus | AAGAGGTTAAGGGAATCGAAGATG | 314 | CTGAATCCCTACATTTCTGCTTG | 350 |
| 392 | Canine distemper virus | AGAGGTTAAGGGAATCGAAGATG | 351 | GAAAGAATATCCCCAGTTAGATGAAG | 315 |
| 393 | Canine distemper virus | AGAGGTTAAGGGAATCGAAGATG | 351 | AAAGAATATCCCCAGTTAGATGAAGC | 352 |
| 394 | Canine distemper virus | CATCTAACTGGGGATATTCTTTCG | 353 | GATCCAGCAACTATCTCCATTCC | 327 |
| 395 | Canine distemper virus | AAGAGGTTAAGGGAATCGAAGATG | 314 | AGTCTTTCCATCCCTTTTCTGAAT | 354 |
| 396 | Canine distemper virus | TACAGTCAGTAGGGTCAGGTAGGAG | 355 | CAAGGCTAGTTCTCAGAGATTGGAT | 356 |
| 397 | Canine distemper virus | CTACAGTCAGTAGGGTCAGGTAGGA | 357 | CAAGGCTAGTTCTCAGAGATTGGAT | 356 |
| 398 | Canine distemper virus | AAGAGGTTAAGGGAATCGAAGATG | 314 | AAAGAATGTCCCCAGTTAGATGAA | 358 |
| 399 | Canine distemper virus | AAGAGGTTAAGGGAATCGAAGATG | 314 | GAAAGAATGTCCCCAGTTAGATGA | 359 |
| 400 | Canine distemper virus | AAGAGGTTAAGGGAATCGAAGATG | 314 | AAGAATGTCCCCAGTTAGATGAAG | 360 |
| 401 | Canine herpesvirus | ATATACCTCATCACACCCTTCTTCC | 361 | TTTTAAGCAATTCAGAATCCTCATC | 362 |
| 402 | Canine herpesvirus | AACATGCGAGATTATGTCAATTTTT | 363 | ACAGCTGATGAATTTGACTTCACTT | 364 |
| 403 | Canine herpesvirus | CTCTTTTAGAGTCCCAATATCGTCC | 365 | CCAATAAAATATCAAATTGTGCAGC | 366 |
| 404 | Canine herpesvirus | CAACACCAATAAATAATACCCCAAA | 367 | CCTCTTGCAAGTGAATAAAAGTTGT | 368 |
| 405 | Canine herpesvirus | ACTGGATATAAATGGGTAGACCCTC | 369 | AGAAATCAAAGCAACCATATCACAT | 370 |
| 406 | Canine herpesvirus | TTTGTGAAACTTCCATTTTCATATC | 371 | GAAGAAGGTACTTCCATAAGCTGAG | 372 |
| 407 | Canine herpesvirus | ACTCACTTGTTTTTGGAAACGATAG | 373 | ATCGTTTATGATTAGGATTTGTGGA | 374 |
| 408 | Canine herpesvirus | TCATCACCATTAAATGAAACAAATG | 375 | CTAAAACAATAAATCCACAAATCCG | 376 |
| 409 | Canine herpesvirus | AATTGAACTTAATCTTGAATCGGAA | 377 | ATCAGTTGTCCAGTTTTTACCTTCA | 378 |
| 410 | Canine herpesvirus | ACTCACTTGTTTTTGGAAACGATAG | 373 | TCGTTTATGATTAGGATTTGTGGAT | 379 |
| 411 | Canine herpesvirus | GATGAGGATTCTGAATTGCTTAAAA | 380 | CCCCACATGTCAGTTCTATATTTTC | 381 |
| 412 | Canine herpesvirus | GATGAGGATTCTGAATTGCTTAAAA | 380 | TTTCCCCACATGTCAGTTCTATATT | 382 |
| 413 | Canine herpesvirus | AAACCATCGTTAGTTGGTTACACAT | 383 | CTCGTGGTAATTGAACCATAAAATC | 384 |
| 414 | Canine herpesvirus | GATGAGGATTCTGAATTGCTTAAAA | 380 | TTCCCCACATGTCAGTTCTATATTT | 385 |
| 415 | Canine herpesvirus | ACCATCGTTAGTTGGTTACACATTT | 386 | CTCGTGGTAATTGAACCATAAAATC | 384 |
| 416 | Canine herpesvirus | AGTTCTATCAAAGGATGCGTACAAC | 387 | GACGATTATATCGTGTAAATGGAGG | 388 |
| 417 | Canine herpesvirus | GTTCTATCAAAGGATGCGTACAACT | 389 | GACGATTATATCGTGTAAATGGAGG | 388 |
| 418 | Canine herpesvirus | AAACCATCGTTAGTTGGTTACACAT | 383 | TCTCGTGGTAATTGAACCATAAAAT | 390 |
| 419 | Canine herpesvirus | GTGTTTTGTTGGAAATTACAAATCC | 391 | TAGGTGTAATAGTTTGGGAAGAAGG | 392 |
| 420 | Canine herpesvirus | GTTCTATCAAAGGATGCGTACAACT | 389 | CGATTATATCGTGTAAATGGAGGTC | 393 |
| 421 | Canine herpesvirus | AATATAGAACTGACATGTGGGGAAA | 394 | TAAAGATTGGAGAAATGGAGACTTG | 395 |
| 422 | Canine herpesvirus | GAAAATATAGAACTGACATGTGGGG | 396 | TAAAGATTGGAGAAATGGAGACTTG | 395 |
| 423 | Canine herpesvirus | AGAATATGGGAGTGGTTCACATAAA | 397 | TATGAAGAAATTCCTGAACCAGAAG | 398 |
| 424 | Canine herpesvirus | AAAATATAGAACTGACATGTGGGGA | 399 | TAAAGATTGGAGAAATGGAGACTTG | 395 |
| 425 | Canine herpesvirus | AGTTCTATCAAAGGATGCGTACAAC | 387 | CGATTATATCGTGTAAATGGAGGTC | 393 |
| 426 | Canine herpesvirus | AAATATAGAACTGACATGTGGGGAA | 400 | TAAAGATTGGAGAAATGGAGACTTG | 395 |
| 427 | Canine herpesvirus | ATATAGAACTGACATGTGGGGAAAA | 401 | TAAAGATTGGAGAAATGGAGACTTG | 395 |
| 428 | Canine herpesvirus | AAGAATATGGGAGTGGTTCACATAA | 402 | TATGAAGAAATTCCTGAACCAGAAG | 398 |
| 429 | Canine herpesvirus | TGGAAATTACAAATCCTAAACCAAA | 403 | TTCCATGCAAACCTAGTAAAAGTTC | 404 |
| 430 | Canine herpesvirus | GGACTATTGAAACTAATGCTCCTGA | 405 | GACGATTATATCGTGTAAATGGAGG | 388 |
| 431 | Canine herpesvirus | CTAATGTTTTGTTTGAGGAGGAAGA | 406 | GACGATTATATCGTGTAAATGGAGG | 388 |
| 432 | Canine herpesvirus | GGACTATTGAAACTAATGCTCCTGA | 405 | CGATTATATCGTGTAAATGGAGGTC | 393 |
| 433 | Canine herpesvirus | AGAATATGGGAGTGGTTCACATAAA | 397 | AAACAATAACCAAAAATGAAAACGA | 407 |
| 434 | Canine herpesvirus | AAGAATATGGGAGTGGTTCACATAA | 402 | AAACAATAACCAAAAATGAAAACGA | 407 |
| 435 | Canine herpesvirus | TGTAAATGTATCTTCACGACCAGAA | 408 | CTATCGTTTCCAAAAACAAGTGAGT | 409 |
| 436 | Canine herpesvirus | TGTAAATGTATCTTCACGACCAGAA | 408 | ACTATCGTTTCCAAAAACAAGTGAG | 410 |
| 437 | Canine herpesvirus | AACCATCGTTAGTTGGTTACACATT | 411 | TTACACCATTCCATAAAAGTTGGTT | 412 |
| 438 | Canine herpesvirus | ACCATCGTTAGTTGGTTACACATTT | 386 | TTACACCATTCCATAAAAGTTGGTT | 412 |
| 439 | Canine herpesvirus | AGAATATGGGAGTGGTTCACATAAA | 397 | TGAAAACGATGGTATGTCTTCTACA | 413 |
| 440 | Canine herpesvirus | AAACCATCGTTAGTTGGTTACACAT | 383 | TTACACCATTCCATAAAAGTTGGTT | 412 |
| 441 | Canine herpesvirus | TTGGAAATTACAAATCCTAAACCAA | 414 | TTCCATGCAAACCTAGTAAAAGTTC | 404 |
| 442 | Canine herpesvirus | AAGAATATGGGAGTGGTTCACATAA | 402 | TGAAAACGATGGTATGTCTTCTACA | 413 |
| 443 | Canine herpesvirus | CGGATTTGTGGATTTATTGTTTTAG | 415 | CCTCCGTTGGTTTTAAAAATTTACT | 416 |
| 444 | Canine herpesvirus | TGGAAATTACAAATCCTAAACCAAA | 403 | AGTATGACATATCGGTTCGTGTTTT | 417 |
| 445 | Canine herpesvirus | CTGCAGGTTCTCAAAGTTTATTGAT | 418 | ATGTGTATTTTCAGTAGGTGGTGGT | 419 |
| 446 | Canine herpesvirus | AACCATCGTTAGTTGGTTACACATT | 411 | GTCAACTTTTGATGCTCCTACAGAT | 420 |
| 447 | Canine herpesvirus | ACCATCGTTAGTTGGTTACACATTT | 386 | GTCAACTTTTGATGCTCCTACAGAT | 420 |
| 448 | Canine herpesvirus | TCATCACCATTAAATGAAACAAATG | 375 | TAAAACAATAAATCCACAAATCCGT | 421 |
| 449 | Canine herpesvirus | AAACCATCGTTAGTTGGTTACACAT | 383 | GTCAACTTTTGATGCTCCTACAGAT | 420 |
| 450 | Canine herpesvirus | TGTTTTGTTGGAAATTACAAATCCT | 422 | TTCCATGCAAACCTAGTAAAAGTTC | 404 |
| 451 | Canine oral papilloma | TGATCCTGAAAAAGAAAGACTTGTT | 423 | GTGCACCATCTAGTTAAAGACCAGT | 424 |
| | virus | | | | |
| 452 | Canine oral papilloma | AAAGTATCTCCTAATCAGTACCGGG | 425 | TAACACTTATACCTAATGGTTGCCC | 426 |
| | virus | | | | |
| 453 | Canine oral papilloma | ACCTTCTCTCTCACTAGCAAAACAA | 427 | AAGGAACTTATGGAGTATGCAACAA | 428 |
| | virus | | | | |
| 454 | Canine oral papilloma | ACTGTAGAAAACCAGTGGAATTCAG | 429 | TTGTTTTGCTAGTGAGAGAGAAGGT | 430 |
| | virus | | | | |
| 455 | Canine oral papilloma | ATGAATGCTGTTAATTTTGTTCCAT | 431 | TTGTTTTGCTAGTGAGAGAGAAGGT | 430 |
| | virus | | | | |
| 456 | Canine oral papilloma | AAAGCCATTTTTACTACGATGTCAG | 432 | AAGGCTTTATGTAACGTTGAACTTG | 433 |
| | virus | | | | |
| 457 | Canine oral papilloma | ATCAGCAAGTACAAGTTACTGACCC | 434 | CTGACATCGTAGTAAAAATGGCTTT | 435 |
| | virus | | | | |
| 458 | Canine oral papilloma | AATGATGTAGAAAACCCAAACAAAA | 436 | GCACCATCTAGTTAAAGACCAGTGT | 437 |
| | virus | | | | |
| 459 | Canine oral papilloma | ATGATGTAGAAAACCCAAACAAAAA | 438 | GTGCACCATCTAGTTAAAGACCAGT | 424 |
| | virus | | | | |
| 460 | Canine oral papilloma | AATGATGTAGAAAACCCAAACAAAA | 436 | GTGCACCATCTAGTTAAAGACCAGT | 424 |
| | virus | | | | |
| 461 | Canine oral papilloma | ATGATGTAGAAAACCCAAACAAAAA | 438 | GCACCATCTAGTTAAAGACCAGTGT | 437 |
| | virus | | | | |
| 462 | Canine oral papilloma | TGATGTAGAAAACCCAAACAAAAAT | 439 | GCACCATCTAGTTAAAGACCAGTGT | 437 |
| | virus | | | | |
| 463 | Canine oral papilloma | AAACATGTAAAAGACTGTGCAACAA | 440 | AACATAGACTTGCCTGTATTTGGAG | 441 |
| | virus | | | | |
| 464 | Canine oral papilloma | TGATGTAGAAAACCCAAACAAAAAT | 439 | GTGCACCATCTAGTTAAAGACCAGT | 424 |
| | virus | | | | |
| 465 | Canine oral papilloma | AAAAACATTATTGCAGGATCATTGT | 442 | GCAACTGCAGGACTTCTAGTTTTAG | 443 |
| | virus | | | | |
| 466 | Canine oral papilloma | AGAGGATCCTTTTACTCTACCTCCA | 444 | AGGGTTACTGTACTGATGCCTAGTG | 445 |
| | virus | | | | |
| 467 | Canine oral papilloma | AAACATGTAAAAGACTGTGCAACAA | 440 | TTTTAGGCCTACCTCTCAAAAATCT | 446 |
| | virus | | | | |
| 468 | Canine oral papilloma | ATATGGTGACAGTGAGGAAAACATT | 447 | TAGAATCTCATATTTCCCAGTCTCG | 448 |
| | virus | | | | |
| 469 | Canine oral papilloma | AGAAGAGAGGCAGGAAAAACATTAT | 449 | GCAACTGCAGGACTTCTAGTTTTAG | 443 |
| | virus | | | | |
| 470 | Canine oral papilloma | ATGCTGTTAATTTTGTTCCATTTGT | 450 | TTGTTTTGCTAGTGAGAGAGAAGGT | 430 |
| | virus | | | | |
| 471 | Canine oral papilloma | ATAGAAGAGAGGCAGGAAAAACATT | 451 | GCAACTGCAGGACTTCTAGTTTTAG | 443 |
| | virus | | | | |
| 472 | Canine oral papilloma | GTATAAGTGTTACGGGTCATCCAAC | 452 | GCACCATCTAGTTAAAGACCAGTGT | 437 |
| | virus | | | | |
| 473 | Canine oral papilloma | TAATTACTTCTCCACACCTAGTGGC | 453 | TATAGTTAAGGGAGTTCCTCGTGTG | 454 |
| | virus | | | | |
| 474 | Canine oral papilloma | ATGATGTAGAAAACCCAAACAAAAA | 438 | TATAATGCTGCAAAGCCTTAAAATC | 455 |
| | virus | | | | |
| 475 | Canine oral papilloma | AATGATGTAGAAAACCCAAACAAAA | 436 | TATAATGCTGCAAAGCCTTAAAATC | 455 |
| | virus | | | | |
| 476 | Canine oral papilloma | TGATGTAGAAAACCCAAACAAAAAT | 439 | TATAATGCTGCAAAGCCTTAAAATC | 455 |
| | virus | | | | |
| 477 | Canine oral papilloma | AAACATGTAAAAGACTGTGCAACAA | 440 | CAAAAATCTTTTAAAGTCTGCCAAA | 456 |
| | virus | | | | |
| 478 | Canine oral papilloma | GTCTTAAGCACGGATGAATATGTCT | 457 | GAATACCCGGTACTGATTAGGAGAT | 458 |
| | virus | | | | |
| 479 | Canine oral papilloma | TAATTACTTCTCCACACCTAGTGGC | 453 | ATAATTTCCTTCTTCTGGCTTGTCT | 459 |
| | virus | | | | |
| 480 | Canine oral papilloma | ACCTTCTCTCTCACTAGCAAAACAA | 427 | TTCAAAAGGAACTTATGGAGTATGC | 460 |
| | virus | | | | |
| 481 | Canine oral papilloma | AGACAAGCCAGAAGAAGGAAATTAT | 461 | TCATCTAAAGTACCAGATGGAGGAG | 462 |
| | virus | | | | |
| 482 | Canine oral papilloma | TAGCAGTGAACGTCTTCTTACTGTG | 463 | ATTTTTGTTTGGGTTTTCTACATCA | 464 |
| | virus | | | | |
| 483 | Canine oral papilloma | TAGCAGTGAACGTCTTCTTACTGTG | 463 | TTTTGTTTGGGTTTTCTACATCATT | 465 |
| | virus | | | | |
| 484 | Canine oral papilloma | TAGCAGTGAACGTCTTCTTACTGTG | 463 | TTTTTGTTTGGGTTTTCTACATCAT | 466 |
| | virus | | | | |
| 485 | Canine oral papilloma | ACTAGGCATCAGTACAGTAACCCTG | 467 | TTCAGATCTAGAGCCTTGTTTAGGA | 468 |
| | virus | | | | |
| 486 | Canine oral papilloma | TAGCAGTGAACGTCTTCTTACTGTG | 463 | GTTGGATGACCCGTAACACTTATAC | 469 |
| | virus | | | | |
| 487 | Canine oral papilloma | AGTTAAAGTGAACAACGACAAGTCC | 470 | GAAGTTTCACTTGCCTAATTGGATA | 471 |
| | virus | | | | |
| 488 | Canine oral papilloma | AAACATGTAAAAGACTGTGCAACAA | 440 | CTTTTAAAGTCTGCCAAAAACAAAA | 472 |
| | virus | | | | |
| 489 | Canine oral papilloma | GAGGATCCTTTTACTCTACCTCCAC | 473 | AGGGTTACTGTACTGATGCCTAGTG | 445 |
| | virus | | | | |
| 490 | Canine oral papilloma | ACCTGATATTTGAAAGAGATGTTGC | 474 | CTGACATCGTAGTAAAAATGGCTTT | 435 |
| | virus | | | | |
| 491 | Canine oral papilloma | GCAGTTTAAAGCATAAGCAAACAAT | 475 | ATATGATGGTATTTTCGCAGTGAAT | 476 |
| | virus | | | | |
| 492 | Canine oral papilloma | TCAACAAACGTTAGTCTCTTAGCCT | 477 | ATATGATGGTATTTTCGCAGTGAAT | 476 |
| | virus | | | | |
| 493 | Canine oral papilloma | GATTAAGTCATAAGCATAGGGGGTT | 478 | TATGTCCCAACACCTTACTACACCT | 479 |
| | virus | | | | |
| 494 | Canine oral papilloma | AGACAAGCCAGAAGAAGGAAATTAT | 461 | TGTGTCATCTAAAGTACCAGATGGA | 480 |
| | virus | | | | |
| 495 | Canine oral papilloma | TTAGATTTAAGTGACCCTGAAGACG | 481 | TGGCATAATAAAGTAGGACTTGCTC | 482 |
| | virus | | | | |
| 496 | Canine oral papilloma | AACCTGTAGAACATCTCTGCATTTC | 483 | GATTGCTTTTGACATTAAAGCTGAT | 484 |
| | virus | | | | |
| 497 | Canine oral papilloma | TTAGATTTAAGTGACCCTGAAGACG | 481 | TCTGGCATAATAAAGTAGGACTTGC | 485 |
| | virus | | | | |
| 498 | Canine oral papilloma | TAATTACTTCTCCACACCTAGTGGC | 453 | GAGGTAGGTTCTGTATGATGAAGGA | 486 |
| | virus | | | | |
| 499 | Canine oral papilloma | GAATGAATGCTGTTAATTTTGTTCC | 487 | TTGTTTTGCTAGTGAGAGAGAAGGT | 430 |
| | virus | | | | |
| 500 | Canine oral papilloma | ACTGAACAGTTAGACCAAACTCCAC | 488 | AGTCAGTAAACAATGACCACAACAA | 489 |
| | virus | | | | |
| 501 | Canine parvovirus | ACAGATCCAGATGATGTTCAATTTT | 490 | ACCACGTCTTTTATCTTGTTGAACT | 491 |
| 502 | Canine parvovirus | AGATATCCAGAAGGAGATTGGATTC | 492 | CATTTGGATAAACTGGTGGTACATT | 493 |
| 503 | Canine parvovirus | ATGTATTGCTACCAACAGATCCAAT | 494 | AATTAGGCGCAACTTTTACAAATAA | 495 |
| 504 | Canine parvovirus | ATGTATTGCTACCAACAGATCCAAT | 494 | AAATTAGGCGCAACTTTTACAAATA | 496 |
| 505 | Canine parvovirus | AAGATATCCAGAAGGAGATTGGATT | 497 | CATTTGGATAAACTGGTGGTACATT | 493 |
| 506 | Canine parvovirus | AGATATCCAGAAGGAGATTGGATTC | 492 | AATTAGGCGCAACTTTTACAAATAA | 495 |
| 507 | Canine parvovirus | AGATATCCAGAAGGAGATTGGATTC | 492 | AAATTAGGCGCAACTTTTACAAATA | 496 |
| 508 | Canine parvovirus | AAGATATCCAGAAGGAGATTGGATT | 497 | AATTAGGCGCAACTTTTACAAATAA | 495 |
| 509 | Canine parvovirus | AAGATATCCAGAAGGAGATTGGATT | 497 | AAATTAGGCGCAACTTTTACAAATA | 496 |
| 510 | Canine parvovirus | ACAGGAAGATATCCAGAAGGAGATT | 498 | CATTTGGATAAACTGGTGGTACATT | 493 |
| 511 | Canine parvovirus | GAAGATATCCAGAAGGAGATTGGAT | 499 | CATTTGGATAAACTGGTGGTACATT | 493 |
| 512 | Canine parvovirus | AATGTACCACCAGTTTATCCAAATG | 500 | TACCTTTCCACCAAAAATCTGAGTA | 501 |
| 513 | Canine parvovirus | CAAGATATGCATTTGGTAGACAACA | 502 | CATTTGGATAAACTGGTGGTACATT | 493 |
| 514 | Canine parvovirus | GAAGATATCCAGAAGGAGATTGGAT | 499 | AAATTAGGCGCAACTTTTACAAATA | 496 |
| 515 | Canine parvovirus | GAAGATATCCAGAAGGAGATTGGAT | 499 | AATTAGGCGCAACTTTTACAAATAA | 495 |
| 516 | Canine parvovirus | ATGTATTGCTACCAACAGATCCAAT | 494 | GTGCATTTACATGAAGTCTTGGTTT | 503 |
| 517 | Canine parvovirus | AATGTACCACCAGTTTATCCAAATG | 500 | TTTCCACCAAAAATCTGAGTAAGTT | 504 |
| 518 | Canine parvovirus | AGATATCCAGAAGGAGATTGGATTC | 492 | GTGCATTTACATGAAGTCTTGGTTT | 503 |
| 519 | Canine parvovirus | AAGATATCCAGAAGGAGATTGGATT | 497 | GTGCATTTACATGAAGTCTTGGTTT | 503 |
| 520 | Canine parvovirus | ATGTATTGCTACCAACAGATCCAAT | 494 | ATATTCATTTGTTAAATTAGGCGCA | 505 |
| 521 | Canine parvovirus | GCTACTATTATGAGACCAGCTGAGG | 506 | AATCCAATCTCCTTCTGGATATCTT | 507 |
| 522 | Canine parvovirus | ATGTATTGCTACCAACAGATCCAAT | 494 | ATTTGTTAAATTAGGCGCAACTTTT | 508 |
| 523 | Canine parvovirus | ACAGGAAGATATCCAGAAGGAGATT | 498 | GTGCATTTACATGAAGTCTTGGTTT | 503 |
| 524 | Canine parvovirus | GAAGATATCCAGAAGGAGATTGGAT | 499 | GTGCATTTACATGAAGTCTTGGTTT | 503 |
| 525 | Canine parvovirus | TAAAAGACGTGGTGTAACTCAAATG | 509 | TGTTGTCTACCAAATGCATATCTTG | 510 |
| 526 | Canine parvovirus | ACTATTATGAGACCAGCTGAGGTTG | 511 | AATCTCCTTCTGGATATCTTCCTGT | 512 |
| 527 | Canine parvovirus | GCTACTATTATGAGACCAGCTGAGG | 506 | AATCTCCTTCTGGATATCTTCCTGT | 512 |
| 528 | Canine parvovirus | GCTACTATTATGAGACCAGCTGAGG | 506 | ATCCAATCTCCTTCTGGATATCTTC | 513 |
| 529 | Canine parvovirus | ACTATTATGAGACCAGCTGAGGTTG | 511 | TGTTGTCTACCAAATGCATATCTTG | 510 |
| 530 | Canine parvovirus | GCTACTATTATGAGACCAGCTGAGG | 506 | TGTTGTCTACCAAATGCATATCTTG | 510 |
| 531 | Canine parvovirus | ACTATTATGAGACCAGCTGAGGTTG | 511 | CATGTTGTCTACCAAATGCATATCT | 514 |
| 532 | Canine parvovirus | GCTACTATTATGAGACCAGCTGAGG | 506 | CATGTTGTCTACCAAATGCATATCT | 514 |
| 533 | Canine parvovirus | CAGGAGAAACACCTGAGAGATTTAC | 515 | CATTTGGATAAACTGGTGGTACATT | 493 |
| 534 | Canine parvovirus | AGGAGAAACACCTGAGAGATTTACA | 516 | CATTTGGATAAACTGGTGGTACATT | 493 |
| 535 | Canine parvovirus | ACAGGAGAAACACCTGAGAGATTTA | 517 | CATTTGGATAAACTGGTGGTACATT | 493 |
| 536 | Canine parvovirus | GGAGTTCAACAAGATAAAAGACGTG | 518 | TGTTGTCTACCAAATGCATATCTTG | 510 |
| 537 | Canine parvovirus | TGAAAATTCTGTGCCAGTACACTTA | 519 | GTATTTCCCATTTGAGTTACACCAC | 520 |
| 538 | Canine parvovirus | ACAGATCCAGATGATGTTCAATTTT | 490 | GTATTTCCCATTTGAGTTACACCAC | 520 |
| 539 | Canine parvovirus | TGAAAATTCTGTGCCAGTACACTTA | 519 | CAACCTCAGCTGGTCTCATAATAGT | 521 |
| 540 | Canine parvovirus | GGCTTACCACCATTTCTAAATTCTT | 522 | CAACCTCAGCTGGTCTCATAATAGT | 521 |
| 541 | Canine parvovirus | ATACATGGCAAACAAATAGAGCATT | 523 | CAACCTCAGCTGGTCTCATAATAGT | 521 |
| 542 | Canine parvovirus | GGCTTACCACCATTTCTAAATTCTT | 522 | TGTTGTCTACCAAATGCATATCTTG | 510 |
| 543 | Canine parvovirus | GGTACAGATCCAGATGATGTTCAAT | 524 | GTATTTCCCATTTGAGTTACACCAC | 520 |
| 544 | Canine parvovirus | TATTGAAAATTCTGTGCCAGTACAC | 525 | GTATTTCCCATTTGAGTTACACCAC | 520 |
| 545 | Canine parvovirus | TATTGAAAATTCTGTGCCAGTACAC | 525 | CAACCTCAGCTGGTCTCATAATAGT | 521 |
| 546 | Canine parvovirus | CAGATCCAGATGATGTTCAATTTTA | 526 | GTATTTCCCATTTGAGTTACACCAC | 520 |
| 547 | Canine parvovirus | CAACAAGATAAAAGACGTGGTGTAA | 527 | TGTTGTCTACCAAATGCATATCTTG | 510 |
| 548 | Canine parvovirus | GGCTTACCACCATTTCTAAATTCTT | 522 | CATGTTGTCTACCAAATGCATATCT | 514 |
| 549 | Canine parvovirus | TATTGAAAATTCTGTGCCAGTACAC | 525 | CACGTCTTTTATCTTGTTGAACTCC | 528 |
| 550 | Canine parvovirus | TGAAAATTCTGTGCCAGTACACTTA | 519 | TACACCACGTCTTTTATCTTGTTGA | 529 |
| 551 | Chlamydophila psittaci | CAGCATCTAATACTATCTTTCTGGAGG | 530 | GGACTACCAGGGTATCTAATCCTGT | 531 |
| 552 | Chlamydophila psittaci | CTGAGAATTTGATCTTGGTTCAGA | 532 | CAACAAGCTGATATCCCATAGACTC | 533 |
| 553 | Chlamydophila psittaci | TTATCGTTAGTTGCCAACACTTAGG | 534 | ATTACTAGCAATTCCGACTTCATGT | 535 |
| 554 | Chlamydophila psittaci | CCTTATCGTTAGTTGCCAACACTTA | 536 | ATTACTAGCAATTCCGACTTCATGT | 535 |
| 555 | Chlamydophila psittaci | CCAGCATCTAATACTATCTTTCTGGAG | 537 | GGACTACCAGGGTATCTAATCCTGT | 531 |
| 556 | Chlamydophila psittaci | TCTTTCTAGAGGGTAGATGGAGAAAA | 538 | GGACTACCAGGGTATCTAATCCTGT | 531 |
| 557 | Chlamydophila psittaci | TCTTTCTAGAGGGTAGATGGAGAAAA | 538 | ATAGGGTTGAGACTATCCACATCAA | 539 |
| 558 | Chlamydophila psittaci | TCTTTCTAGAGGGTAGATGGAGAAAA | 538 | TTGAGACTATCCACATCAAGTATGC | 540 |
| 559 | Chlamydophila psittaci | TCTTTCTAGAGGGTAGATGGAGAAAA | 538 | CAAGGACTACCAGGGTATCTAATCC | 541 |
| 560 | Chlamydophila psittaci | CTTTCTAGAGGGTAGATGGAGAAAAG | 542 | GGACTACCAGGGTATCTAATCCTGT | 531 |
| 561 | Chlamydophila psittaci | CTTTCTAGAGGGTAGATGGAGAAAAG | 542 | ATAGGGTTGAGACTATCCACATCAA | 539 |
| 562 | Chlamydophila psittaci | TCTTTCTAGAGGGTAGATGGAGAAAA | 538 | GCAAGGACTACCAGGGTATCTAATC | 543 |
| 563 | Chlamydophila psittaci | TCTTTCTAGAGGGTAGATGGAGAAAA | 538 | GATAGGGTTGAGACTATCCACATCA | 544 |
| 564 | Chlamydophila psittaci | ATCTTTCTAGAGGGTAGATGGAGAAA | 545 | GGACTACCAGGGTATCTAATCCTGT | 531 |
| 565 | Chlamydophila psittaci | TCTTTCTAGAGGGTAGATGGAGAAAA | 538 | GGGTTGAGACTATCCACATCAAGTA | 546 |
| 566 | Chlamydophila psittaci | CAAGCCAGCATCTAATACTATCTTTC | 547 | GGACTACCAGGGTATCTAATCCTGT | 531 |
| 567 | Chlamydophila psittaci | AAAGTTAGATGTTAAATCTTGGGGC | 548 | GGTTGAGACTATCCACATCAAGTATG | 549 |
| 568 | Chlamydophila psittaci | AAGTTAGATGTTAAATCTTGGGGCT | 550 | GGTTGAGACTATCCACATCAAGTATG | 549 |
| 569 | Chlamydophila psittaci | AGTTAGATGTTAAATCTTGGGGCTC | 551 | GGTTGAGACTATCCACATCAAGTATG | 549 |
| 570 | Chlamydophila psittaci | AAAGTTAGATGTTAAATCTTGGGGC | 548 | GTTGAGACTATCCACATCAAGTATGC | 552 |
| 571 | Chlamydophila psittaci | AAGTTAGATGTTAAATCTTGGGGCT | 550 | GTTGAGACTATCCACATCAAGTATGC | 552 |
| 572 | Chlamydophila psittaci | AGTTAGATGTTAAATCTTGGGGCTC | 551 | GTTGAGACTATCCACATCAAGTATGC | 552 |
| 573 | Chlamydophila psittaci | AAAGTTAGATGTTAAATCTTGGGGC | 548 | GACTACCAGGGTATCTAATCCTGTTT | 553 |
| 574 | Chlamydophila psittaci | AAGTTAGATGTTAAATCTTGGGGCT | 550 | GACTACCAGGGTATCTAATCCTGTTT | 553 |
| 575 | Chlamydophila psittaci | AAAGTTAGATGTTAAATCTTGGGGCT | 554 | GGACTACCAGGGTATCTAATCCTGT | 531 |
| 576 | Chlamydophila psittaci | AGTTAGATGTTAAATCTTGGGGCTC | 551 | GACTACCAGGGTATCTAATCCTGTTT | 553 |
| 577 | Chlamydophila psittaci | AAAGTTAGATGTTAAATCTTGGGGC | 548 | AGGACTACCAGGGTATCTAATCCTGT | 555 |
| 578 | Chlamydophila psittaci | AAGTTAGATGTTAAATCTTGGGGCT | 550 | AGGACTACCAGGGTATCTAATCCTGT | 555 |
| 579 | Chlamydophila psittaci | AGTTAGATGTTAAATCTTGGGGCTC | 551 | AGGACTACCAGGGTATCTAATCCTGT | 555 |
| 580 | Chlamydophila psittaci | AAAGTTAGATGTTAAATCTTGGGGC | 548 | CTTAGCGTCAGGTGTAAATTAGAAAA | 556 |
| 581 | Chlamydophila psittaci | AAAGTTAGATGTTAAATCTTGGGGC | 548 | TTAGCGTCAGGTGTAAATTAGAAAAG | 557 |
| 582 | Chlamydophila psittaci | AAGTTAGATGTTAAATCTTGGGGCT | 550 | CTTAGCGTCAGGTGTAAATTAGAAAA | 556 |
| 583 | Chlamydophila psittaci | AAGTTAGATGTTAAATCTTGGGGCT | 550 | TTAGCGTCAGGTGTAAATTAGAAAAG | 557 |
| 584 | Chlamydophila psittaci | AGTTAGATGTTAAATCTTGGGGCTC | 551 | CTTAGCGTCAGGTGTAAATTAGAAAA | 556 |
| 585 | Chlamydophila psittaci | AGTTAGATGTTAAATCTTGGGGCTC | 551 | TTAGCGTCAGGTGTAAATTAGAAAAG | 557 |
| 586 | Chlamydophila psittaci | AAAGTTAGATGTTAAATCTTGGGGCT | 554 | CCTTAGCGTCAGGTGTAAATTAGAA | 558 |
| 587 | Chlamydophila psittaci | AGGAAAGTTAGATGTTAAATCTTGGG | 559 | GGACTACCAGGGTATCTAATCCTGT | 531 |
| 588 | Chlamydophila psittaci | AGGAAAGTTAGATGTTAAATCTTGGG | 559 | ATAGGGTTGAGACTATCCACATCAA | 539 |
| 589 | Chlamydophila psittaci | AGGAAAGTTAGATGTTAAATCTTGGG | 559 | CAAGGACTACCAGGGTATCTAATCC | 541 |
| 590 | Chlamydophila psittaci | AGGAAAGTTAGATGTTAAATCTTGGG | 559 | GCAAGGACTACCAGGGTATCTAATC | 543 |
| 591 | Chlamydophila psittaci | TTCTAGAGGGTAGATGGAGAAAAGG | 560 | GTTGAGACTATCCACATCAAGTATGC | 552 |
| 592 | Chlamydophila psittaci | TTCTAGAGGGTAGATGGAGAAAAGG | 560 | GGTTGAGACTATCCACATCAAGTATG | 549 |
| 593 | Chlamydophila psittaci | AGGAAAGTTAGATGTTAAATCTTGGG | 559 | CCTTAGCGTCAGGTGTAAATTAGAA | 558 |
| 594 | Chlamydophila psittaci | AATGACTTCGGTTGTTATTTAGTGG | 561 | AACAAGCTGATATCCCATAGACTCTC | 562 |
| 595 | Chlamydophila psittaci | AACATATTAAAGAAGGGGATCTTCG | 563 | ATATTAGCCAATCTCTCTATTCCCA | 564 |
| 596 | Chlamydophila psittaci | AACATATTAAAGAAGGGGATCTTCG | 563 | TATTAGCCAATCTCTCTTATTCCCAA | 565 |
| 597 | Chlamydophila psittaci | TAATGACTTCGGTTGTTATTTAGTGG | 566 | CAACAAGCTGATATCCCATAGACTC | 533 |
| 598 | Chlamydophila psittaci | GGAAAGTTAGGTGTTAAATCTTGGG | 567 | GGTTGAGACTATCCACATCAAGTATG | 549 |
| 599 | Chlamydophila psittaci | GAAAGTTAGGTGTTAAATCTTGGGG | 568 | GGTTGAGACTATCCACATCAAGTATG | 549 |
| 600 | Chlamydophila psittaci | GCATCTAAAACATATTAAAGAAGGGG | 569 | TATTAGCCAATCTCTCTTATTCCCA | 570 |
| 601 | Clostridium difficile | TGAAGAAAGAGAAAAGATAGCTGATG | 571 | AACATTAGTTCCATAACTTGCTCCA | 572 |
| 602 | Clostridium difficile | ATCAAATGGAGCATGGTTAGTAAAA | 573 | ACCAGCATATTCAAGTTGGTACATT | 574 |
| 603 | Clostridium difficile | AATCAAATGGAGCATGGTTAGTAAA | 575 | ACCAGCATATTCAAGTTGGTACATT | 574 |
| 604 | Clostridium difficile | CAAATGGAGCATGGTTAGTAAAAAG | 576 | ACCAGCATATTCAAGTTGGTACATT | 574 |
| 605 | Clostridium difficile | ATGAAAGGATTCTTCTGGATTTCTT | 577 | AAAATCAAATACATTTGCTGCTGTT | 578 |
| 606 | Clostridium difficile | AGAATCAAATGGAGCATGGTTAGTA | 579 | AAAATCAAATACATTTGCTGCTGTT | 578 |
| 607 | Clostridium difficile | GACATCAGGTTATGCACATTATTCA | 580 | AAGAAATCCAGAAGAATCCTTTCAT | 581 |
| 608 | Clostridium difficile | TGCTATAATGCAATATGGTTCAGTG | 582 | AAGAAATCCAGAAGAATCCTTTCAT | 581 |
| 609 | Clostridium difficile | ATCAAATGGAGCATGGTTAGTAAAA | 573 | AAAATCAAATACATTTGCTGCTGTT | 578 |
| 610 | Clostridium difficile | AATCAAATGGAGCATGGTTAGTAAA | 575 | AAAATCAAATACATTTGCTGCTGTT | 578 |
| 611 | Clostridium difficile | TCAGGTTATGCACATTATTCAACAT | 583 | AAGAAATCCAGAAGAATCCTTTCAT | 581 |
| 612 | Clostridium difficile | ATCAGGTTATGCACATTATTCAACA | 584 | AAGAAATCCAGAAGAATCCTTTCAT | 581 |
| 613 | Clostridium difficile | CATCAGGTTATGCACATTATTCAAC | 585 | AAGAAATCCAGAAGAATCCTTTCAT | 581 |
| 614 | Clostridium difficile | ACATCAGGTTATGCACATTATTCAA | 586 | AAGAAATCCAGAAGAATCCTTTCAT | 581 |
| 615 | Clostridium difficile | TGACATCAGGTTATGCACATTATTC | 587 | TAAGAAATCCAGAAGAATCCTTTCA | 588 |
| 616 | Clostridium difficile | GACATCAGGTTATGCACATTATTCA | 580 | TAAGAAATCCAGAAGAATCCTTTCA | 588 |
| 617 | Clostridium difficile | TAATGCAATATGGTTCAGTGACATC | 589 | TAAGAAATCCAGAAGAATCCTTTCA | 588 |
| 618 | Clostridium difficile | CAAATGGAGCATGGTTAGTAAAAAG | 576 | AAAATCAAATACATTTGCTGCTGTT | 578 |
| 619 | Clostridium difficile | TAGCGATAGTAGGATGGGATGATAA | 590 | ACCAGCATATTCAAGTTGGTACATT | 574 |
| 620 | Clostridium difficile | AGAATCAAATGGAGCATGGTTAGTA | 579 | GACATTATCTTGCTAAGACCAGCAT | 591 |
| 621 | Clostridium difficile | ATGAAAGGATTCTTCTGGATTTCTT | 577 | TTTGCTGCTGTTACTTTATTTGACA | 592 |
| 622 | Clostridium difficile | ATGAAAGGATTCTTCTGGATTTCTT | 577 | CATTTGCTGCTGTTACTTTATTTGA | 593 |
| 623 | Clostridium difficile | TGACATCAGGTTATGCACATTATTC | 587 | TACTAACCATGCTCCATTTGATTCT | 594 |
| 624 | Clostridium difficile | GACATCAGGTTATGCACATTATTCA | 580 | TACTAACCATGCTCCATTTGATTCT | 594 |
| 625 | Clostridium difficile | ATCAGGTTATGCACATTATTCAACA | 584 | TACTAACCATGCTCCATTTGATTCT | 594 |
| 626 | Clostridium difficile | TCAGGTTATGCACATTATTCAACAT | 583 | TACTAACCATGCTCCATTTGATTCT | 594 |
| 627 | Clostridium difficile | CATCAGGTTATGCACATTATTCAAC | 585 | TACTAACCATGCTCCATTTGATTCT | 594 |
| 628 | Clostridium difficile | ACATCAGGTTATGCACATTATTCAA | 586 | TACTAACCATGCTCCATTTGATTCT | 594 |
| 629 | Clostridium difficile | TGAAAGGATTCTTCTGGATTTCTTA | 595 | TTTGCTGCTGTTACTTTATTTGACA | 592 |
| 630 | Clostridium difficile | TGAAAGGATTCTTCTGGATTTCTTA | 595 | CATTTGCTGCTGTTACTTTATTTGA | 593 |
| 631 | Clostridium difficile | ATCAAATGGAGCATGGTTAGTAAAA | 573 | GACATTATCTTGCTAAGACCAGCAT | 591 |
| 632 | Clostridium difficile | AATCAAATGGAGCATGGTTAGTAAA | 575 | GACATTATCTTGCTAAGACCAGCAT | 591 |
| 633 | Clostridium difficile | TGACATCAGGTTATGCACATTATTC | 587 | TTTTACTAACCATGCTCCATTTGAT | 596 |
| 634 | Clostridium difficile | GACATCAGGTTATGCACATTATTCA | 580 | TTTACTAACCATGCTCCATTTGATT | 597 |
| 635 | Clostridium difficile | TGACATCAGGTTATGCACATTATTC | 587 | TTTACTAACCATGCTCCATTTGATT | 597 |
| 636 | Clostridium difficile | GACATCAGGTTATGCACATTATTCA | 580 | TTTTACTAACCATGCTCCATTTGAT | 596 |
| 637 | Clostridium difficile | GTAGCGATAGTAGGATGGGATGATA | 598 | ACCAGCATATTCAAGTTGGTACATT | 574 |
| 638 | Clostridium difficile | CAAATGGAGCATGGTTAGTAAAAAG | 576 | GACATTATCTTGCTAAGACCAGCAT | 591 |
| 639 | Clostridium difficile | TGACATCAGGTTATGCACATTATTC | 587 | CTTTTTACTAACCATGCTCCATTTG | 599 |
| 640 | Clostridium difficile | GACATCAGGTTATGCACATTATTCA | 580 | CTTTTTACTAACCATGCTCCATTTG | 599 |
| 641 | Clostridium difficile | ATCAGGTTATGCACATTATTCAACA | 584 | TTTTACTAACCATGCTCCATTTGAT | 596 |
| 642 | Clostridium difficile | ATCAGGTTATGCACATTATTCAACA | 584 | TTTACTAACCATGCTCCATTTGATT | 597 |
| 643 | Clostridium difficile | TCAGGTTATGCACATTATTCAACAT | 583 | TTTTACTAACCATGCTCCATTTGAT | 596 |
| 644 | Clostridium difficile | CATCAGGTTATGCACATTATTCAAC | 585 | TTTACTAACCATGCTCCATTTGATT | 597 |
| 645 | Clostridium difficile | CATCAGGTTATGCACATTATTCAAC | 585 | TTTTACTAACCATGCTCCATTTGAT | 596 |
| 646 | Clostridium difficile | ACATCAGGTTATGCACATTATTCAA | 586 | TTTTACTAACCATGCTCCATTTGAT | 596 |
| 647 | Clostridium difficile | AGAATCAAATGGAGCATGGTTAGTA | 579 | TTTGCTGCTGTTACTTTATTTGACA | 592 |
| 648 | Clostridium difficile | AGAATCAAATGGAGCATGGTTAGTA | 579 | CATTTGCTGCTGTTACTTTATTTGA | 593 |
| 649 | Clostridium difficile | CATCAGGTTATGCACATTATTCAAC | 585 | CTTTTTACTAACCATGCTCCATTTG | 599 |
| 650 | Clostridium difficile | TCAGGTTATGCACATTATTCAACAT | 583 | CTTTTTACTAACCATGCTCCATTTG | 599 |
| 651 | Cryptosporidium spp | AAGAGGTGTACCACAAATTGAAGTC | 600 | AATCTTAAGTCTGTTTTGCTCATCC | 601 |
| 652 | Cryptosporidium spp | CTGGTTTTGTTGAAGAAGGAAATAG | 602 | CATCTCCTGAGTCTGTATATCGTGG | 603 |
| 653 | Cryptosporidium spp | CATCATCATCATCATCATCAACATC | 604 | CCACATTCTTCCTTTGGAGTAGTAGT | 605 |
| 654 | Cryptosporidium spp | CATCATCATCATCATCATCATCAAC | 606 | CCACATTCTTCCTTTGGAGTAGTAGT | 605 |
| 655 | Cryptosporidium spp | ATCATCATCATCATCAACATCGAC | 607 | CCACATTCTTCCTTTGGAGTAGTAG | 608 |
| 656 | Cryptosporidium spp | CATCATCATCATCATCATCAACATC | 604 | CCACATTCTTCCTTTGGAGTAGTAG | 608 |
| 657 | Cryptosporidium spp | CATCATCATCATCATCATCATCAAC | 606 | CCACATTCTTCCTTTGGAGTAGTAG | 608 |
| 658 | Cryptosporidium spp | CTGAAGAACAAGACGGTGGTAAG | 609 | CCACATTCTTCCTTTGGAGTAGTAG | 608 |
| 659 | Cryptosporidium spp | CCAAAGAAGGAAAGAACTGTAGAGG | 610 | CCACATTCTTCCTTTGGAGTAGTAGT | 605 |
| 660 | Cryptosporidium spp | TACCATGAATAAGGCGCTATAAAATA | 611 | AAGACATAATTTATCTTCAATGCCG | 612 |
| 661 | Cryptosporidium spp | TACCATGGATAAGGCGCTATAAAATA | 613 | AAGACATAATTTATCTTCAATGCCG | 612 |
| 662 | Cryptosporidium spp | AGAAAAAGAATGCAGTTTACTATCCAG | 614 | AAGACATAATTTATCTTCAATGCCG | 612 |
| 663 | Cryptosporidium spp | GAAAAAGAATGCAGTTTACTATCCAGT | 615 | AAGACATAATTTATCTTCAATGCCG | 612 |
| 664 | Cryptosporidium spp | GAGAAAAAGAATGCAGTTTACTATCCA | 616 | AAGACATAATTTATCTTCAATGCCG | 612 |
| 665 | Cryptosporidium spp | TACCATGAATAAGGCGCTATAAAAT | 617 | AAGACATAATTTATCTTCAATGCCG | 612 |
| 666 | Cryptosporidium spp | AGCTCCAAAGAAAGAAAGAACTGTAG | 618 | CACTTTCAGATGGAGTAACTTGAGAA | 619 |
| 667 | Cryptosporidium spp | CAAAGAAGGAAAGAACTGTAGAGGG | 620 | CCACATTCTTCCTTTGGAGTAGTAGT | 605 |
| 668 | Cryptosporidium spp | TTTTGAATCAATAGAGAAAAAGAATGC | 621 | AAGACATAATTTATCTTCAATGCCG | 612 |
| 669 | Cryptosporidium spp | AAAGAAAGAAAGAGCTGTAGAGGGC | 622 | CCACATTCTTCCTTTGGAGTAGTAG | 608 |
| 670 | Cryptosporidium spp | TCCAAAGAAAGAAAGAACTGTAGAGG | 623 | CACTTTCAGATGGAGTAACTTGAGAA | 619 |
| 671 | Cryptosporidium spp | TACCATGGATAAGGCGCTATAAAAT | 624 | AAGACATAATTTATCTTCAATGCCG | 612 |
| 672 | Cryptosporidium spp | TGAATCAATAGAGAAAAAGAATGCAG | 625 | AAGACATAATTTATCTTCAATGCCGT | 626 |
| 673 | Cryptosporidium spp | TACCATGAATAAGGCGCTATAAAAT | 617 | CAAGACGTAATTTATCTTCAATGCTG | 627 |
| 674 | Cryptosporidium spp | ATCATCATCATCATCATCGTCATC | 628 | CCACATTCTTCCTTTGGAGTAGTAG | 608 |
| 675 | Cryptosporidium spp | ATCATCATCATCATCATCATCGTC | 629 | CCACATTCTTCCTTTGGAGTAGTAG | 608 |
| 676 | Cryptosporidium spp | AAAAAGAATGCAGTTTACTATCCAGTG | 630 | AAGACATAATTTATCTTCAATGCCG | 612 |
| 677 | Cryptosporidium spp | CCAAAGAAGGAAAGAACTGTAGAGG | 610 | CCACATTCTTCCTTTGGAGTAGTAG | 608 |
| 678 | Cryptosporidium spp | ATCATCATCATCATCGTCATCATC | 631 | CCACATTCTTCCTTTGGAGTAGTAG | 608 |
| 679 | Cryptosporidium spp | ATCATCATCGTCATCATCAACATC | 632 | CCACATTCTTCCTTTGGAGTAGTAG | 608 |
| 680 | Cryptosporidium spp | ATCATCATCATCGTCATCATCAAC | 633 | CCACATTCTTCCTTTGGAGTAGTAG | 608 |
| 681 | Cryptosporidium spp | AAGAAAGCAGTCAAGGTTCTGAAG | 634 | CCACATTCTTCCTTTGGAGTAGTAG | 608 |
| 682 | Cryptosporidium spp | GAAAAAGAATGCAGTTTACTATCCAG | 635 | AAGACATAATTTATCTTCAATGCCG | 612 |
| 683 | Cryptosporidium spp | TACCATGGATAAGGCGCTATAAAA | 636 | AAGACATAATTTATCTTCAATGCCG | 612 |
| 684 | Cryptosporidium spp | AGAAAAAGAATGCAGTTTACTATCCA | 637 | AAGACATAATTTATCTTCAATGCCG | 612 |
| 685 | Cryptosporidium spp | AAGAAAGAAAGAGCTGTAGAGGGC | 638 | CCACATTCTTCCTTTGGAGTAGTAG | 608 |
| 686 | Cryptosporidium spp | CAGCTCCAAAGAAAGAAAGAACTGT | 639 | CACTTTCAGATGGAGTAACTTGAGAA | 619 |
| 687 | Cryptosporidium spp | GCTCCAAAGAAAGAAAGAACTGTAG | 640 | CTTTCAGATGGAGTAACTTGAGAACC | 641 |
| 688 | Cryptosporidium spp | TTTGAATCAATAGAGAAAAAGAATGC | 642 | AAGACATAATTTATCTTCAATGCCG | 612 |
| 689 | Cryptosporidium spp | GCTCCAAAGAAAGAAAGAACTGTAG | 640 | CACTTTCAGATGGAGTAACTTGAGAA | 619 |
| 690 | Cryptosporidium spp | CAGCTCCAAAGAAAGAAAGAACTGTA | 643 | TTTCAGATGGAGTAACTTGAGAACC | 644 |
| 691 | Cryptosporidium spp | CAGCTCCAAAGAAAGAAAGAACTGTA | 643 | CAGATGGAGTAACTTGAGAACCACT | 645 |
| 692 | Cryptosporidium spp | AGCTCCAAAGAAAGAAAGAACTGTAG | 618 | TTTCAGATGGAGTAACTTGAGAACC | 644 |
| 693 | Cryptosporidium spp | AGCTCCAAAGAAAGAAAGAACTGTAG | 618 | CAGATGGAGTAACTTGAGAACCACT | 645 |
| 694 | Cryptosporidium spp | AGCTCCAAAGAAAGAAAGAACTGTAG | 618 | GTAGTAGTTGCTGTGGACTCGGTAG | 646 |
| 695 | Cryptosporidium spp | AGCTCCAAAGAAAGAAAGAACTGTAG | 618 | AGTAGTAGTTGCTGTGGACTCGGTA | 647 |
| 696 | Cryptosporidium spp | AGCTCCAAAGAAAGAAAGAACTGTAG | 618 | GCACTTTCAGATGGAGTAACTTGAG | 648 |
| 697 | Cryptosporidium spp | AGCTCCAAAGAAAGAAAGAACTGTAG | 618 | AGATGGAGTAACTTGAGAACCACTC | 649 |
| 698 | Cryptosporidium spp | TACCATGAATAAGGCGCTATAAAAT | 617 | TCAAGACATAATTTATCTTCAATGCC | 650 |
| 699 | Cryptosporidium spp | GCTCCAAAGAAAGAAAGAACTGTAGA | 651 | TTTCAGATGGAGTAACTTGAGAACC | 644 |
| 700 | Cryptosporidium spp | GCTCCAAAGAAAGAAAGAACTGTAGA | 651 | CAGATGGAGTAACTTGAGAACCACT | 645 |
| 701 | Dirofilaria immitis | ATTATTATTAATCAAGCCATTTTTCGAT | 652 | TAGGACTTTCCAAAATTTCATTTCA | 653 |
| 702 | Dirofilaria immitis | TATTATTAATCAAGCCATTTTTCGATG | 654 | TAGGACTTTCCAAAATTTCATTTCA | 653 |
| 703 | Dirofilaria immitis | TTATTATTAATCAAGCCATTTTTCGAT | 655 | TAGGACTTTCCAAAATTTCATTTCA | 653 |
| 704 | Dirofilaria immitis | ATTATTAATCAAGCCATTTTTCGATG | 656 | TAGGACTTTCCAAAATTTCATTTCA | 653 |
| 705 | Dirofilaria immitis | TATTATTAATCAAGCCATTTTTCGAT | 657 | TAGGACTTTCCAAAATTTCATTTCA | 653 |
| 706 | Dirofilaria immitis | TTATTAATCAAGCCATTTTTCGATG | 658 | TAGGACTTTCCAAAATTTCATTTCAA | 659 |
| 707 | Dirofilaria immitis | ATTATTATTAATCAAGCCATTTTTCG | 660 | TAGGACTTTCCAAAATTTCATTTCA | 653 |
| 708 | Dirofilaria immitis | TTATTAATCAAGCCATTTTTCGATG | 658 | TAGGACTTTCCAAAATTTCATTTCA | 653 |
| 709 | Dirofilaria immitis | TTATTAATCAAGCCATTTTTCGATG | 658 | TAGGACTTTCCAAAATTTCATTTCAC | 661 |
| 710 | Dirofilaria immitis | TTATTAATCAAGCCATTTTTCGATG | 658 | TAGGACTTTCCAAAATTTCATTTCAG | 662 |
| 711 | Dirofilaria immitis | ATTATTAATCAAGCCATTTTTCGAT | 663 | TAGGACTTTCCAAAATTTCATTTCA | 653 |
| 712 | Dirofilaria immitis | ATTATTAATCAAGCCATTTTTCGAT | 663 | TAGGACTTTCCAAAATTTCATTTCAC | 661 |
| 713 | Dirofilaria immitis | TATTAATCAAGCCATTTTTCGATG | 664 | TAGGACTTTCCAAAATTTCATTTCA | 653 |
| 714 | Dirofilaria immitis | AATCCCAGGTGTTGTAGACATTTTT | 665 | TAGGACTTTCCAAAATTTCATTTCA | 653 |
| 715 | Dirofilaria immitis | AATCCCAGGTGTTGTAGACATTTTT | 665 | TAGGACTTTCCAAAATTTCATTTCAG | 662 |
| 716 | Dirofilaria immitis | TTATTAATCAAGCCATTTTTCGAT | 666 | TAGGACTTTCCAAAATTTCATTTCA | 653 |
| 717 | Dirofilaria immitis | ATCCCAGGTGTTGTAGACATTTTT | 667 | TAGGACTTTCCAAAATTTCATTTCA | 653 |
| 718 | Dirofilaria immitis | GAATCCCAGGTGTTGTAGACATTT | 668 | TAGGACTTTCCAAAATTTCATTTCA | 653 |
| 719 | Dirofilaria immitis | ATCCCAGGTGTTGTAGACATTTTT | 667 | TAGGACTTTCCAAAATTTCATTTCAG | 662 |
| 720 | Dirofilaria immitis | AATCCCAGGTGTTGTAGACATTTT | 669 | TAGGACTTTCCAAAATTTCATTTCAG | 662 |
| 721 | Dirofilaria immitis | GTCCGATCTGTCAAGTTAAGCAAC | 670 | TAGGACTTTCCAAAATTTCATTTCA | 653 |
| 722 | Dirofilaria immitis | TCTCGTCCGATCTGTCAAGTTAAG | 671 | TAGGACTTTCCAAAATTTCATTTCA | 653 |
| 723 | Dirofilaria immitis | TCCCAGGTGTTGTAGACATTTTT | 672 | TAGGACTTTCCAAAATTTCATTTCA | 653 |
| 724 | Dirofilaria immitis | GAATCCCAGGTGTTGTAGACATT | 673 | TAGGACTTTCCAAAATTTCATTTCA | 653 |
| 725 | Dirofilaria immitis | AATCCCAGGTGTTGTAGACATTT | 674 | TAGGACTTTCCAAAATTTCATTTCA | 653 |
| 726 | Dirofilaria immitis | ATCTCGTCCGATCTGTCAAGTTA | 675 | TAGGACTTTCCAAAATTTCATTTCA | 653 |
| 727 | Dirofilaria immitis | CTCGTCCGATCTGTCAAGTTAAG | 676 | TAGGACTTTCCAAAATTTCATTTCA | 653 |
| 728 | Dirofilaria immitis | CTGGTTAGTACTTGGATCGGAGA | 677 | TAGGACTTTCCAAAATTTCATTTCA | 653 |
| 729 | Dirofilaria immitis | TCCGATCTGTCAAGTTAAGCAAC | 678 | TAGGACTTTCCAAAATTTCATTTCA | 653 |
| 730 | Dirofilaria immitis | GTCCGATCTGTCAAGTTAAGCAA | 679 | TAGGACTTTCCAAAATTTCATTTCA | 653 |
| 731 | Dirofilaria immitis | TTATTATTAATCAAGCCATTTTTCGAT | 655 | AGGACTTTCCAAAATTTCATTTCAC | 680 |
| 732 | Dirofilaria immitis | ATTATTAATCAAGCCATTTTTCGATG | 656 | GGACTTTCCAAAATTTCATTTCAAG | 681 |
| 733 | Dirofilaria immitis | ATTATTAATCAAGCCATTTTTCGATG | 656 | AGGACTTTCCAAAATTTCATTTCAA | 682 |
| 734 | Dirofilaria immitis | ATTATTAATCAAGCCATTTTTCGATG | 656 | AGGACTTTCCAAAATTTCATTTCAC | 680 |
| 735 | Dirofilaria immitis | ATTATTAATCAAGCCATTTTTCGATG | 656 | AGGACTTTCCAAAATTTCATTTCAG | 683 |
| 736 | Dirofilaria immitis | ATTATTAATCAAGCCATTTTTCGATG | 656 | AGGACTTTCCAAAATTTCATTTCA | 684 |
| 737 | Dirofilaria immitis | AATCCCAGGTGTTGTAGACATTTTT | 665 | CAGATATAATTCTGGGCTGAAACATT | 685 |
| 738 | Dirofilaria immitis | AATCCCAGGTGTTGTAGACATTTTT | 665 | GCTGAAACATTCAATTACCTCAAACT | 686 |
| 739 | Dirofilaria immitis | AATCCCAGGTGTTGTAGACATTTTT | 665 | GAAACATTCAATTACCTCAAACTTGG | 687 |
| 740 | Dirofilaria immitis | AATCCCAGGTGTTGTAGACATTTTT | 665 | CTGAAACATTCAATTACCTCAAACTT | 688 |
| 741 | Dirofilaria immitis | TATTATTAATCAAGCCATTTTTCGAT | 657 | AGGACTTTCCAAAATTTCATTTCAC | 680 |
| 742 | Dirofilaria immitis | TTATTAATCAAGCCATTTTTCGATG | 658 | AGGACTTTCCAAAATTTCATTTCAA | 682 |
| 743 | Dirofilaria immitis | AATCCCAGGTGTTGTAGACATTTTT | 665 | ATTTCAAGCCACAGCAGATATAATTC | 689 |
| 744 | Dirofilaria immitis | ATTATTAATCAAGCCATTTTTCGATG | 656 | GGACTTTCCAAAATTTCATTTCAA | 690 |
| 745 | Dirofilaria immitis | ATTATTAATCAAGCCATTTTTCGAT | 663 | GGACTTTCCAAAATTTCATTTCAAG | 681 |
| 746 | Dirofilaria immitis | ATTATTAATCAAGCCATTTTTCGAT | 663 | AGGACTTTCCAAAATTTCATTTCAA | 682 |
| 747 | Dirofilaria immitis | TTATTAATCAAGCCATTTTTCGATG | 658 | AGGACTTTCCAAAATTTCATTTCAC | 680 |
| 748 | Dirofilaria immitis | TTATTAATCAAGCCATTTTTCGATG | 658 | AGGACTTTCCAAAATTTCATTTCAG | 683 |
| 749 | Dirofilaria immitis | TTATTAATCAAGCCATTTTTCGATG | 658 | AGGACTTTCCAAAATTTCATTTCA | 684 |
| 750 | Dirofilaria immitis | AATCCCAGGTGTTGTAGACATTTTT | 665 | AGCAGATATAATTCTGGGCTGAAAC | 691 |
| 751 | Echinococcus granulosus | CACATTTGTCACCTCCCTTTTAGTA | 692 | GCATGACTTACTTTGAATCATCATCT | 693 |
| 752 | Echinococcus granulosus | TCTTCTCTCCATACCTCACTTTGAC | 694 | CTTGACATATTTCTTCAACACTTCGT | 695 |
| 753 | Echinococcus granulosus | TCTGTGTTATGATTTTTAGCTGCTTC | 696 | ATGGGAATAATAATCAACTTCACCA | 697 |
| 754 | Echinococcus granulosus | CTTTCTGTGTTATGATTTTTAGCTGC | 698 | ATGGGAATAATAATCAACTTCACCA | 697 |
| 755 | Echinococcus granulosus | GCTTTCTGTGTTATGATTTTTAGCTG | 699 | ATGGGAATAATAATCAACTTCACCA | 697 |
| 756 | Echinococcus granulosus | TCTAGGTATTCCTTGTTGTGTACTGG | 700 | ATGGGAATAATAATCAACTTCACCA | 697 |
| 757 | Echinococcus granulosus | TTGCCGATTTGTTGAAGTTAGTAAT | 701 | GAAGCAGCTAAAAATCATAACACAGA | 702 |
| 758 | Echinococcus granulosus | TTGCCGATTTGTTGAAGTTAGTAAT | 701 | GCAGCTAAAAATCATAACACAGAAAG | 703 |
| 759 | Echinococcus granulosus | TTCTGTGTTATGATTTTTAGCTGCTT | 704 | ATGGGAATAATAATCAACTTCACCA | 697 |
| 760 | Echinococcus granulosus | TTGCCGATTTGTTGAAGTTAGTAAT | 701 | CAGCTAAAAATCATAACACAGAAAGC | 705 |
| 761 | Echinococcus granulosus | CTGTGTTATGATTTTTAGCTGCTTC | 706 | ATGGGAATAATAATCAACTTCACCA | 697 |
| 762 | Echinococcus granulosus | TTGCCGATTTGTTGAAGTTAGTAAT | 701 | CTATTGTAACTACCCCAACCAGTACA | 707 |
| 763 | Echinococcus granulosus | CTTTCAGAGTCGTAGGTATGTTGGT | 708 | CTATTGTAACTACCCCAACCAGTACA | 707 |
| 764 | Echinococcus granulosus | TTCAGAGTCGTAGGTATGTTGGTTT | 709 | CTATTGTAACTACCCCAACCAGTACA | 707 |
| 765 | Echinococcus granulosus | TTGCCGATTTGTTGAAGTTAGTAAT | 701 | AGCAGCTAAAAATCATAACACAGAAA | 710 |
| 766 | Echinococcus granulosus | TTGCCGATTTGTTGAAGTTAGTAAT | 701 | AAGCAGCTAAAAATCATAACACAGAA | 711 |
| 767 | Echinococcus granulosus | ATTTGTGGAGACAATTGCATAATGA | 712 | CATCTTTACTAAAAGGGAGGTGACA | 713 |
| 768 | Echinococcus granulosus | ATTTGTGGAGACAATTGCATAATGA | 712 | TTTACTAAAAGGGAGGTGACAAATG | 714 |
| 769 | Echinococcus granulosus | TTTCTGTGTTATGATTTTTAGCTGC | 715 | ATGGGAATAATAATCAACTTCACCA | 697 |
| 770 | Echinococcus granulosus | TGTGTTATGATTTTTAGCTGCTTCT | 716 | ATGGGAATAATAATCAACTTCACCA | 697 |
| 771 | Echinococcus granulosus | TCTGTGTTATGATTTTTAGCTGCTT | 717 | ATGGGAATAATAATCAACTTCACCA | 697 |
| 772 | Echinococcus granulosus | AGGTATTCCTTGTTGTGTACTGGTT | 718 | ATGGGAATAATAATCAACTTCACCA | 697 |
| 773 | Echinococcus granulosus | AGGTTTGCTGATTTGTTGAAGTTAG | 719 | GAAGCAGCTAAAAATCATAACACAGA | 702 |
| 774 | Echinococcus granulosus | TCTGTGTTATGATTTTTAGCTGCTTC | 696 | ATAACCACACACATAAAACAAGCCT | 720 |
| 775 | Echinococcus granulosus | AGGTTTGCTGATTTGTTGAAGTTAG | 719 | GCAGCTAAAAATCATAACACAGAAAG | 703 |
| 776 | Echinococcus granulosus | CTTTCTGTGTTATGATTTTTAGCTGC | 698 | ATAACCACACACATAAAACAAGCCT | 720 |
| 777 | Echinococcus granulosus | TCTAGGTATTCCTTGTTGTGTACTGG | 700 | ATTAGTCTCACACAACACACACACC | 721 |
| 778 | Echinococcus granulosus | CTTTCAGAGTCGTAGGTATGTTGGT | 708 | AATTATACCCACAACAGCATAAAGC | 722 |
| 779 | Echinococcus granulosus | TTTCAGAGTCGTAGGTATGTTGGTT | 723 | AATTATACCCACAACAGCATAAAGC | 722 |
| 780 | Echinococcus granulosus | TTCAGAGTCGTAGGTATGTTGGTTT | 709 | AATTATACCCACAACAGCATAAAGC | 722 |
| 781 | Echinococcus granulosus | ATCTGTTAGGTTTGAGGCTTGTTTT | 724 | TTAGTCTCACATAACACACACACCA | 725 |
| 782 | Echinococcus granulosus | TTGCCGATTTGTTGAAGTTAGTAAT | 701 | AGCTAAAAATCATAACACAGAAAGCA | 726 |
| 783 | Echinococcus granulosus | GATCTGTTAGGTTTGAGGCTTGTTT | 727 | AGTCTCACATAACACACACACCAAG | 728 |
| 784 | Echinococcus granulosus | GTATTCCTTGTTGTGTACTGGTTGG | 729 | ATGGGAATAATAATCAACTTCACCA | 697 |
| 785 | Echinococcus granulosus | GGTATTCCTTGTTGTGTACTGGTTG | 730 | ATGGGAATAATAATCAACTTCACCA | 697 |
| 786 | Echinococcus granulosus | TACTTTCAGAGTCGTAGGTATGTTGG | 731 | ATAACCACACACATAAAACAAGCCT | 720 |
| 787 | Echinococcus granulosus | GCTTTCTGTGTTATGATTTTTAGCTG | 699 | ATAACCACACACATAAAACAAGCCT | 720 |
| 788 | Echinococcus granulosus | GTCGTAGGTATGTTGGTTTATTTGG | 732 | CAATTAAATTATACCCACAACAGCAT | 733 |
| 789 | Echinococcus granulosus | TTTCTGTGTTATGATTTTTAGCTGC | 715 | TGGGAATAATAATCAACTTCACCAG | 734 |
| 790 | Echinococcus granulosus | TTGCCGATTTGTTGAAGTTAGTAAT | 701 | TAAAACAAGCCTCAAACCTAACAGA | 735 |
| 791 | Echinococcus granulosus | TTGCCGATTTGTTGAAGTTAGTAAT | 701 | AAAACAAGCCTCAAACCTAACAGAT | 736 |
| 792 | Echinococcus granulosus | TTGGCGATTTGTTGAAGTTAGTAAT | 701 | ATAAAACAAGCCTCAAACCTAACAG | 737 |
| 793 | Echinococcus granulosus | TGCTTTCTGTGTTATGATTTTTAGC | 738 | ATGGGAATAATAATCAACTTCACCA | 697 |
| 794 | Echinococcus granulosus | ACTTTCAGAGTCGTAGGTATGTTGG | 739 | AAAACAAGCCTCAAACCTAACAGAT | 736 |
| 795 | Echinococcus granulosus | ACTTTCAGAGTCGTAGGTATGTTGG | 739 | ATAAAACAAGCCTCAAACCTAACAG | 737 |
| 796 | Echinococcus granulosus | TCTGTTAGGTTTGAGGCTTGTTTTA | 740 | CGATTAGTCTCACATAACACACACA | 741 |
| 797 | Echinococcus granulosus | GTTTGCCGATTTGTTGAAGTTAGTA | 742 | ATAAAACAAGCCTCAAACCTAACAG | 737 |
| 798 | Echinococcus granulosus | CTTCGAGACTTCTTTAGAAATGATCC | 743 | ACTTTGAATCATCATCTTTTTCTTCC | 744 |
| 799 | Echinococcus granulosus | TCTGTTAGGTTTGAGGCTTGTTTTA | 740 | ACTACTAATTCACTTTCAGCCTCCC | 745 |
| 800 | Echinococcus granulosus | ATCTGTTAGGTTTGAGGCTTGTTTT | 724 | ACTACTAATTCACTTTCAGCCTCCC | 745 |
| 801 | Ehrlichia canis | TTGAAATGAATGAACAAGAAAGACA | 746 | ATTGTAACAGGGTTATCAGCTATGC | 747 |
| 802 | Ehrlichia canis | TTGAAATGAATGAACAAGAAAGACA | 746 | TTATCAGCTATGCTTATGGAGTGTG | 748 |
| 803 | Ehrlichia canis | TTGAAATGAATGAACAAGAAAGACA | 746 | GGTTATCAGCTATGCTTATGGAGTG | 749 |
| 804 | Ehrlichia canis | TTGAAATGAATGAACAAGAAAGACA | 746 | TGTAACAGGGTTATCAGCTATGCTT | 750 |
| 805 | Ehrlichia canis | TTGAAATGAATGAACAAGAAAGACA | 746 | GGGTTATCAGCTATGCTTATGGAGT | 751 |
| 806 | Ehrlichia canis | TTGAAATGAATGAACAAGAAAGACA | 746 | AGGGTTATCAGCTATGCTTATGGAG | 752 |
| 807 | Ehrlichia canis | TTATATATGCGCAGGTATTGGTACTG | 753 | TAGTGTTACTGTTGCAAATTGTGGT | 754 |
| 808 | Ehrlichia canis | TCTTGAAATGAATGAACAAGAAAGA | 755 | ATTGTAACAGGGTTATCAGCTATGC | 747 |
| 809 | Ehrlichia canis | TCTTGAAATGAATGAACAAGAAAGA | 755 | TTATCAGCTATGCTTATGGAGTGTG | 748 |
| 810 | Ehrlichia canis | TCTTGAAATGAATGAACAAGAAAGA | 755 | GGTTATCAGCTATGCTTATGGAGTG | 749 |
| 811 | Ehrlichia canis | TCTTGAAATGAATGAACAAGAAAGA | 755 | TGTAACAGGGTTATCAGCTATGCTT | 750 |
| 812 | Ehrlichia canis | TGAAATGAATGAACAAGAAAGACAA | 756 | ATTGTAACAGGGTTATCAGCTATGC | 747 |
| 813 | Ehrlichia canis | TGAAATGAATGAACAAGAAAGACAA | 756 | TTATCAGCTATGCTTATGGAGTGTG | 748 |
| 814 | Ehrlichia canis | TGAAATGAATGAACAAGAAAGACAA | 756 | GGTTATCAGCTATGCTTATGGAGTG | 749 |
| 815 | Ehrlichia canis | TGAAATGAATGAACAAGAAAGACAA | 756 | TGTAACAGGGTTATCAGCTATGCTT | 750 |
| 816 | Ehrlichia canis | TGAAATGAATGAACAAGAAAGACAA | 756 | GGGTTATCAGCTATGCTTATGGAGT | 751 |
| 817 | Ehrlichia canis | TGAAATGAATGAACAAGAAAGACAA | 756 | AGGGTTATCAGCTATGCTTATGGAG | 752 |
| 818 | Ehrlichia canis | CCTTATATATGCGCAGGTATTGGTA | 757 | TAGTGTTACTGTTGCAAATTGTGGT | 754 |
| 819 | Ehrlichia canis | TATATATGCGCAGGTATTGGTACTG | 758 | TAGTGTTACTGTTGCAAATTGTGGT | 754 |
| 820 | Ehrlichia canis | ATATATGCGCAGGTATTGGTACTGA | 759 | TAGTGTTACTGTTGCAAATTGTGGT | 754 |
| 821 | Ehrlichia canis | CCTTATATATGCGCAGGTATTGGTA | 757 | TTAGTGTTACTGTTGCAAATTGTGG | 760 |
| 822 | Ehrlichia canis | TATATATGCGCAGGTATTGGTACTG | 758 | TTAGTGTTACTGTTGCAAATTGTGG | 760 |
| 823 | Ehrlichia canis | ATATATGCGCAGGTATTGGTACTGA | 759 | TTAGTGTTACTGTTGCAAATTGTGG | 760 |
| 824 | Ehrlichia canis | CCTTATATATGCGCAGGTATTGGTA | 757 | AGTGTTACTGTTGCAAATTGTGGT | 761 |
| 825 | Ehrlichia canis | TATATATGCGCAGGTATTGGTACTG | 758 | AGTGTTACTGTTGCAAATTGTGGT | 761 |
| 826 | Ehrlichia canis | TTATATATGCGCAGGTATTGGTACTG | 753 | TATGCCAAAGTGACACACATTTAGT | 762 |
| 827 | Ehrlichia canis | GCAGGTATTGGTACTGATTTGATTT | 763 | TGACACACATTTAGTGTTACTGTTGC | 764 |
| 828 | Ehrlichia canis | ATATATGCGCAGGTATTGGTACTG | 765 | TAGTGTTACTGTTGCAAATTGTGGT | 754 |
| 829 | Ehrlichia canis | CCTTATATATGCGCAGGTATTGGTA | 757 | AGTTCTATGCCAAAGTGACACACAT | 766 |
| 830 | Ehrlichia canis | TATATATGCGCAGGTATTGGTACTG | 758 | TCTATGCCAAAGTGACACACATTTA | 767 |
| 831 | Ehrlichia canis | ATATATGCGCAGGTATTGGTACTGA | 759 | TCTATGCCAAAGTGACACACATTTA | 767 |
| 832 | Ehrlichia canis | GCAGGTATTGGTACTGATTTGATTT | 763 | TAGTGTTACTGTTGCAAATTGTGGT | 754 |
| 833 | Ehrlichia canis | AAAACCTAACATTACAAGCGACAAC | 768 | ACATTTTACAAGGTACTTTTTCACCC | 769 |
| 834 | Ehrlichia canis | CTTTTTCACCTTATTTGTGCAGG | 770 | CAGGGATGTCTCTAAATTCATTACC | 771 |
| 835 | Ehrlichia canis | TTTTTCACCTTATATTTGTGCAGGT | 772 | CAGGGATGTCTCTAAATTCATTACC | 771 |
| 836 | Ehrlichia canis | CCTTATATATGCGCAGGTATTGGTA | 757 | TATGCCAAAGTGACACACATTTAGT | 762 |
| 837 | Ehrlichia canis | TATATATGCGCAGGTATTGGTACTG | 758 | TATGCCAAAGTGACACACATTTAGT | 762 |
| 838 | Ehrlichia canis | ATATATGCGCAGGTATTGGTACTGA | 759 | TATGCCAAAGTGACACACATTTAGT | 762 |
| 839 | Ehrlichia canis | ATAGAAACAAGCAAGGGTAGGAGTT | 773 | GTTGTCGCTTGTAATGTTAGGTTTTA | 774 |
| 840 | Ehrlichia canis | AGAAACAAGCAAGGGTAGGAGTTAT | 775 | GTTGTCGCTTGTAATGTTAGGTTTTA | 774 |
| 841 | Ehrlichia canis | GCATAGCTGATAACCCTGTTACAATA | 776 | TGAAGAGCTTGTTATCTACTGGCTT | 777 |
| 842 | Ehrlichia canis | TGAGTGCTAAATGTGAGGATTTTATC | 778 | GACCTTCATGTCAAAAAGTGGTAAG | 779 |
| 843 | Ehrlichia canis | TGCTAAATGTGAGGATTTTATCTTTG | 780 | GACCTTCATGTCAAAAAGTGGTAAG | 779 |
| 844 | Ehrlichia canis | CATAAGCATAGCTGATAACCCTGTT | 781 | ATAACTCCTACCCTTGCTTGTTTCTA | 782 |
| 845 | Ehrlichia canis | TAGAAACAAGCAAGGGTAGGAGTTAT | 783 | GTTGTCGCTTGTAATGTTAGGTTTT | 784 |
| 846 | Ehrlichia canis | GATGAGTGCTAAATGTGAGGATTTTA | 785 | GACCTTCATGTCAAAAAGTGGTAAG | 779 |
| 847 | Ehrlichia canis | CATAAGCATAGCTGATAACCCTGTT | 781 | TAACTCCTACCCTTGCTTGTTTCTAT | 786 |
| 848 | Ehrlichia canis | CATAAGCATAGCTGATAACCCTGTT | 781 | ACTCCTACCCTTGCTTGTTTCTATTA | 787 |
| 849 | Ehrlichia canis | AATAGAAACAAGCAAGGGTAGGAGT | 788 | GTTGTCGCTTGTAATGTTAGGTTTTA | 774 |
| 850 | Ehrlichia canis | TAATAGAAACAAGCAAGGGTAGGAGT | 789 | GTTGTCGCTTGTAATGTTAGGTTTT | 784 |
| 851 | Feline coronavirus | CGTGCTTCAAAAATTAGGTGTTACT | 790 | CATAGAAATTTGTCACATCACCCTT | 791 |
| 852 | Feline coronavirus | AGTTGTGAAGTGACTTTTCTGAACAC | 792 | TTCAAGGTCAACAAGGGTCTTATTA | 793 |
| 853 | Feline coronavirus | TACGAGTTGTGAAGTGACTTTTCTG | 794 | ATTCAAGGTCAACAAGGGTCTTATTA | 795 |
| 854 | Feline coronavirus | TTGTGAAGTGACTTTTCTGAACACTA | 796 | TTCAAGGTCAACAAGGGTCTTATTA | 793 |
| 855 | Feline coronavirus | AGTTGTGAAGTGACTTTTCTGAACAC | 792 | CTATGGTAGTGAGGTTGTCAGCTCT | 797 |
| 856 | Feline coronavirus | TTGTGAAGTGACTTTTCTGAACACTA | 796 | CTATGGTAGTGAGGTTGTCAGCTCT | 797 |
| 857 | Feline coronavirus | TACGAGTTGTGAAGTGACTTTTCTG | 794 | TCTAATTGGTCTATTTCTGCAGTGAG | 798 |
| 858 | Feline coronavirus | AGTTGTGAAGTGACTTTTCTGAACAC | 792 | CAGCTCTTTGCTCTAATTGGTCTAT | 799 |
| 859 | Feline coronavirus | AGTTGTGAAGTGACTTTTCTGAACAC | 792 | TATGGTAGTGAGGTTGTCAGCTCTT | 800 |
| 860 | Feline coronavirus | TACGAGTTGTGAAGTGACTTTTCTG | 794 | TGCAGTGAGGTTTAACTTAGTCTGAT | 801 |
| 861 | Feline coronavirus | TACGAGTTGTGAAGTGACTTTTCTG | 794 | TCTGCAGTGAGGTTTAACTTAGTCTG | 802 |
| 862 | Feline coronavirus | TACGAGTTGTGAAGTGACTTTTCTG | 794 | CTGCAGTGAGGTTTAACTTAGTCTGA | 803 |
| 863 | Feline coronavirus | TACGAGTTGTGAAGTGACTTTTCTG | 794 | TTTCTGCAGTGAGGTTTAACTTAGTC | 804 |
| 864 | Feline coronavirus | AGTTGTGAAGTGACTTTTCTGAACAC | 792 | TGCAGTGAGGTTTAACTTAGTCTGA | 805 |
| 865 | Feline coronavirus | TACGAGTTGTGAAGTGACTTTTCTG | 794 | AGCTCTTTGCTCTAATTGGTCTATTT | 806 |
| 866 | Feline coronavirus | TACGAGTTGTGAAGTGACTTTTCTG | 794 | GCTGTTTGCTCTAATTGGTCTATTTC | 807 |
| 867 | Feline coronavirus | CACTACATATACGACCTTTCAGGAGA | 808 | TTCAAGGTCAACAAGGGTCTTATTA | 793 |
| 868 | Feline coronavirus | AGTTGTGAAGTGACTTTTCTGAACAC | 792 | TTTGCTCTAATTGGTCTATTTCTGC | 809 |
| 869 | Feline coronavirus | CACAACACCTGAATTAAACTTACAGC | 810 | TTCAAGGTCAACAAGGGTCTTATTA | 793 |
| 870 | Feline coronavirus | TTGTGAAGTGACTTTTCTGAACACTA | 796 | TTTGCTCTAATTGGTCTATTTCTGC | 809 |
| 871 | Feline coronavirus | ACGAACTAAACGTATGCTTGTTTTC | 811 | GAAGAACTAGAAAGATCACTCGCAG | 812 |
| 872 | Feline coronavirus | CACTACATATACGACCTTTCAGGAGA | 808 | CTATGGTAGTGAGGTTGTCAGCTCT | 797 |
| 873 | Feline coronavirus | TACATATACGACCTTTCAGGAGATTG | 813 | CTATGGTAGTGAGGTTGTCAGCTCT | 797 |
| 874 | Feline coronavirus | TACGAGTTGTGAAGTGACTTTTCTG | 794 | TCTTTGCTCTAATTGGTCTATTTCTG | 814 |
| 875 | Feline coronavirus | ACGAACTAAACGTATGCTTGTTTTC | 811 | AGAAGAACTAGAAAGATCACTCGCA | 815 |
| 876 | Feline coronavirus | TACATATACGACCTTTCAGGAGATTG | 813 | TATGGTAGTGAGGTTGTCAGCTCTT | 800 |
| 877 | Feline coronavirus | ACATATACGACCTTTCAGGAGATTG | 816 | TCTAATTGGTCTATTTCTGCAGTGAG | 798 |
| 878 | Feline coronavirus | ATATACGACCTTTCAGGAGATTGTG | 817 | TCTAATTGGTCTATTTCTGCAGTGAG | 798 |
| 879 | Feline coronavirus | CATATACGACCTTTCAGGAGATTGT | 818 | TCTAATTGGTCTATTTCTGCAGTGAG | 798 |
| 880 | Feline coronavirus | CACTACATATACGACCTTTCAGGAGA | 808 | TATGGTAGTGAGGTTGTCAGCTCTT | 800 |
| 881 | Feline coronavirus | TACATATACGACCTTTCAGGAGATTG | 813 | CAGCTCTTTGCTCTAATTGGTCTAT | 799 |
| 882 | Feline coronavirus | ACGAACTAAACGTATGCTTGTTTTC | 811 | TGTATAGCAGGAAAACCCTAGAAGA | 819 |
| 883 | Feline coronavirus | CATATACGACCTTTCAGGAGATTGT | 818 | TGCAGTGAGGTTTAACTTAGTCTGAT | 801 |
| 884 | Feline coronavirus | ATATACGACCTTTCAGGAGATTGTG | 817 | TGCAGTGAGGTTTAACTTAGTCTGAT | 801 |
| 885 | Feline coronavirus | ACATATACGACCTTTCAGGAGATTG | 816 | TGCAGTGAGGTTTAACTTAGTCTGAT | 801 |
| 886 | Feline coronavirus | CACTACATATACGACCTTTCAGGAGA | 808 | TGCAGTGAGGTTTAACTTAGTCTGA | 805 |
| 887 | Feline coronavirus | ATATACGACCTTTCAGGAGATTGTG | 817 | TCTGCAGTGAGGTTTAACTTAGTCTG | 802 |
| 888 | Feline coronavirus | ACATATACGACCTTTCAGGAGATTG | 816 | TCTGCAGTGAGGTTTAACTTAGTCTG | 802 |
| 889 | Feline coronavirus | CATATACGACCTTTCAGGAGATTGT | 818 | TCTGCAGTGAGGTTTAACTTAGTCTG | 802 |
| 890 | Feline coronavirus | ACATATACGACCTTTCAGGAGATTG | 816 | CTGCAGTGAGGTTTAACTTAGTCTGA | 803 |
| 891 | Feline coronavirus | ATATACGACCTTTCAGGAGATTGTG | 817 | CTGCAGTGAGGTTTAACTTAGTCTGA | 803 |
| 892 | Feline coronavirus | CATATACGACCTTTCAGGAGATTGT | 818 | CTGCAGTGAGGTTTAACTTAGTCTGA | 803 |
| 893 | Feline coronavirus | TACATATACGACCTTTCAGGAGATTG | 813 | TGCAGTGAGGTTTAACTTAGTCTGA | 805 |
| 894 | Feline coronavirus | ATATACGACCTTTCAGGAGATTGTG | 817 | TTTCTGCAGTGAGGTTTAACTTAGTC | 804 |
| 895 | Feline coronavirus | CATATACGACCTTTCAGGAGATTGT | 818 | TTTCTGCAGTGAGGTTTAACTTAGTC | 804 |
| 896 | Feline coronavirus | ACATATACGACCTTTCAGGAGATTG | 816 | TTTCTGCAGTGAGGTTTAACTTAGTC | 804 |
| 897 | Feline coronavirus | ATATACGACCTTTCAGGAGATTGTG | 817 | AGCTCTTTGCTCTAATTGGTCTATTT | 806 |
| 898 | Feline coronavirus | CATATACGACCTTTCAGGAGATTGT | 818 | AGCTCTTTGCTCTAATTGGTCTATTT | 806 |
| 899 | Feline coronavirus | ACATATACGACCTTTCAGGAGATTG | 816 | AGCTCTTTGCTCTAATTGGTCTATTT | 806 |
| 900 | Feline coronavirus | CATATACGACCTTTCAGGAGATTGT | 818 | GCTCTTTGCTCTAATTGGTCTATTTC | 807 |
| 901 | Feline herpesvirus | ATATAACTTAAGAGGAGCGGCTGAT | 820 | CTGCAAGAAAGAGTCTTGTAGGAAG | 821 |
| 902 | Feline herpesvirus | TCTGGATCGTACTATTCTTAGTCGG | 822 | GGACACATATAAAAAGTCGATGGTC | 823 |
| 903 | Feline herpesvirus | TAATGGTCTTCTCCCATGTCTACAG | 824 | TACCTGTACATTATAGGGTTTATCCG | 825 |
| 904 | Feline herpesvirus | GTCTTCTCCCATGTCTACAGATAGC | 826 | TACCTGTACATTATAGGGTTTATCCG | 825 |
| 905 | Feline herpesvirus | GCTAATGGTCTTCTCCCATGTCTAC | 827 | TATAGGGTTTATCCGGAATAGTAACG | 828 |
| 906 | Feline herpesvirus | GCTAATGGTCTTCTCCCATGTCTAC | 827 | GTTTATCCGGAATAGTAACGGTCATA | 829 |
| 907 | Feline herpesvirus | GCTAATGGTCTTCTCCCATGTCTAC | 827 | TTTATCCGGAATAGTAACGGTCATAG | 830 |
| 908 | Feline herpesvirus | GGTAATGGTCTTCTCCCATGTCTAC | 827 | ATAGTAACGGTCATAGCTTCTGGAAA | 831 |
| 909 | Feline herpesvirus | GCTAATGGTCTTCTCCCATGTCTAC | 827 | GAATAGTAACGGTCATAGCTTCTGGA | 832 |
| 910 | Feline herpesvirus | CTAATGGTCTTCTCCCATGTCTACAG | 833 | TATAGGGTTTATCCGGAATAGTAACG | 828 |
| 911 | Feline herpesvirus | GCTAATGGTCTTCTCCCATGTCTAC | 827 | ACCTGTACATTATAGGGTTTATCCG | 834 |
| 912 | Feline herpesvirus | GCTAATGGTCTTCTCCCATGTCTAC | 827 | CATTATAGGGTTTATCCGGAATAGT | 835 |
| 913 | Feline herpesvirus | GCTAATGGTCTTCTCCCATGTCTAC | 827 | TTTATCCGGAATAGTAACGGTCATA | 836 |
| 914 | Feline herpesvirus | GCTAATGGTCTTCTCCCATGTCTAC | 827 | ATAGGGTTTATCCGGAATAGTAACG | 837 |
| 915 | Feline herpesvirus | GCTAATGGTCTTCTCCCATGTCTAC | 827 | TTATCCGGAATAGTAACGGTCATAG | 838 |
| 916 | Feline herpesvirus | GCTAATGGTCTTCTCCCATGTCTAC | 827 | GAATAGTAACGGTCATAGCTTCTGG | 839 |
| 917 | Feline herpesvirus | GCTAATGGTCTTCTCCCATGTCTAC | 827 | GGAATAGTAACGGTCATAGCTTCTG | 840 |
| 918 | Feline herpesvirus | GCTAATGGTCTTCTCCCATGTCTAC | 827 | GTTTATCCGGAATAGTAACGGTCAT | 841 |
| 919 | Feline herpesvirus | GCTAATGGTCTTCTCCCATGTCTAC | 827 | CGGAATAGTAACGGTCATAGCTTCT | 842 |
| 920 | Feline herpesvirus | GCTAATGGTCTTCTCCCATGTCTA | 843 | TATAGGGTTTATCCGGAATAGTAACG | 828 |
| 921 | Feline herpesvirus | GCTAATGGTCTTCTCCCATGTCTAC | 827 | TATCCGGAATAGTAACGGTCATAGC | 844 |
| 922 | Feline herpesvirus | GCTAATGGTCTTCTCCCATGTCTA | 843 | GTTTATCCGGAATAGTAACGGTCATA | 829 |
| 923 | Feline herpesvirus | GCTAATGGTCTTCTCCCATGTCTA | 843 | TTTATCCGGAATAGTAACGGTCATAG | 830 |
| 924 | Feline herpesvirus | GCTAATGGTCTTCTCCCATGTCTAC | 827 | AATAGTAACGGTCATAGCTTCTGGA | 845 |
| 925 | Feline herpesvirus | GCTAATGGTCTTCTCCCATGTCTAC | 827 | TAGTAACGGTCATAGCTTCTGGAAA | 846 |
| 926 | Feline herpesvirus | GCTAATGGTCTTCTCCCATGTCTAC | 827 | AGTAACGGTCATAGCTTCTGGAAAA | 847 |
| 927 | Feline herpesvirus | GCTAATGGTCTTCTCCCATGTCTA | 843 | ATAGTAACGGTCATAGCTTCTGGAAA | 831 |
| 928 | Feline herpesvirus | TAATGGTCTTCTCCCATGTCTACAG | 824 | CATTATAGGGTTTATCCGGAATAGTAA | 848 |
| 929 | Feline herpesvirus | TAATGGTCTTCTCCCATGTCTACAG | 824 | TTATAGGGTTTATCCGGAATAGTAACG | 849 |
| 930 | Feline herpesvirus | CTAATGGTCTTCTCCCATGTCTACAG | 833 | TTTATCCGGAATAGTAACGGTCATA | 836 |
| 931 | Feline herpesvirus | CTAATGGTCTTCTCCCATGTCTACAG | 833 | ATAGGGTTTATCCGGAATAGTAACG | 837 |
| 932 | Feline herpesvirus | CTAATGGTCTTCTCCCATGTCTACAG | 833 | TTATCCGGAATAGTAACGGTCATAG | 838 |
| 933 | Feline herpesvirus | CTAATGGTCTTCTCCCATGTCTACAG | 833 | GAATAGTAACGGTCATAGCTTCTGG | 839 |
| 934 | Feline herpesvirus | CTAATGGTCTTCTCCCATGTCTACAG | 833 | GGAATAGTAACGGTCATAGCTTCTG | 840 |
| 935 | Feline herpesvirus | CTAATGGTCTTCTCCCATGTCTACAG | 833 | GTTTATCCGGAATAGTAACGGTCAT | 841 |
| 936 | Feline herpesvirus | CTAATGGTCTTCTCCCATGTCTACAG | 833 | CGGAATAGTAACGGTCATAGCTTCT | 842 |
| 937 | Feline herpesvirus | CTAATGGTCTTCTCCCATGTCTACAG | 833 | AATAGTAACGGTCATAGCTTCTGGA | 845 |
| 938 | Feline herpesvirus | CTAATGGTCTTCTCCCATGTCTACAG | 833 | TAGTAACGGTCATAGCTTCTGGAAA | 846 |
| 939 | Feline herpesvirus | GCTAATGGTCTTCTCCCATGTCTAC | 827 | TAGGGTTTATCCGGAATAGTAACG | 850 |
| 940 | Feline herpesvirus | GCTAATGGTCTTCTCCCATGTCTA | 843 | ACCTGTACATTATAGGGTTTATCCG | 834 |
| 941 | Feline herpesvirus | GCTAATGGTCTTCTCCCATGTCTAC | 827 | GGTTTATCCGGAATAGTAACGGTC | 851 |
| 942 | Feline herpesvirus | GCTAATGGTCTTCTCCCATGTCTA | 843 | TTTATCCGGAATAGTAACGGTCATA | 836 |
| 943 | Feline herpesvirus | GCTAATGGTCTTCTCCCATGTCTA | 843 | ATAGGGTTTATCCGGAATAGTAACG | 837 |
| 944 | Feline herpesvirus | GCTAATGGTCTTCTCCCATGTCTA | 843 | TTATCCGGAATAGTAACGGTCATAG | 838 |
| 945 | Feline herpesvirus | GCTAATGGTCTTCTCCCATGTCTA | 843 | GGAATAGTAACGGTCATAGCTTCTG | 840 |
| 946 | Feline herpesvirus | GCTAATGGTCTTCTCCCATGTCTA | 843 | GAATAGTAACGGTCATAGCTTCTGG | 839 |
| 947 | Feline herpesvirus | GCTAATGGTCTTCTCCCATGTCTA | 843 | GTTTATCCGGAATAGTAACGGTCAT | 841 |
| 948 | Feline herpesvirus | GCTAATGGTCTTCTCCCATGTCTAC | 827 | TTTATCCGGAATAGTAACGGTCAT | 852 |
| 949 | Feline herpesvirus | GCTAATGGTCTTCTCCCATGTCTAC | 827 | CGGAATAGTAACGGTCATAGCTTC | 853 |
| 950 | Feline herpesvirus | GCTAATGGTCTTCTCCCATGTCTAC | 827 | GTTTATCCGGAATAGTAACGGTCA | 854 |
| 951 | Feline immunodeficiency | AGGTAGGAGAGATTCTACAGCAACA | 855 | GTTCTCGTCCTGTAGATACGTTAGC | 856 |
| | virus | | | | |
| 952 | Feline immunodeficiency | TCTGCAAATTTAACACCTACTGACA | 857 | TGCTGCAGTAAAATAAGGTAATGGT | 858 |
| | virus | | | | |
| 953 | Feline immunodeficiency | TAGATGGAAAACAAGCTGAATATGAC | 859 | AGATTACACAGATACACGACAGGGT | 860 |
| | virus | | | | |
| 954 | Feline immunodeficiency | ATTCCCATAGATAAAGATTATCAGCCT | 861 | ATTTCTGGATGTTGTTTTCTAAAAGG | 862 |
| | virus | | | | |
| 955 | Feline immunodeficiency | TATTCCCATAGATAAAGATTATCAGCCT | 863 | TTCTGGATGTTGTTTTCTAAAAGGT | 864 |
| | virus | | | | |
| 956 | Feline immunodeficiency | CTATTCCCATAGATAAAGATTATCAGCC | 865 | TCTGGATGTTGTTTTCTAAAAGGTT | 866 |
| | virus | | | | |
| 957 | Feline immunodeficiency | CTATTCCCATAGATAAAGATTATCAGCC | 865 | TTCTGGATGTTGTTTTCTAAAAGGT | 864 |
| | virus | | | | |
| 958 | Feline immunodeficiency | TATTCCCATAGATAAAGATTATCAGCCT | 863 | TCTGGATGTTGTTTTCTAAAAGGTT | 866 |
| | virus | | | | |
| 959 | Feline immunodeficiency | CTATTCCCATAGATAAAGATTATCAGCC | 865 | TTTCTGGATGTTGTTTTCTAAAAGG | 867 |
| | virus | | | | |
| 960 | Feline immunodeficiency | TATTCCCATAGATAAAGATTATCAGCCT | 863 | TTTCTGGATGTTGTTTTCTAAAAGG | 867 |
| | virus | | | | |
| 961 | Feline immunodeficiency | TTCCCATAGATAAAGATTATCAGCCT | 868 | TATTTCTGGATGTTGTTTTCTAAAAGG | 869 |
| | virus | | | | |
| 962 | Feline immunodeficiency | GTGATGCTTATTTCACTATTCCCATA | 870 | TATTTCTGGATGTTGTTTTCTAAAAGG | 869 |
| | virus | | | | |
| 963 | Feline immunodeficiency | TAGTGATGCTTATTTCACTATTCCCA | 871 | TATTTCTGGATGTTGTTTTCTAAAAGG | 869 |
| | virus | | | | |
| 964 | Feline immunodeficiency | ACTAGATATGTATGGAAATGCCTGC | 872 | TGTTTATGTTCTTTAGGAGCTAAATCTG | 873 |
| | virus | | | | |
| 965 | Feline immunodeficiency | ATTCCCATAGATAAAGATTATCAGCCT | 861 | TTCTGGATGTTGTTTTCTAAAAGGT | 864 |
| | virus | | | | |
| 966 | Feline immunodeficiency | ATTCCCATAGATAAAGATTATCAGCCT | 861 | TCTGGATGTTGTTTTCTAAAAGGTT | 866 |
| | virus | | | | |
| 967 | Feline immunodeficiency | TATTCCCATAGATAAAGATTATCAGCC | 874 | TTCTGGATGTTGTTTTCTAAAAGGT | 864 |
| | virus | | | | |
| 968 | Feline immunodeficiency | TATTCCCATAGATAAAGATTATCAGCC | 874 | TCTGGATGTTGTTTTCTAAAAGGTT | 866 |
| | virus | | | | |
| 969 | Feline immunodeficiency | ATTCCCATAGATAAAGATTATCAGCCT | 861 | TTTCTGGATGTTGTTTTCTAAAAGG | 867 |
| | virus | | | | |
| 970 | Feline immunodeficiency | TTCCCATAGATAAAGATTATCAGCCT | 868 | ATTTCTGGATGTTGTTTTCTAAAAGG | 862 |
| | virus | | | | |
| 971 | Feline immunodeficiency | TATTCCCATAGATAAAGATTATCAGCC | 874 | TTTCTGGATGTTGTTTTCTAAAAGG | 867 |
| | virus | | | | |
| 972 | Feline immunodeficiency | ATTCCCATAGATAAAGATTATCAGCC | 875 | ATTTCTGGATGTTGTTTCTAAAAGG | 862 |
| | virus | | | | |
| 973 | Feline immunodeficiency | GGATTACCTCAAAAAGCTAGAGTAACA | 876 | GGCATTTCCATACATATCTAGTTCC | 877 |
| | virus | | | | |
| 974 | Feline immunodeficiency | CCTTATACGGCGTTTACTTTACCTA | 878 | TATTTCTGGATGTTGTTTTCTAAAAGG | 869 |
| | virus | | | | |
| 975 | Feline immunodeficiency | GATGCTTATTTCACTATTCCCATAGAT | 879 | TTCTGGATGTTGTTTTCTAAAAGGT | 864 |
| | virus | | | | |
| 976 | Feline immunodeficiency | GATGCTTATTTCACTATTCCCATAGAT | 879 | TCTGGATGTTGTTTTCTAAAAGGTT | 866 |
| | virus | | | | |
| 977 | Feline immunodeficiency | GTGATGCTTATTTCACTATTCCCATA | 870 | ATTTCTGGATGTTGTTTTCTAAAAGG | 862 |
| | virus | | | | |
| 978 | Feline immunodeficiency | GATGCTTATTTCACTATTCCCATAGAT | 879 | TTTCTGGATGTTGTTTTCTAAAAGG | 867 |
| | virus | | | | |
| 979 | Feline immunodeficiency | GGATTACCTCAAAAAGCTAGAGTAACA | 876 | TTCTGGATGTTGTTTTCTAAAAGGT | 864 |
| | virus | | | | |
| 980 | Feline immunodeficiency | GGATTACCTCAAAAAGCTAGAGTAACA | 876 | TCTGGATGTTGTTTTCTAAAAGGTT | 866 |
| | virus | | | | |
| 981 | Feline immunodeficiency | TTCCCATAGATAAAGATTATCAGCCT | 868 | TTTCTGGATGTTGTTTTCTAAAAGGT | 880 |
| | virus | | | | |
| 982 | Feline immunodeficiency | TTCCCATAGATAAAGATTATCAGCCT | 868 | TTCTGGATGTTGTTTTCTAAAAGGTT | 881 |
| | virus | | | | |
| 983 | Feline immunodeficiency | CAGGATTACCTCAAAAAGCTAGAGTAA | 882 | GGCATTTCCATACATATCTAGTTCC | 877 |
| | virus | | | | |
| 984 | Feline immunodeficiency | GATGCTTATTTCACTATTCCCATAGA | 883 | ATTTCTGGATGTTGTTTTCTAAAAGG | 862 |
| | virus | | | | |
| 985 | Feline immunodeficiency | TTCCCATAGATAAAGATTATCAGCC | 884 | TATTTCTGGATGTTGTTTTCTAAAAGG | 869 |
| | virus | | | | |
| 986 | Feline immunodeficiency | ATTCCCATAGATAAAGATTATCAGCC | 875 | TTCTGGATGTTGTTTTCTAAAAGGT | 880 |
| | virus | | | | |
| 987 | Feline immunodeficiency | ATTCCCATAGATAAAGATTATCAGCC | 875 | TTCTGGATGTTGTTTTCTAAAAGGTT | 881 |
| | virus | | | | |
| 988 | Feline immunodeficiency | TGATGCTTATTTCACTATTCCCATAGA | 885 | TTTCTGGATGTTGTTTTCTAAAAGG | 867 |
| | virus | | | | |
| 989 | Feline immunodeficiency | TCCCATAGATAAAGATTATCAGCCT | 886 | TATTTCTGGATGTTGTTTTCTAAAAGG | 869 |
| | virus | | | | |
| 990 | Feline immunodeficiency | GGATTACCTCAAAAAGCTAGAGTAACA | 876 | TTTCTGGATGTTGTTTTCTAAAAGG | 867 |
| | virus | | | | |
| 991 | Feline immunodeficiency | AGTGATGCTTATTTCACTATTCCCATA | 887 | TTCTGGATGTTGTTTTCTAAAAGGT | 864 |
| | virus | | | | |
| 992 | Feline immunodeficiency | AGTGATGCTTATTTCACTATTCCCATA | 887 | TCTGGATGTTGTTTTCTAAAAGGTT | 866 |
| | virus | | | | |
| 993 | Feline immunodeficiency | TAGTGATGCTTATTTCACTATTCCCA | 871 | ATTTCTGGATGTTGTTTTCTAAAAGG | 862 |
| | virus | | | | |
| 994 | Feline immunodeficiency | GTGATGCTTATTTCACTATTCCCAT | 888 | TATTTCTGGATGTTGTTTTCTAAAAGG | 869 |
| | virus | | | | |
| 995 | Feline immunodeficiency | AGTGATGCTTATTTCACTATTCCCATA | 887 | TTTCTGGATGTTGTTTTCTAAAAGG | 867 |
| | virus | | | | |
| 996 | Feline immunodeficiency | AGTGATGCTTATTTCACTATTCCCAT | 889 | ATTTCTGGATGTTGTTTTCTAAAAGG | 862 |
| | virus | | | | |
| 997 | Feline immunodeficiency | CAGGATTACCTCAAAAAGCTAGAGTAA | 882 | TTCTGGATGTTGTTTTCTAAAAGGT | 864 |
| | virus | | | | |
| 998 | Feline immunodeficiency | CAGGATTACCTCAAAAAGCTAGAGTAA | 882 | TCTGGATGTTGTTTTCTAAAAGGTT | 866 |
| | virus | | | | |
| 999 | Feline immunodeficiency | GTGATGCTTATTTCACTATTCCCATA | 870 | TTTCTGGATGTTGTTTTCTAAAAGGT | 880 |
| | virus | | | | |
| 1000 | Feline immunodeficiency | GTGATGCTTATTTCACTATTCCCATA | 870 | TTCTGGATGTTGTTTTCTAAAAGGTT | 881 |
| | virus | | | | |
| 1001 | Feline leukemia virus | CCAAACCTAGTCTTACCTGATCAAA | 890 | CAGAGCCAACAGTCTTAGTTTTGT | 891 |
| 1002 | Feline leukemia virus | TTGCAATCTTAGGTAACTACAGCAAC | 892 | CGATTAAGACACAAAAATCAGAGGT | 893 |
| 1003 | Feline leukemia virus | ACAAGCTGACCATATCTGAAGTATCA | 894 | CGATTAAGACACAAAAATCAGAGGT | 893 |
| 1004 | Feline leukemia virus | CACAAGCTGACCATATCTGAAGTATC | 895 | CGATTAAGACACAAAAATCAGAGGT | 893 |
| 1005 | Feline leukemia virus | AACACAAGCTGACCATATCTGAAGTA | 896 | CGATTAAGACACAAAAATCAGAGGT | 893 |
| 1006 | Feline leukemia virus | AGCTGACCATATCTGAAGTATCAGG | 897 | GATTAAGACACAAAAATCAGAGGTCC | 898 |
| 1007 | Feline leukemia virus | TTGCAATCTTAGGTAACTACAGCAAC | 892 | GCCATAATTCGATTAAGACACAAAA | 899 |
| 1008 | Feline leukemia virus | TTAGTACAAGGGACATACCTAGCCTT | 900 | TGATACTTCAGATATGGTCAGCTTG | 901 |
| 1009 | Feline leukemia virus | TTTAGTACAAGGGACATACCTAGCCT | 902 | TGATACTTCAGATATGGTCAGCTTG | 901 |
| 1010 | Feline leukemia virus | TTAGTACAAGGGACATACCTAGCCTT | 900 | ACTTCAGATATGGTCAGCTTGTGTT | 903 |
| 1011 | Feline leukemia virus | TTTAGTACAAGGGACATACCTAGCCT | 902 | ACTTCAGATATGGTCAGCTTGTGTT | 903 |
| 1012 | Feline leukemia virus | GAGTAGTCAGGACAATAACTGTGAGG | 904 | TGATCAGGTAAGACTAGGTTTGGTC | 905 |
| 1013 | Feline leukemia virus | TACAAGGGACATACCTAGCCTTAAAT | 906 | TGATACTTCAGATATGGTCAGCTTG | 901 |
| 1014 | Feline leukemia virus | ACAAGGGACATACCTAGCCTTAAAT | 907 | GATACTTCAGATATGGTCAGCTTGTG | 908 |
| 1015 | Feline leukemia virus | CCAAACCTAGTCTTACCTGATCAAA | 890 | GGCTAGGTATGTCCCTTGTACTAAAT | 909 |
| 1016 | Feline leukemia virus | CCAAACCTAGTCTTACCTGATCAAA | 890 | AGGCTAGGTATGTCCCTTGTACTAAA | 910 |
| 1017 | Feline leukemia virus | TCCTCATGGGACTATATCACAGTAAA | 911 | TGATCAGGTAAGACTAGGTTTGGTC | 905 |
| 1018 | Feline leukemia virus | CCTCATGGGACTATATCACAGTAAAA | 912 | TGATCAGGTAAGACTAGGTTTGGTC | 905 |
| 1019 | Feline leukemia virus | GAGTAGTCAGGACAATAACTGTGAGG | 904 | TTTGATCAGGTAAGACTAGGTTTGG | 913 |
| 1020 | Feline leukemia virus | GTACAAGGGACATACCTAGCCTTAAA | 914 | TGATACTTCAGATATGGTCAGCTTG | 901 |
| 1021 | Feline leukemia virus | GTACAAGGGACATACCTAGCCTTAAA | 914 | ACTTCAGATATGGTCAGCTTGTGTT | 903 |
| 1022 | Feline leukemia virus | CCTAGTCTTACCTGATCAAAAACCC | 915 | AGGCTAGGTATGTCCCTTGTACTAAA | 910 |
| 1023 | Feline leukemia virus | CTAGTCTTACCTGATCAAAAACCCC | 916 | AGGCTAGGTATGTCCCTTGTACTAAA | 910 |
| 1024 | Feline leukemia virus | ATTTCACAAGGCATGGAAAATTAC | 917 | GAAACTTCTGCTGTTTCAGCTATATCC | 918 |
| 1025 | Feline leukemia virus | TCCTCATGGGACTATATCACAGTAAA | 911 | TTTGATCAGGTAAGACTAGGTTTGG | 913 |
| 1026 | Feline leukemia virus | CCTCATGGGACTATATCACAGTAAAA | 912 | TTTGATCAGGTAAGACTAGGTTTGG | 913 |
| 1027 | Feline leukemia virus | GGAGTAGTCAGGACAATAACTGTGAG | 919 | TGATCAGGTAAGACTAGGTTTGGTC | 905 |
| 1028 | Feline leukemia virus | TCCTCATGGGACTATATCACAGTAAA | 911 | TTGATCAGGTAAGACTAGGTTTGGT | 920 |
| 1029 | Feline leukemia virus | CCAAACCTAGTCTTACCTGATCAAA | 890 | TTGGTTGCTGTAGTTACCTAAGATTG | 921 |
| 1030 | Feline leukemia virus | CTTAGGTAACTACAGCAACCAAACAA | 922 | CGATTAAGACACAAAAATCAGAGGT | 893 |
| 1031 | Feline leukemia virus | GGAGTAGTCAGGACAATAACTGTGAG | 919 | TTTGATCAGGTAAGACTAGGTTTGG | 913 |
| 1032 | Feline leukemia virus | AGCTGACCATATCTGAAGTATCAGG | 897 | AATTCGATTAAGACACAAAAATCAGA | 923 |
| 1033 | Feline leukemia virus | CTAGTCTTACCTGATCAAAAACCCC | 916 | TTGGTTGCTGTAGTTACCTAAGATTG | 921 |
| 1034 | Feline leukemia virus | CCTAGTCTTACCTGATCAAAAACCC | 915 | TTGGTTGCTGTAGTTACCTAAGATTG | 921 |
| 1035 | Feline leukemia virus | CTAGTCTTACCTGATCAAAAACCCC | 916 | TTAAGGCTAGGTATGTCCCTTGTACT | 924 |
| 1036 | Feline leukemia virus | CCTAGTCTTACCTGATCAAAAACCC | 915 | TTAAGGCTAGGTATGTCCCTTGTACT | 924 |
| 1037 | Feline leukemia virus | CTTAGGTAACTACAGCAACCAAACAA | 922 | GCCATAATTCGATTAAGACACAAAA | 899 |
| 1038 | Feline leukemia virus | TTAGGTAACTACAGCAACCAAACAAA | 925 | CGATTAAGACACAAAAATCAGAGGT | 893 |
| 1039 | Feline leukemia virus | GGGACTATATCACAGTAAAAAGAGGG | 926 | TGATCAGGTAAGACTAGGTTTGGTC | 905 |
| 1040 | Feline leukemia virus | TAGGTAACTACAGCAACCAAACAAAC | 927 | CGATTAAGACACAAAAATCAGAGGT | 893 |
| 1041 | Feline leukemia virus | GGGACTATATCACAGTAAAAAGAGGG | 926 | TTTGATCAGGTAAGACTAGGTTTGG | 913 |
| 1042 | Feline leukemia virus | TAAAAAGAGGGAGTAGTCAGGACAAT | 928 | TGATCAGGTAAGACTAGGTTTGGTC | 905 |
| 1043 | Feline leukemia virus | TAGGTAACTACAGCAACCAAACAAAC | 927 | GCCATAATTCGATTAAGACACAAAA | 899 |
| 1044 | Feline leukemia virus | TAAAAAGAGGGAGTAGTCAGGACAAT | 928 | TTTGATCAGGTAAGACTAGGTTTGG | 913 |
| 1045 | Feline leukemia virus | GCTATTGCAGTAATGCCATTTCAC | 929 | GAAACTTCTGCTGTTTCAGCTATATCC | 918 |
| 1046 | Feline leukemia virus | TATAGTCCTAAGCCCAACCAATGTA | 930 | GTCCTGACTACTCCCTCTTTTTACTG | 931 |
| 1047 | Feline leukemia virus | ACAGTAAAAAGAGGGAGTAGTCAGGA | 932 | TGATCAGGTAAGACTAGGTTTGGTC | 905 |
| 1048 | Feline leukemia virus | AGTAAAAAGAGGGAGTAGTCAGGACA | 933 | TGATCAGGTAAGACTAGGTTTGGTC | 905 |
| 1049 | Feline leukemia virus | ACAGTAAAAAGAGGGAGTAGTCAGGA | 932 | TTTGATCAGGTAAGACTAGGTTTGG | 913 |
| 1050 | Feline leukemia virus | TATAGTCCTAAGCCCAACCAATGTA | 930 | ATTGTCCTGACTACTCCCTCTTTTTA | 934 |
| 1051 | Feline parvovirus | CAGTTAAAGGAAACATGGCTTTAGA | 935 | CTGCTGGAGTAAATGGCATAGTATT | 936 |
| 1052 | Feline parvovirus | AGATATCCAGAAGGAGATTGGATTC | 492 | CATTTGGATAAACTGGTGGTACATT | 493 |
| 1053 | Feline parvovirus | ATGTATTGCTACCAACAGATCCAAT | 494 | GTGCATTTACATGAAGTCTTGGTTT | 503 |
| 1054 | Feline parvovirus | AGATATCCAGAAGGAGATTGGATTC | 492 | ATTCTTTATCCCAAATTTGACCATT | 937 |
| 1055 | Feline parvovirus | ACAGGAAGATATCCAGAAGGAGATT | 498 | CATTTGGATAAACTGGTGGTACATT | 493 |
| 1056 | Feline parvovirus | GAAGATATCCAGAAGGAGATTGGAT | 499 | CATTTGGATAAACTGGTGGTACATT | 493 |
| 1057 | Feline parvovirus | AAGATATCCAGAAGGAGATTGGATT | 497 | CATTTGGATAAACTGGTGGTACATT | 493 |
| 1058 | Feline parvovirus | CAGGAGAAACACCTGAGAGATTTAC | 515 | CATTTGGATAAACTGGTGGTACATT | 493 |
| 1059 | Feline parvovirus | AGGAGAAACACCTGAGAGATTTACA | 516 | CATTTGGATAAACTGGTGGTACATT | 493 |
| 1060 | Feline parvovirus | ACAGGAGAAACACCTGAGAGATTTA | 517 | CATTTGGATAAACTGGTGGTACATT | 493 |
| 1061 | Feline parvovirus | CAAGATATGCATTTGGTAGACAACA | 502 | CATTTGGATAAACTGGTGGTACATT | 493 |
| 1062 | Feline parvovirus | ACAGGAAGATATCCAGAAGGAGATT | 498 | GTGCATTTACATGAAGTCTTGGTTT | 503 |
| 1063 | Feline parvovirus | ACAGGAAGATATCCAGAAGGAGATT | 498 | ATTCTTTATCCCAAATTTGACCATT | 937 |
| 1064 | Feline parvovirus | GAAGATATCCAGAAGGAGATTGGAT | 499 | ATTCTTTATCCCAAATTTGACCATT | 937 |
| 1065 | Feline parvovirus | AAGATATCCAGAAGGAGATTGGATT | 497 | ATTCTTTATCCCAAATTTGACCATT | 937 |
| 1066 | Feline parvovirus | AGGAGAAACACCTGAGAGATTTACA | 516 | ATTCTTTATCCCAAATTTGACCATT | 937 |
| 1067 | Feline parvovirus | CAGGAGAAACACCTGAGAGATTTAC | 515 | ATTCTTTATCCCAAATTTGACCATT | 937 |
| 1068 | Feline parvovirus | ACAGGAGAAACACCTGAGAGATTTA | 517 | ATTCTTTATCCCAAATTTGACCATT | 937 |
| 1069 | Feline parvovirus | CAAGATATGCATTTGGTAGACAACA | 502 | ATTCTTTATCCCAAATTTGACCATT | 937 |
| 1070 | Feline parvovirus | GCTACTATTATGAGACCAGCTGAGG | 506 | CATGTTGTCTACCAAATGCATATCT | 514 |
| 1071 | Feline parvovirus | ACTATTATGAGACCAGCTGAGGTTG | 511 | AATCTCCTTCTGGATATCTTCCTGT | 512 |
| 1072 | Feline parvovirus | GCTACTATTATGAGACCAGCTGAGG | 506 | AATCTCCTTCTGGATATCTTCCTGT | 512 |
| 1073 | Feline parvovirus | GCTACTATTATGAGACCAGCTGAGG | 506 | ATCCAATCTCCTTCTGGATATCTTC | 513 |
| 1074 | Feline parvovirus | ACTATTATGAGACCAGCTGAGGTTG | 511 | TAAATCTCTCAGGTGTTTCTCCTGT | 938 |
| 1075 | Feline parvovirus | GTGCACCATATTATTCTTTTGAAGC | 939 | AATCTCCTTCTGGATATCTTCCTGT | 512 |
| 1076 | Feline parvovirus | ACTATTATGAGACCAGCTGAGGTTG | 511 | TGTTGTCTACCAAATGCATATCTTG | 510 |
| 1077 | Feline parvovirus | GCTACTATTATGAGACCAGCTGAGG | 506 | AATCCAATCTCCTTCTGGATATCTT | 507 |
| 1078 | Feline parvovirus | GTGCACCATATTATTCTTTTGAAGC | 939 | ATCCAATCTCCTTCTGGATATCTTC | 513 |
| 1079 | Feline parvovirus | GCTACTATTATGAGACCAGCTGAGG | 506 | TGTTGTCTACCAAATGCATATCTTG | 510 |
| 1080 | Feline parvovirus | GTGCACCATATTATTCTTTTGAAGC | 939 | AATCCAATCTCCTTCTGGATATCTT | 507 |
| 1081 | Feline parvovirus | AATGTACCACCAGTTTATCCAAATG | 500 | TACCTTTCCACCAAAAATCTGAGTA | 501 |
| 1082 | Feline parvovirus | GTGCACCATATTATTCTTTTGAAGC | 939 | TGTAAATCTCTCAGGTGTTTCTCCT | 940 |
| 1083 | Feline parvovirus | GTGCACCATATTATTCTTTTGAAGC | 939 | TAAATCTCTCAGGTGTTTCTCCTGT | 938 |
| 1084 | Feline parvovirus | CTGAAGGAGCTACTAACTTTGGTGA | 941 | TGTTGTCTACCAAATGCATATCTTG | 510 |
| 1085 | Feline parvovirus | ACTATTATGAGACCAGCTGAGGTTG | 511 | CATGTTGTCTACCAAATGCATATCT | 514 |
| 1086 | Feline parvovirus | AGTTCAACAAGATAAAAGACGTGGT | 942 | TGTAAATCTCTCAGGTGTTTCTCCT | 940 |
| 1087 | Feline parvovirus | TAAAAGACGTGGTGTAACTCAAATG | 509 | TGTAAATCTCTCAGGTGTTTCTCCT | 940 |
| 1088 | Feline parvovirus | TAAAAGACGTGGTGTAACTCAAATG | 509 | TAAATCTCTCAGGTGTTTCTCCTGT | 938 |
| 1089 | Feline parvovirus | TAAAAGACGTGGTGTAACTCAAATG | 509 | TGTTGTCTACCAAATGCATATCTTG | 510 |
| 1090 | Feline parvovirus | AATGTACCACCAGTTTATCCAAATG | 500 | TTTCCACCAAAAATCTGAGTAAGTT | 504 |
| 1091 | Feline parvovirus | ATGTATTGCTACCAACAGATCCAAT | 494 | GTTAAATTAGGCGCAACTCTTACAA | 943 |
| 1092 | Feline parvovirus | ATGTATTGCTACCAACAGATCCAAT | 494 | ATATTCATTTGTTAAATTAGGCGCA | 505 |
| 1093 | Feline parvovirus | AGATATCCAGAAGGAGATTGGATTC | 492 | GTTAAATTAGGCGCAACTCTTACAA | 943 |
| 1094 | Feline parvovirus | AATGGTCAAATTTGGGATAAAGAAT | 944 | TACCTTTCCACCAAAAATCTGAATA | 945 |
| 1095 | Feline parvovirus | TTTCTAAATTCTTTGCCTCAATCTG | 946 | TGTTGTCTACCAAATGCATATCTTG | 510 |
| 1096 | Feline parvovirus | GGCTTACCACCATTTCTAAATTCTT | 522 | TGTTGTCTACCAAATGCATATCTTG | 510 |
| 1097 | Feline parvovirus | AATGGTCAAATTTGGGATAAAGAAT | 944 | TACCTTTCCACCAAAAATCTGAGTA | 501 |
| 1098 | Feline parvovirus | TTACCACCATTTCTAAATTCTTTGC | 947 | TGTTGTCTACCAAATGCATATCTTG | 510 |
| 1099 | Feline parvovirus | TGAAGGAGCTACTAACTTTGGTGAT | 948 | TGTTGTCTACCAAATGCATATCTTG | 510 |
| 1100 | Feline parvovirus | ATGTATTGCTACCAACAGATCCAAT | 494 | TCATATTCATTCGTTAAATTAGGCG | 949 |
| 1101 | Giardia spp | GCTAGTAACGGAAAGTGCATTATGT | 950 | ACACCAGTGATTTGGTTGTTATTTC | 951 |
| 1102 | Giardia spp | TGGACAAAGGTATGACTGTTATCTTC | 952 | CAATTACAACGTTCTCCCATAACTT | 953 |
| 1103 | Giardia spp | TCTGGACAAAGGTATGACTGTTATCT | 954 | CAATTACAACGTTCTCCCATAACTT | 953 |
| 1104 | Giardia spp | CTTTGTGCACCTTTCTACAGCTATT | 955 | GCAGAAGATAACAGTCATACCTTTGT | 956 |
| 1105 | Giardia spp | CTTTGTGCACCTTTCTACAGCTATT | 955 | GTGCAGAAGATAACAGTCATACCTTT | 957 |
| 1106 | Giardia spp | CTTTGTGCACCTTTCTACAGCTATT | 955 | GAAGATAACAGTCATACCTTTGTCCA | 958 |
| 1107 | Giardia spp | GCCATGTAATAATAGGACACTCTGAA | 959 | TATTCACCTCCATAGTGTTATTGGC | 960 |
| 1108 | Giardia spp | CTTTGTGCACCTTTCTACAGCTATT | 955 | AGATAACAGTCATACCTTTGTCCAGA | 961 |
| 1109 | Giardia spp | AGCCATGTAATAATAGGACACTCTGA | 962 | TATTCACCTCCATAGTGTTATTGGC | 960 |
| 1110 | Giardia spp | CTGAGCCATGTAATAATAGGACACTC | 963 | TATTCACCTCCATAGTGTTATTGGC | 960 |
| 1111 | Giardia spp | TGAGCCATGTAATAATAGGACACTCT | 964 | TATTCACCTCCATAGTGTTATTGGC | 960 |
| 1112 | Giardia spp | GAGCCATGTAATAATAGGACACTCTG | 965 | TATTCACCTCCATAGTGTTATTGGC | 960 |
| 1113 | Giardia spp | GCTGAGCCATGTAATAATAGGACAC | 966 | ATATTCACCTCCATAGTGTTATTGGC | 967 |
| 1114 | Giardia spp | CTTTGTGCACCTTTCTACAGCTATT | 955 | AAGATAACAGTCATACCTTTGTCCAG | 968 |
| 1115 | Giardia spp | AGGACACTCTGAAAGACGTAGAATC | 969 | ATATTCACCTCCATAGTGTTATTGGC | 967 |
| 1116 | Giardia spp | CTTTGTGCACCTTTCTACAGCTATT | 955 | ATAACAGTCATACCTTTGTCCAGAGC | 970 |
| 1117 | Giardia spp | TAGGACACTCTGAAAGACGTAGAATC | 971 | TATTCACCTCCATAGTGTTATTGGC | 960 |
| 1118 | Giardia spp | AAAATAGCAGCACAGAACGTGTATC | 972 | GAAGATAACAGTCATACCTTTGTCCA | 958 |
| 1119 | Giardia spp | GAAAATAGCAGCACAGAACGTGTAT | 973 | GAAGATAACAGTCATACCTTTGTCCA | 958 |
| 1120 | Giardia spp | AAAATAGCAGCACAGAACGTGTATC | 972 | AGATAACAGTCATACCTTTGTCCAGA | 961 |
| 1121 | Giardia spp | GAAAATAGCAGCACAGAACGTGTAT | 973 | AGATAACAGTCATACCTTTGTCCAGA | 961 |
| 1122 | Giardia spp | GCTGAGCCATGTAATAATAGGACAC | 966 | GAGCAATATTCACCTCCATAGTGTTA | 974 |
| 1123 | Giardia spp | AGGACACTCTGAAAGACGTAGAATC | 969 | GAGCAATATTCACCTCCATAGTGTTA | 974 |
| 1124 | Giardia spp | AAAATAGCAGCACAGAACGTGTATC | 972 | GAGCAATATTCACCTCCATAGTGTTA | 974 |
| 1125 | Giardia spp | GAAAATAGCAGCACAGAACGTGTAT | 973 | GAGCAATATTCACCTCCATAGTGTTA | 974 |
| 1126 | Giardia spp | GGACACTCTGAAAGACGTAGAATCA | 975 | GAGCAATATTCACCTCCATAGTGTTA | 974 |
| 1127 | Giardia spp | GCCATGTAATAATAGGACACTCTGAA | 959 | CAATTACAACGTTCTCCCATAACTT | 953 |
| 1128 | Giardia spp | TGAGCCATGTAATAATAGGACACTCT | 964 | CAATTACAACGTTCTCCCATAACTT | 953 |
| 1129 | Giardia spp | GAGCCATGTAATAATAGGACACTCTG | 965 | CAATTACAACGTTCTCCCATAACTT | 953 |
| 1130 | Giardia spp | CTGAGCCATGTAATAATAGGACACTC | 963 | CAATTACAACGTTCTCCCATAACTT | 953 |
| 1131 | Giardia spp | AGCCATGTAATAATAGGACACTCTGA | 962 | CAATTACAACGTTCTCCCATAACTT | 953 |
| 1132 | Giardia spp | CTTTGTGCACCTTTCTACAGCTATT | 955 | TTCAGAGTGTCCTATTATTACATGGC | 976 |
| 1133 | Giardia spp | GCTGAGCCATGTAATAATAGGACAC | 966 | AATTACAACGTTCTCCCATAACTTCT | 977 |
| 1134 | Giardia spp | GCCATGTAATAATAGGACACTCTGAA | 959 | TACAACGTTCTCCCATAACTTCTTT | 978 |
| 1135 | Giardia spp | GCCATGTAATAATAGGACACTCTGAA | 959 | TTACAACGTTCTCCCATAACTTCTT | 979 |
| 1136 | Giardia spp | CTTTGTGCACCTTTCTACAGCTATT | 955 | TCAGAGTGTCCTATTATTACATGGCT | 980 |
| 1137 | Giardia spp | CTTTGTGCACCTTTCTACAGCTATT | 955 | AGAGTGTCCTATTATTACATGGCTCA | 981 |
| 1138 | Giardia spp | CTTTGTGCACCTTTCTACAGCTATT | 955 | CAGAGTGTCCTATTATTACATGGCTC | 982 |
| 1139 | Giardia spp | CTTTGTGCACCTTTCTACAGCTATT | 955 | GAGTGTCCTATTATTACATGGCTCAG | 983 |
| 1140 | Giardia spp | GCCATGTAATAATAGGACACTCTGAA | 959 | ACGTTCTCCCATAACTTCTTTGATT | 984 |
| 1141 | Giardia spp | GCCATGTAATAATAGGACACTCTGAA | 959 | AACGTTCTCCCATAACTTCTTTGAT | 985 |
| 1142 | Giardia spp | TAGGACACTCTGAAAGACGTAGAATC | 971 | CAATTACAACGTTCTCCCATAACTT | 953 |
| 1143 | Giardia spp | AGGACACTCTGAAAGACGTAGAATC | 969 | AATTACAACGTTCTCCCATAACTTCT | 977 |
| 1144 | Giardia spp | AGCCATGTAATAATAGGACACTCTGA | 962 | TACAACGTTCTCCCATAACTTCTTT | 978 |
| 1145 | Giardia spp | GAGCCATGTAATAATAGGACACTCTG | 965 | TTACAACGTTCTCCCATAACTTCTT | 979 |
| 1146 | Giardia spp | GAGCCATGTAATAATAGGACACTCTG | 965 | TACAACGTTCTCCCATAACTTCTTT | 978 |
| 1147 | Giardia spp | CTGAGCCATGTAATAATAGGACACTC | 963 | TACAACGTTCTCCCATAACTTCTTT | 978 |
| 1148 | Giardia spp | CTGAGCCATGTAATAATAGGACACTC | 963 | TTACAACGTTCTCCCATAACTTCTT | 979 |
| 1149 | Giardia spp | TGAGCCATGTAATAATAGGACACTCT | 964 | TTACAACGTTCTCCCATAACTTCTT | 979 |
| 1150 | Giardia spp | TGAGCCATGTAATAATAGGACACTCT | 964 | TACAACGTTCTCCCATAACTTCTTT | 978 |
| 1151 | Lawsonia intracellularis | TCCCTCATTAATTACTTCATTAGCTC | 986 | TCATTCGCATTCATATTTGTACTTG | 987 |
| 1152 | Lawsonia intracellularis | AAACAATAAACTTGGTCTTCTTTCTGAT | 988 | TCATTCGCATTCATATTTGTACTTG | 987 |
| 1153 | Lawsonia intracellularis | ATCCCTCATTAATTACTTCATTAGCTC | 989 | TTGAATACAATCCACAACAAATTGA | 990 |
| 1154 | Lawsonia intracellularis | CAAACAATAAACTTGGTCTTCTTTCTG | 991 | TCATTCGCATTCATATTTGTACTTG | 987 |
| 1155 | Lawsonia intracellularis | AAACAATAAACTTGGTCTTCTTTCTGA | 992 | TCATTCGCATTCATATTTGTACTTG | 987 |
| 1156 | Lawsonia intracellularis | TTCATTAGCTCAAGTTAAACAAGCTG | 993 | TCATTCGCATTCATATTTGTACTTGT | 994 |
| 1157 | Lawsonia intracellularis | GCAAACAATAAACTTGGTCTTCTTTC | 995 | TCATTCGCATTCATATTTGTACTTG | 987 |
| 1158 | Lawsonia intracellularis | ACTTGCAAACAATAAACTTGGTCTTC | 996 | TCATTCGCATTCATATTTGTACTTG | 987 |
| 1159 | Lawsonia intracellularis | TGCAAACAATAAACTTGGTCTTCTT | 997 | TCATTCGCATTCATATTTGTACTTGT | 994 |
| 1160 | Lawsonia intracellularis | TTGCAAACAATAAACTTGGTCTTCT | 998 | TCATTCGCATTCATATTTGTACTTGT | 994 |
| 1161 | Lawsonia intracellularis | CTTGCAAACAATAAACTTGGTCTTC | 999 | TCATTCGCATTCATATTTGTACTTGT | 994 |
| 1162 | Lawsonia intracellularis | ACTTGCAAACAATAAACTTGGTCTT | 1000 | TCATTCGCATTCATATTTGTACTTGT | 994 |
| 1163 | Lawsonia intracellularis | CTTGCAAACAATAAACTTGGTCTTCT | 1001 | TCATTCGCATTCATATTTGTACTTG | 987 |
| 1164 | Lawsonia intracellularis | GCAAACAATAAACTTGGTCTTCTTT | 1002 | TCATTCGCATTCATATTTGTACTTGT | 994 |
| 1165 | Lawsonia intracellularis | TGCAAACAATAAACTTGGTCTTCTTT | 1003 | TCATTCGCATTCATATTTGTACTTG | 987 |
| 1166 | Lawsonia intracellularis | TTGCAAACAATAAACTTGGTCTTCTT | 1004 | TCATTCGCATTCATATTTGTACTTG | 987 |
| 1167 | Lawsonia intracellularis | CACTTGCAAACAATAAACTTGGTCT | 1005 | TCATTCGCATTCATATTTGTACTTGT | 994 |
| 1168 | Lawsonia intracellularis | AAACAATAAACTTGGTCTTCTTTCTG | 1006 | TCATTCGCATTCATATTTGTACTTG | 987 |
| 1169 | Lawsonia intracellularis | CAAACAATAAACTTGGTCTTCTTTCT | 1007 | TCATTCGCATTCATATTTGTACTTG | 987 |
| 1170 | Lawsonia intracellularis | CACTTGCAAACAATAAACTTGGTCTT | 1008 | TCATTCGCATTCATATTTGTACTTG | 987 |
| 1171 | Lawsonia intracellularis | TCATTAGCTCAAGTTAAACAAGCTG | 1009 | TCATTCGCATTCATATTTGTACTTGT | 994 |
| 1172 | Lawsonia intracellularis | TCCCTCATTAATTACTTCATTAGCTC | 986 | TTAGCATTCATATTTGTACTTGTCCC | 1010 |
| 1173 | Lawsonia intracellularis | TTGCAAACAATAAACTTGGTCTTCT | 998 | TCATTCGCATTCATATTTGTACTTG | 987 |
| 1174 | Lawsonia intracellularis | CTTGCAAACAATAAACTTGGTCTTC | 999 | TCATTCGCATTCATATTTGTACTTG | 987 |
| 1175 | Lawsonia intracellularis | TGCAAACAATAAACTTGGTCTTCTT | 997 | TCATTCGCATTCATATTTGTACTTG | 987 |
| 1176 | Lawsonia intracellularis | ACTTGCAAACAATAAACTTGGTCTT | 1000 | TCATTCGCATTCATATTTGTACTTG | 987 |
| 1177 | Lawsonia intracellularis | GCAAACAATAAACTTGGTCTTCTTT | 1002 | TCATTCGCATTCATATTTGTACTTG | 987 |
| 1178 | Lawsonia intracellularis | CACTTGCAAACAATAAACTTGGTCT | 1005 | TCATTCGCATTCATATTTGTACTTG | 987 |
| 1179 | Lawsonia intracellularis | CACTTGCAAACAATAAACTTGGTC | 1011 | TCATTCGCATTCATATTTGTACTTGT | 994 |
| 1180 | Lawsonia intracellularis | TTGCAAACAATAAACTTGGTCTTC | 1012 | TCATTCGCATTCATATTTGTACTTGT | 994 |
| 1181 | Lawsonia intracellularis | TCCCTCATTAATTACTTCATTAGCTC | 986 | CATTCATATTTGTACTTGTCCCTGC | 1013 |
| 1182 | Lawsonia intracellularis | TCCCTCATTAATTACTTCATTAGCTC | 986 | GCATTCATATTTGTACTTGTCCCTG | 1014 |
| 1183 | Lawsonia intracellularis | TCCCTCATTAATTACTTCATTAGCTC | 986 | TTGAATACAATCCACAACAAATTGA | 990 |
| 1184 | Lawsonia intracellularis | TCCCTCATTAATTACTTCATTAGCTC | 986 | TCCTTGAATACAATCCACAACAAAT | 1015 |
| 1185 | Lawsonia intracellularis | TCCCTCATTAATTACTTCATTAGCTC | 986 | AGCATTCATATTTGTACTTGTCCCT | 1016 |
| 1186 | Lawsonia intracellularis | TCCCTCATTAATTACTTCATTAGCTC | 986 | CATTCGCATTCATATTTGTACTTGT | 1017 |
| 1187 | Lawsonia intracellularis | CACTTGCAAACAATAAACTTGGTC | 1011 | TCATTCGCATTCATATTTGTACTTG | 987 |
| 1188 | Lawsonia intracellularis | TTGCAAACAATAAACTTGGTCTTC | 1012 | TCATTCGCATTCATATTTGTACTTG | 987 |
| 1189 | Lawsonia intracellularis | TGCAAACAATAAACTTGGTCTTCT | 1018 | TCATTCGCATTCATATTTGTACTTG | 987 |
| 1190 | Lawsonia intracellularis | CAGCACTTGCAAACAATAAACTTG | 1019 | TCATTCGCATTCATATTTGTACTTG | 987 |
| 1191 | Lawsonia intracellularis | CTTGCAAACAATAAACTTGGTCTT | 1020 | TCATTCGCATTCATATTTGTACTTG | 987 |
| 1192 | Lawsonia intracellularis | GCACTTGCAAACAATAAACTTGGT | 1021 | TCATTCGCATTCATATTTGTACTTG | 987 |
| 1193 | Lawsonia intracellularis | ACTTGCAAACAATAAACTTGGTCT | 1022 | TCATTCGCATTCATATTTGTACTTG | 987 |
| 1194 | Lawsonia intracellularis | AGCACTTGCAAACAATAAACTTGG | 1023 | TCATTCGCATTCATATTTGTACTTG | 987 |
| 1195 | Lawsonia intracellularis | GCAGCACTTGCAAACAATAAACTT | 1024 | TCATTCGCATTCATATTTGTACTTG | 987 |
| 1196 | Lawsonia intracellularis | GCAAACAATAAACTTGGTCTTCTT | 1025 | TCATTCGCATTCATATTTGTACTTG | 987 |
| 1197 | Lawsonia intracellularis | TCCCTCATTAATTACTTCATTAGCTC | 986 | ATATTTGTACTTGTCCCTGCACCT | 1026 |
| 1198 | Lawsonia intracellularis | TCCCTCATTAATTACTTCATTAGCTC | 986 | CTCCTTGAATACAATCCACAACAA | 1027 |
| 1199 | Lawsonia intracellularis | GCAGCACTTGCAAACAATAAACT | 1028 | TCATTCGCATTCATATTTGTACTTG | 987 |
| 1200 | Lawsonia intracellularis | GCACTTGCAAACAATAAACTTGG | 1029 | TCATTCGCATTCATATTTGTACTTG | 987 |
| 1201 | Mesocestoides spp | ACTCTGGATAATTGTTACAGATCGC | 1030 | TTTAGAGTCCACTCATTCCGATTAC | 1031 |
| 1202 | Mesocestoides spp | CTTCTGGTGACTCTGGATAATTGTT | 1032 | TTTAGAGTCCACTCATTCCGATTAC | 1031 |
| 1203 | Mesocestoides spp | AAATGTTGTGGAGTGCTGTTGAT | 1033 | ATTGATATGCTTAAGTTAGCGG | 1034 |
| 1204 | Mesocestoides spp | CTTATAAACTATCACTGCGCGTAAAAAG | 1035 | ATCAACAGCACTCCACAACATTT | 1036 |
| 1205 | Mesocestoides spp | TTATAAACTATCACTGCGCGTAAAAAG | 1037 | ATCAACAGCACTCCACAACATTT | 1036 |
| 1206 | Mesocestoides spp | CTTATAAACTATCACTGCGCGTAAAAA | 1038 | ATCAACAGCACTCCACAACATTT | 1036 |
| 1207 | Mesocestoides spp | TATAAACTATCACTGCGCGTAAAAAG | 1039 | ATCAACAGCACTCCACAACATTT | 1036 |
| 1208 | Mesocestoides spp | GCTTATAAACTATCACTGCGCGTAAA | 1040 | ATCAACAGCACTCCACAACATTT | 1036 |
| 1209 | Mesocestoides spp | TTATAAACTATCACTGCGCGTAAAAA | 1041 | ATCAACAGCACTCCACAACATTT | 1036 |
| 1210 | Mesocestoides spp | AAATGTTGTGGAGTGCTGTTGAT | 1033 | GATATGCTTAAGTTCAGCGGGTACT | 1042 |
| 1211 | Mesocestoides spp | TAAGGCTTCTTCTTAATGTGGCTTAGA | 1043 | ATCAACAGCACTCCACAACATTT | 1036 |
| 1212 | Mesocestoides spp | ATAAACTATCACTGCGCGTAAAAAG | 1044 | ATCAACAGCACTCCACAACATTT | 1036 |
| 1213 | Mesocestoides spp | CTTATAAACTATCACTGCGCGTAAA | 1045 | ATCAACAGCACTCCACAACATTT | 1036 |
| 1214 | Mesocestoides spp | TATAAACTATCACTGCGCGTAAAAA | 1046 | ATCAACAGCACTCCACAACATTT | 1036 |
| 1215 | Mesocestoides spp | GAATAATGGAATAGGACTTCGGTTCT | 1047 | CATCGTTTATGGTCAGAACTAGGAC | 1048 |
| 1216 | Mesocestoides spp | ACGTTTACTTTGAACAAATTTGAGTG | 1049 | GTTTATGGTCAGAACTAGGACGGTA | 1050 |
| 1217 | Mesocestoides spp | ACGTTTACTTTGAACAAATTTGAGTG | 1049 | TTATGGTCAGAACTAGGACGGTATC | 1051 |
| 1218 | Mesocestoides spp | ACGTTTACTTTGAACAAATTTGAGTG | 1049 | CATCGTTTATGGTCAGAACTAGGAC | 1048 |
| 1219 | Mesocestoides spp | ACGTTTACTTTGAACAAATTTGAGTG | 1049 | TATGGTCAGAACTAGGACGGTATCT | 1052 |
| 1220 | Mesocestoides spp | ACGTTTACTTTGAACAAATTTGAGTG | 1049 | TTTATGGTCAGAACTAGGACGGTAT | 1053 |
| 1221 | Mesocestoides spp | GTAGCTATCGTTGTTGTCGTTAGTG | 1054 | ACTCAAATTTGTTCAAAGTAAACGTG | 1055 |
| 1222 | Mesocestoides spp | CGTAGCTATCGTTGTTGTCGTTAGT | 1056 | ACTCAAATTTGTTCAAAGTAAACGTG | 1055 |
| 1223 | Mesocestoides spp | GTTTACTTTGAACAAATTTGAGTGCT | 1057 | CATCGTTTATGGTCAGAACTAGGAC | 1048 |
| 1224 | Mesocestoides spp | AAGTTTTGCATGGAATAATGGAATAG | 1058 | CATCGTTTATGGTCAGAACTAGGAC | 1048 |
| 1225 | Mesocestoides spp | GTAGCTATCGTCGTTGTCGTTAGTG | 1059 | ACTCAAATTTGTTCAAAGTAAACGTG | 1055 |
| 1226 | Mesocestoides spp | ATGGCTTCTTCTTAATGTGGCTTAG | 1060 | ATCAACAGCACTCCACAACATTT | 1036 |
| 1227 | Mesocestoides spp | AAATGTTGTGGAGTGCTGTTGAT | 1033 | ATATGCTTAAGTTCAGCGGGTACT | 1061 |
| 1228 | Mesocestoides spp | AAAGTTTTGCATGGAATAATGGAATA | 1062 | CATCGTTTATGGTCAGAACTAGGAC | 1048 |
| 1229 | Mesocestoides spp | GATGGCTTCTTCTTAATGTGGCTTA | 1063 | ATCAACAGCACTCCACAACATTT | 1036 |
| 1230 | Mesocestoides spp | AAGGCTTCTTCTTAATGTGGCTTAG | 1064 | ATCAACAGCACTCCACAAGATTT | 1036 |
| 1231 | Mesocestoides spp | TAAGGCTTCTTCTTAATGTGGCTTAG | 1065 | ATCAACAGCACTCCACAACATTT | 1036 |
| 1232 | Mesocestoides spp | TAAACTATCACTGCGCGTAAAAAG | 1066 | ATCAACAGCACTCCACAACATTT | 1036 |
| 1233 | Mesocestoides spp | CTAAGGCTTCTTAATGTGGCTTA | 1067 | ATCAACAGCACTCCACAACATTT | 1036 |
| 1234 | Mesocestoides spp | GAGCGTCGGCTTATAAACTATCAC | 1068 | ATCAACAGCACTCCACAACATTT | 1036 |
| 1235 | Mesocestoides spp | GTCGGCTTATAAACTATCACTGCG | 1069 | ATCAACAGCACTCCACAACATTT | 1036 |
| 1236 | Mesocestoides spp | CGTCGGCTTATAAACTATCACTGC | 1070 | ATCAACAGCACTCCACAACATTT | 1036 |
| 1237 | Mesocestoides spp | GCGTCGGCTTATAAACTATCACTG | 1071 | ATCAACAGCACTCCACAACATTT | 1036 |
| 1238 | Mesocestoides spp | TTATAAACTATCACTGCGCGTAAA | 1072 | ATCAACAGCACTCCACAACATTT | 1036 |
| 1239 | Mesocestoides spp | ATAAACTATCACTGCGCGTAAAAA | 1073 | ATCAACAGCACTCCACAACATTT | 1036 |
| 1240 | Mesocestoides spp | AATAATGGAATAGGACTTCGGTTCT | 1074 | GTTTATGGTCAGAACTAGGACGGTA | 1050 |
| 1241 | Mesocestoides spp | AATAATGGAATAGGACTTCGGTTCT | 1074 | TTATGGTCAGAACTAGGACGGTATC | 1051 |
| 1242 | Mesocestoides spp | GAATAATGGAATAGGACTTCGGTTC | 1075 | GTTTATGGTCAGAACTAGGACGGTA | 1050 |
| 1243 | Mesocestoides spp | GAATAATGGAATAGGACTTCGGTTC | 1075 | TTATGGTCAGAACTAGGACGGTATC | 1051 |
| 1244 | Mesocestoides spp | AATAATGGAATAGGACTTCGGTTCT | 1074 | CATCGTTTATGGTCAGAACTAGGAC | 1048 |
| 1245 | Mesocestoides spp | AATAGGACTTCGGTTCTATTTCGTT | 1076 | TATGGTCAGAACTAGGACGGTATCT | 1052 |
| 1246 | Mesocestoides spp | GAATAATGGAATAGGACTTCGGTTC | 1075 | CATCGTTTATGGTCAGAACTAGGAC | 1048 |
| 1247 | Mesocestoides spp | AATAGGACTTCGGTTCTATTTCGTT | 1076 | TTTATGGTCAGAACTAGGACGGTAT | 1053 |
| 1248 | Mesocestoides spp | AATAATGGAATAGGACTTCGGTTCT | 1074 | TATGGTCAGAACTAGGACGGTATCT | 1052 |
| 1249 | Mesocestoides spp | AATAATGGAATAGGACTTCGGTTCT | 1074 | TTTATGGTCAGAACTAGGACGGTAT | 1053 |
| 1250 | Mesocestoides spp | GAATAATGGAATAGGACTTCGGTTC | 1075 | TATGGTCAGAACTAGGACGGTATCT | 1052 |
| 1251 | Neorickettsia risticii | ACAAGTTGGTTCAATTTCTGCTAAC | 1077 | AGAAGGACATAAGGATTGTCAAACTC | 1078 |
| 1252 | Neorickettsia risticii | ACAAGTTGGTTCAATTTCTGCTAAC | 1077 | GATTGTCAAACTCAACAATCATTTTC | 1079 |
| 1253 | Neorickettsia risticii | ACAAGTTGGTTCAATTTCTGCTAAC | 1077 | GAGAAGGACATAAGGATTGTCAAACT | 1080 |
| 1254 | Neorickettsia risticii | ACAAGTTGGTTCAATTTCTGCTAAC | 1077 | TTTCTCCGGGTTTGTTATAAAGTAAG | 1081 |
| 1255 | Neorickettsia risticii | ATCTATAGCTGGTCTGAGAGGATGAT | 1082 | CACTAAAAGAGCTTTACAACCCAAG | 1083 |
| 1256 | Neorickettsia risticii | ATCTATAGCTGGTCTGAGAGGATGAT | 1082 | ACTAAAAGAGCTTTACAACCCAAGG | 1084 |
| 1257 | Neorickettsia risticii | ATCTATAGCTGGTCTGAGAGGATGAT | 1082 | TTCCCCACTAAAAGAGCTTTACAAC | 1085 |
| 1258 | Neorickettsia risticii | TAGTAGGGAATAACCAGTGGAAACAT | 1086 | ATCTTCCCCACTAAAAGAGGTTTAC | 1087 |
| 1259 | Neorickettsia risticii | GTAGGGAATAACCAGTGGAAACATT | 1088 | TATCTTCCCCACTAAAAGAGCTTTAC | 1089 |
| 1260 | Neorickettsia risticii | TAGTAGGGAATAACCAGTGGAAACA | 1090 | TATCTTCCCCACTAAAAGAGCTTTAC | 1089 |
| 1261 | Neorickettsia risticii | TAGTAGGGAATAACCAGTGGAAACAT | 1086 | CACTAAAAGAGCTTTACAACCCAAG | 1083 |
| 1262 | Neorickettsia risticii | AGTAGGGAATAACCAGTGGAAACAT | 1091 | TATCTTCCCCACTAAAAGAGCTTTAC | 1089 |
| 1263 | Neorickettsia risticii | TAGTAGGGAATAACCAGTGGAAACAT | 1086 | ACTAAAAGAGCTTTACAACCCAAGG | 1084 |
| 1264 | Neorickettsia risticii | GTAGTAGGGAATAACCAGTGGAAACA | 1092 | ATCTTCCCCACTAAAAGAGCTTTAC | 1087 |
| 1265 | Neorickettsia risticii | GTAGTAGGGAATAACCAGTGGAAACA | 1092 | CACTAAAAGAGCTTTACAACCCAAG | 1083 |
| 1266 | Neorickettsia risticii | GTAGTAGGGAATAACCAGTGGAAACA | 1092 | ACTAAAAGAGCTTTACAACCCAAGG | 1084 |
| 1267 | Neorickettsia risticii | TTAGATTAGCTAGTTGGTGGGGTAAT | 1093 | CACTAAAAGAGCTTTACAACCCAAG | 1083 |
| 1268 | Neorickettsia risticii | GTTAGATTAGCTAGTTGGTGGGGTAA | 1094 | ACTAAAAGAGCTTTACAACCCAAGG | 1084 |
| 1269 | Neorickettsia risticii | TTAGATTAGCTAGTTGGTGGGGTAAT | 1093 | ACTAAAAGAGCTTTACAACCCAAGG | 1084 |
| 1270 | Neorickettsia risticii | TAGATTAGCTAGTTGGTGGGGTAATG | 1095 | ACTAAAAGAGCTTTACAACCCAAGG | 1084 |
| 1271 | Neorickettsia risticii | ACAAGTTGGTTCAATTTCTGCTAAC | 1077 | AGAAGGACATAAGGATTGTCAAACT | 1096 |
| 1272 | Neorickettsia risticii | ACAAGTTGGTTCAATTTCTGCTAAC | 1077 | GGACATAAGGATTGTCAAACTCAAC | 1097 |
| 1273 | Neorickettsia risticii | ACAAGTTGGTTCAATTTCTGCTAAC | 1077 | AGGACATAAGGATTGTCAAACTCAA | 1098 |
| 1274 | Neorickettsia risticii | ACAAGTTGGTTCAATTTCTGCTAAC | 1077 | AAGGACATAAGGATTGTCAAACTCA | 1099 |
| 1275 | Neorickettsia risticii | ACAAGTTGGTTCAATTTCTGCTAAC | 1077 | GGATTGTCAAACTCAACAATCATTT | 1100 |
| 1276 | Neorickettsia risticii | ACAAGTTGGTTCAATTTCTGCTAAC | 1077 | GACATAAGGATTGTCAAACTCAACA | 1101 |
| 1277 | Neorickettsia risticii | ACAAGTTGGTTCAATTTCTGCTAAC | 1077 | GAAGGACATAAGGATTGTCAAACTC | 1102 |
| 1278 | Neorickettsia risticii | ACAAGTTGGTTCAATTTCTGCTAAC | 1077 | GTCAAACTCAACAATCATTTTCTCC | 1103 |
| 1279 | Neorickettsia risticii | ACAAGTTGGTTCAATTTCTGCTAAC | 1077 | TGTCAAACTCAACAATCATTTTCTC | 1104 |
| 1280 | Neorickettsia risticii | ACAAGTTGGTTCAATTTCTGCTAAC | 1077 | TTTCTCCGGGTTTGTTATAAAGTAA | 1105 |
| 1281 | Neorickettsia risticii | ACAAGTTGGTTCAATTTCTGCTAAC | 1077 | TTGTCAAACTCAACAATCATTTTCT | 1106 |
| 1282 | Neorickettsia risticii | ACAAGTTGGTTCAATTTCTGCTAAC | 1077 | TCTCCGGGTTTGTTATAAAGTAAGG | 1107 |
| 1283 | Neorickettsia risticii | ACAAGTTGGTTCAATTTCTGCTAAC | 1077 | CTCCGGGTTTGTTATAAAGTAAGGA | 1108 |
| 1284 | Neorickettsia risticii | ACAAGTTGGTTCAATTTCTGCTAAC | 1077 | TTCTCCGGGTTTGTTATAAAGTAAG | 1109 |
| 1285 | Neorickettsia risticii | ACAAGTTGGTTCAATTTCTGCTAAC | 1077 | CATTTTCTCCGGGTTTGTTATAAAG | 1110 |
| 1286 | Neorickettsia risticii | ACAAGTTGGTTCAATTTCTGCTAAC | 1077 | ATTTTCTCCGGGTTTGTTATAAAGT | 1111 |
| 1287 | Neorickettsia risticii | ACAAGTTGGTTCAATTTCTGCTAAC | 1077 | TTTTCTCCGGGTTTGTTATAAAGTA | 1112 |
| 1288 | Neorickettsia risticii | GATCTATAGCTGGTCTGAGAGGATG | 1113 | TCATTATCTTCCCCACTAAAAGAGC | 1114 |
| 1289 | Neorickettsia risticii | GATCTATAGCTGGTCTGAGAGGATG | 1113 | GTCATTATCTTCCCCACTAAAAGAG | 1115 |
| 1290 | Neorickettsia risticii | ATCTATAGCTGGTCTGAGAGGATGA | 1116 | GTCATTATCTTCCCCACTAAAAGAG | 1115 |
| 1291 | Neorickettsia risticii | GATCTATAGCTGGTCTGAGAGGATG | 1113 | GTACCGTCATTATCTTCCCCACTAA | 1117 |
| 1292 | Neorickettsia risticii | ATCTATAGCTGGTCTGAGAGGATGA | 1116 | GTACCGTCATTATCTTCCCCACTAA | 1117 |
| 1293 | Neorickettsia risticii | CGATCTATAGCTGGTCTGAGAGGAT | 1118 | ATCTTCCCCACTAAAAGAGCTTTAC | 1087 |
| 1294 | Neorickettsia risticii | GATCTATAGCTGGTCTGAGAGGATG | 1113 | CTTCTTCTGTGGGTACCGTCATTAT | 1119 |
| 1295 | Neorickettsia risticii | ATCTATAGCTGGTCTGAGAGGATGA | 1116 | CTTCTTCTGTGGGTACCGTCATTAT | 1119 |
| 1296 | Neorickettsia risticii | GATCTATAGCTGGTCTGAGAGGATG | 1113 | CTTCTGTGGGTACCGTCATTATCTT | 1120 |
| 1297 | Neorickettsia risticii | TCTATAGCTGGTCTGAGAGGATGAT | 1121 | CTTCTTCTGTGGGTACCGTCATTAT | 1119 |
| 1298 | Neorickettsia risticii | GATCTATAGCTGGTCTGAGAGGATG | 1113 | CGTCATTATCTTCCCCACTAAAAGA | 1122 |
| 1299 | Neorickettsia risticii | TCTATAGCTGGTCTGAGAGGATGAT | 1121 | CGTCATTATCTTCCCCACTAAAAGA | 1122 |
| 1300 | Neorickettsia risticii | ATCTATAGCTGGTCTGAGAGGATGAT | 1082 | CGTCATTATCTTCCCCACTAAAAG | 1123 |
| 1301 | Neospora caninum | TTGTTTGTGGCTATAGTGTGTGAAC | 1124 | GAACACTGAACTCTGGATAAGTATCATT | 1125 |
| 1302 | Neospora caninum | TTGTTTGTGGCTATAGTGTGTGAAC | 1124 | CACTGAACTCTGGATAAGTATCATTGAC | 1126 |
| 1303 | Neospora caninum | TTGTTTGTGGCTATAGTGTGTGAAC | 1124 | ACACAGAACACTGAACTCTGGATAAGTA | 1127 |
| 1304 | Neospora caninum | TTGTTTGTGGCTATAGTGTGTGAAC | 1124 | AGAACACTGAACTCTGGATAAGTATCAT | 1128 |
| 1305 | Neospora caninum | TTTGTTTGTGGCTATAGTGTGTGAA | 1129 | GAACACTGAACTCTGGATAAGTATCATT | 1125 |
| 1306 | Neospora caninum | TTGTTTGTGGCTATAGTGTGTGAAC | 1124 | CACAGAACACTGAACTCTGGATAAGTA | 1130 |
| 1307 | Neospora caninum | TTGTTTGTGGCTATAGTGTGTGAAC | 1124 | ACACAGAACACTGAACTCTGGATAAGT | 1131 |
| 1308 | Neospora caninum | TTGTTTGTGGCTATAGTGTGTGAAC | 1124 | AACACAGAACACTGAACTCTGGATAAG | 1132 |
| 1309 | Neospora caninum | TTGTTTGTGGCTATAGTGTGTGAAC | 1124 | ACTGAACTCTGGATAAGTATCATTGAC | 1133 |
| 1310 | Neospora caninum | TTGTTTGTGGCTATAGTGTGTGAAC | 1124 | GAACACTGAACTCTGGATAAGTATCAT | 1134 |
| 1311 | Neospora caninum | TTTGTTTGTGGCTATAGTGTGTGAAC | 1135 | ACACAGAACACTGAACTCTGGATAAG | 1136 |
| 1312 | Neospora caninum | TTTGTTTGTGGCTATAGTGTGTGAAC | 1135 | CACAGAACACTGAACTCTGGATAAGT | 1137 |
| 1313 | Neospora caninum | TTTGTTTGTGGCTATAGTGTGTGAAC | 1135 | AACACAGAACACTGAACTCTGGATAA | 1138 |
| 1314 | Neospora caninum | TTTGTTTGTGGCTATAGTGTGTGAA | 1129 | CACAGAACACTGAACTCTGGATAAGTA | 1130 |
| 1315 | Neospora caninum | TTTGTTTGTGGCTATAGTGTGTGAA | 1129 | ACACAGAACACTGAACTCTGGATAAGT | 1131 |
| 1316 | Neospora caninum | TTTGTTTGTGGCTATAGTGTGTGAA | 1129 | AACACAGAACACTGAACTCTGGATAAG | 1132 |
| 1317 | Neospora caninum | GACTATAGTGTGTGAACGGGTGAAC | 1139 | CACAGAACACTGAACTCTCGATAAGTA | 1140 |
| 1318 | Neospora caninum | GTTTGTGACTATAGTGTGTGAACGG | 1141 | CACAGAACACTGAACTCTCGATAAGTA | 1140 |
| 1319 | Neospora caninum | TGTTTGTGACTATAGTGTGTGAACG | 1142 | CACAGAACACTGAACTCTCGATAAGTA | 1140 |
| 1320 | Neospora caninum | GTTTGTGACTATAGTGTGTGAACGG | 1141 | AACACAGAACACTGAACTCTCGATAAG | 1143 |
| 1321 | Neospora caninum | CTTTTTGTTTGTGACTATAGTGTGTGAA | 1144 | CACAGAACACTGAACTCTCGATAAG | 1145 |
| 1322 | Neospora caninum | TTTTGTTTGTGGCTATAGTGTGTGAA | 1146 | ACACAGAACACTGAACTCTGGATAAG | 1136 |
| 1323 | Neospora caninum | TTTTGTTTGTGGCTATAGTGTGTGAA | 1146 | CACAGAACACTGAACTCTGGATAAGT | 1137 |
| 1324 | Neospora caninum | TTGTTTGTGACTATAGTGTGTGAACG | 1147 | ACACAGAACACTGAACTCTCGATAAG | 1148 |
| 1325 | Neospora caninum | TTGTTTGTGACTATAGTGTGTGAACG | 1147 | AACACAGAACACTGAACTCTCGATAA | 1149 |
| 1326 | Neospora caninum | GTTTGTGGCTATAGTGTGTGAACG | 1150 | CACAGAACACTGAACTCTGGATAAGTA | 1130 |
| 1327 | Neospora caninum | TTGTTTGTGGCTATAGTGTGTGAAC | 1124 | ACACAGAACACTGAACTCTGGATAAG | 1136 |
| 1328 | Neospora caninum | TTGTTTGTGGCTATAGTGTGTGAAC | 1124 | CACAGAACACTGAACTCTGGATAAGT | 1137 |
| 1329 | Neospora caninum | TTGTTTGTGGCTATAGTGTGTGAAC | 1124 | AACACAGAACACTGAACTCTGGATAA | 1138 |
| 1330 | Neospora caninum | TTGTTTGTGGCTATAGTGTGTGAAC | 1124 | CAACACAGAACACTGAACTCTGGATA | 1151 |
| 1331 | Neospora caninum | TGTTTGTGGCTATAGTGTGTGAAC | 1152 | CACAGAACACTGAACTCTGGATAAGTA | 1130 |
| 1332 | Neospora caninum | TTTTGTTTGTGGCTATAGTGTGTGA | 1153 | CACAGAACACTGAACTCTGGATAAGTA | 1130 |
| 1333 | Neospora caninum | TTTGTTTGTGGCTATAGTGTGTGAAC | 1135 | CACAGAACACTGAACTCTGGATAAG | 1154 |
| 1334 | Neospora caninum | TTTGTTTGTGGCTATAGTGTGTGAAC | 1135 | ACACAGAACACTGAACTCTGGATAA | 1155 |
| 1335 | Neospora caninum | TTTGTTTGTGGCTATAGTGTGTGAAC | 1135 | AACACAGAACACTGAACTCTGGATA | 1156 |
| 1336 | Neospora caninum | TTTTGTTTGTGGCTATAGTGTGTGA | 1153 | ACACAGAACACTGAACTCTGGATAAGT | 1131 |
| 1337 | Neospora caninum | TTTTGTTTGTGGCTATAGTGTGTGA | 1153 | AACACAGAACACTGAACTCTGGATAAG | 1132 |
| 1338 | Neospora caninum | TTTGTTTGTGGCTATAGTGTGTGAAC | 1135 | CAACACAGAACACTGAACTCTGGAT | 1157 |
| 1339 | Neospora caninum | TTTTTGTTTGTGGCTATAGTGTGTG | 1158 | GAACACTGAACTCTGGATAAGTATCATT | 1125 |
| 1340 | Neospora caninum | TTTTTGTTTGTGGCTATAGTGTGTG | 1158 | CACTGAACTCTGGATAAGTATCATTGAC | 1126 |
| 1341 | Neospora caninum | TTTTTGTTTGTGGCTATAGTGTGTG | 1158 | ACACAGAACACTGAAGTGTGGATAAGTA | 1127 |
| 1342 | Neospora caninum | TTTTTGTTTGTGGCTATAGTGTGTG | 1158 | AGAACACTGAACTCTGGATAAGTATCAT | 1128 |
| 1343 | Neospora caninum | TTGTTTGTGGCTATAGTGTGTGAA | 1159 | CACAGAACACTGAACTCTGGATAAGTA | 1130 |
| 1344 | Neospora caninum | TTGTTTGTGGCTATAGTGTGTGAA | 1159 | ACACAGAACACTGAACTCTGGATAAGT | 1131 |
| 1345 | Neospora caninum | TTTGTTTGTGGCTATAGTGTGTGAA | 1129 | CACAGAACACTGAACTCTGGATAAGT | 1137 |
| 1346 | Neospora caninum | TTTGTTTGTGGCTATAGTGTGTGAA | 1129 | ACACAGAACACTGAACTCTGGATAAG | 1136 |
| 1347 | Neospora caninum | TTTGTTTGTGGCTATAGTGTGTGAA | 1129 | AACACAGAACACTGAACTCTGGATAA | 1138 |
| 1348 | Neospora caninum | TTTGTTTGTGGCTATAGTGTGTGAA | 1129 | CAACACAGAACACTGAACTCTGGATA | 1151 |
| 1349 | Neospora caninum | TTTTTGTTTGTGGCTATAGTGTGTGA | 1160 | ACACAGAACACTGAACTCTGGATAAG | 1136 |
| 1350 | Neospora caninum | TTTTTGTTTGTGGCTATAGTGTGTGA | 1160 | CACAGAACACTGAACTCTGGATAAGT | 1137 |
| 1351 | Strongyloides spp | TGAGGTAATTCTTGAGCTAATACACG | 1161 | AATACCCTACCATCGAAAGTTGATAA | 1162 |
| 1352 | Strongyloides spp | AGGTAATTCTTGAGCTAATACACGCT | 1163 | AATACCCTACCATCGAAAGTTGATAA | 1162 |
| 1353 | Strongyloides spp | TGAGGTAATTCTTGAGCTAATACACG | 1161 | CATCGAAAGTTGATAAACCAGATACA | 1164 |
| 1354 | Strongyloides spp | AGGTAATTCTTGAGCTAATACACGCT | 1163 | CATCGAAAGTTGATAAACCAGATACA | 1164 |
| 1355 | Strongyloides spp | TGAGGTAATTCTTGAGCTAATACACG | 1161 | CGAAAGTTGATAAACCAGATACACAT | 1165 |
| 1356 | Strongyloides spp | CTTGAGCTAATACACGCTTTTTATACC | 1166 | AATACCCTACCATCGAAAGTTGATA | 1167 |
| 1357 | Strongyloides spp | CTTGAGCTAATACACGCTTTTTATACC | 1166 | ATACCCTACCATCGAAAGTTGATAA | 1168 |
| 1358 | Strongyloides spp | TAATTCTTGAGCTAATACACGCTTTTT | 1169 | CTACCATCGAAAGTTGATAAACCAG | 1170 |
| 1359 | Strongyloides spp | GTAATTCTTGAGCTAATACACGCTTT | 1171 | CTACCATCGAAAGTTGATAAACCAG | 1170 |
| 1360 | Strongyloides spp | GGTAATTCTTGAGCTAATACACGCTT | 1172 | AATACCCTACCATCGAAAGTTGATAA | 1162 |
| 1361 | Strongyloides spp | TAATTCTTGAGCTAATACACGCTTTTT | 1169 | AATACCCTACCATCGAAAGTTGATA | 1167 |
| 1362 | Strongyloides spp | TAATTCTTGAGCTAATACACGCTTTTT | 1169 | ATACCCTACCATCGAAAGTTGATAA | 1168 |
| 1363 | Strongyloides spp | GTAATTCTTGAGCTAATACACGCTTTT | 1171 | ATACCCTACCATCGAAAGTTGATAA | 1168 |
| 1364 | Strongyloides spp | GTAATTCTTGAGCTAATACACGCTTTT | 1171 | AATACCCTACCATCGAAAGTTGATA | 1167 |
| 1365 | Strongyloides spp | TTGAGCTAATACACGCTTTTTATACC | 1173 | AATACCCTACCATCGAAAGTTGATAA | 1162 |
| 1366 | Strongyloides spp | TGGATAACTGAGGTAATTCTTGAGC | 1174 | AGTTGATAAACCAGATACACATACGGT | 1175 |
| 1367 | Strongyloides spp | AATTCTTGAGCTAATACACGCTTTTT | 1176 | AATACCCTACCATCGAAAGTTGATAA | 1162 |
| 1368 | Strongyloides spp | TGGATAACTGAGGTAATTCTTGAGC | 1174 | CGAAAGTTGATAAACCAGATACACATA | 1177 |
| 1369 | Strongyloides spp | TGGATAACTGAGGTAATTCTTGAGC | 1174 | AAGTTGATAAACCAGATACACATACGG | 1178 |
| 1370 | Strongyloides spp | TGAGGTAATTCTTGAGCTAATACACG | 1161 | CTACCATCGAAAGTTGATAAACCAG | 1170 |
| 1371 | Strongyloides spp | AGGTAATTCTTGAGCTAATACACGCT | 1163 | CTACCATCGAAAGTTGATAAACCAG | 1170 |
| 1372 | Strongyloides spp | TGAGGTAATTCTTGAGCTAATACACG | 1161 | AATACCCTACCATCGAAAGTTGATA | 1167 |
| 1373 | Strongyloides spp | TGAGGTAATTCTTGAGCTAATACACG | 1161 | ATACCCTACCATCGAAAGTTGATAA | 1168 |
| 1374 | Strongyloides spp | AGGTAATTCTTGAGCTAATACACGCT | 1163 | ATACCCTACCATCGAAAGTTGATAA | 1168 |
| 1375 | Strongyloides spp | AGGTAATTCTTGAGCTAATACACGCT | 1163 | AATACCCTACCATCGAAAGTTGATA | 1167 |
| 1376 | Strongyloides spp | GAGGTAATTCTTGAGCTAATACACGC | 1179 | CTACCATCGAAAGTTGATAAACCAG | 1170 |
| 1377 | Strongyloides spp | AGGTAATTCTTGAGCTAATACACGCTA | 1180 | CTACCATCGAAAGTTGATAAACCAG | 1170 |
| 1378 | Strongyloides spp | GAGGTAATTCTTGAGCTAATACACGC | 1179 | ATACCCTACCATCGAAAGTTGATAA | 1168 |
| 1379 | Strongyloides spp | GAGGTAATTCTTGAGCTAATACACGC | 1179 | AATACCCTACCATCGAAAGTTGATA | 1167 |
| 1380 | Strongyloides spp | AGGTAATTCTTGAGCTAATACACGCTA | 1180 | ATACCCTACCATCGAAAGTTGATAA | 1168 |
| 1381 | Strongyloides spp | AGGTAATTCTTGAGCTAATACACGCTA | 1180 | AATACCCTACCATCGAAAGTTGATA | 1167 |
| 1382 | Strongyloides spp | TGAGGTAATTCTTGAGCTAATACACG | 1161 | CCATCGAAAGTTGATAAACCAGATA | 1181 |
| 1383 | Strongyloides spp | AGGTAATTCTTGAGCTAATACACGCT | 1163 | CCATCGAAAGTTGATAAACCAGATA | 1181 |
| 1384 | Strongyloides spp | AGGTAATTCTTGAGCTAATACACGC | 1182 | AATACCCTACCATCGAAAGTTGATAA | 1162 |
| 1385 | Strongyloides spp | GGTAATTCTTGAGCTAATACACGCT | 1183 | AATACCCTACCATCGAAAGTTGATAA | 1162 |
| 1386 | Strongyloides spp | TGAGGTAATCTTGAGCTAATACACG | 1161 | TACCATCGAAAGTTGATAAACCAGA | 1184 |
| 1387 | Strongyloides spp | AGGTAATTCTTGAGCTAATACACGCT | 1163 | TACCATCGAAAGTTGATAAACCAGA | 1184 |
| 1388 | Strongyloides spp | AGGTAATTCTTGAGCTAATACACGC | 1182 | CATCGAAAGTTGATAAACCAGATACA | 1164 |
| 1389 | Strongyloides spp | GGTAATTCTTGAGCTAATACACGCT | 1183 | CATCGAAAGTTGATAAACCAGATACA | 1164 |
| 1390 | Strongyloides spp | TGAGGTAATTCTTGAGCTAATACACG | 1161 | ACCATCGAAAGTTGATAAACCAGAT | 1185 |
| 1391 | Strongyloides spp | AGGTAATTCTTGAGCTAATACACGCT | 1163 | ACCATCGAAAGTTGATAAACCAGAT | 1185 |
| 1392 | Strongyloides spp | GAGGTAATTCTTGAGCTAATACACGC | 1179 | CCATCGAAAGTTGATAAACCAGATA | 1181 |
| 1393 | Strongyloides spp | TGAGGTAATTCTTGAGCTAATACACG | 1161 | TCGAAAGTTGATAAACCAGATACAC | 1186 |
| 1394 | Strongyloides spp | AGGTAATTCTTGAGCTAATACACGC | 1182 | ATCGAAAGTTGATAAACCAGATACAC | 1187 |
| 1395 | Strongyloides spp | AGGTAATTCTTGAGCTAATACACGCTA | 1180 | CCATCGAAAGTTGATAAACCAGATA | 1181 |
| 1396 | Strongyloides spp | TGAGGTAATTCTTGAGCTAATACACG | 1161 | ATCGAAAGTTGATAAACCAGATACA | 1188 |
| 1397 | Strongyloides spp | TGAGGTAATTCTTGAGCTAATACACG | 1161 | CATCGAAAGTTGATAAACCAGATAC | 1189 |
| 1398 | Strongyloides spp | GAGGTAATTCTTGAGCTAATACACGC | 1179 | TACCATCGAAAGTTGATAAACCAGA | 1184 |
| 1399 | Strongyloides spp | AGGTAATTCTTGAGCTAATACACGC | 1182 | CCATCGAAAGTTGATAAACCAGATAC | 1190 |
| 1400 | Strongyloides spp | GGTAATTCTTGAGCTAATACACGCT | 1183 | CCATCGAAAGTTGATAAACCAGATAC | 1190 |
| 1401 | Taenia spp | CGTCTCAGTTCCACTTGATTCTATT | 1191 | AATATATAGTGTGTCGGGCATCAGT | 1192 |
| 1402 | Taenia spp | GGTATTGTCAACTTCTTCTGTACCTTC | 1193 | ATACCTAGTTCGCATGTACACTCCT | 1194 |
| 1403 | Taenia spp | GGTATTGTCAACTTCTTCTGTACCTTC | 1193 | TACCTAGTTCGCATGTACACTCCTC | 1195 |
| 1404 | Taenia spp | AAGGATCATTACACGTTCCTTCATT | 1196 | GAAGGTACAGAAGAAGTTGACAATACC | 1197 |
| 1405 | Taenia spp | GATCATTACACGTTCCTTCATTGTT | 1198 | GAAGGTACAGAAGAAGTTGACAATACC | 1197 |
| 1406 | Taenia spp | ATTGTCAACTTCTTCTGTACCTTCTTT | 1199 | ATACCTAGTTCGCATGTACACTCCT | 1194 |
| 1407 | Taenia spp | AGGAGTGTACATGCGAACTAGGTAT | 1200 | ACAGTTACTTCGTTACTCCTGCTCTC | 1201 |
| 1408 | Taenia spp | GAGGAGTGTACATGCGAACTAGGTAT | 1202 | ACACAGTTACTTCGTTACTCCTGCT | 1203 |
| 1409 | Taenia spp | AGGAGTGTACATGCGAACTAGGTAT | 1200 | ACACAGTTACTTCGTTACTCCTGCTC | 1204 |
| 1410 | Taenia spp | GGAGTGTACATGCGAACTAGGTATG | 1205 | ACAGTTACTTCGTTACTCCTGCTCTC | 1201 |
| 1411 | Taenia spp | AGGAGTGTACATGCGAACTAGGTAT | 1200 | CACAGTTACTTCGTTACTCCTGGTCT | 1206 |
| 1412 | Taenia spp | GAGTGTACATGCGAACTAGGTATGC | 1207 | ACAGTTACTTCGTTACTCCTGCTCTC | 1201 |
| 1413 | Taenia spp | GAGGAGTGTACATGCGAACTAGGTAT | 1202 | CACAGTTACTTCGTTACTCCTGCTC | 1208 |
| 1414 | Taenia spp | GAGGAGTGTACATGCGAACTAGGTAT | 1202 | ACAGTTACTTCGTTACTCCTGCTCT | 1209 |
| 1415 | Taenia spp | GAGGAGTGTACATGCGAACTAGGTAT | 1202 | CAGTTACTTCGTTACTCCTGCTCTC | 1210 |
| 1416 | Taenia spp | AGGAGTGTACATGCGAACTAGGTATG | 1211 | CAGTTACTTCGTTACTCCTGCTCTC | 1210 |
| 1417 | Taenia spp | TGTCAACTTCTTCTGTACCTTCTTTTC | 1212 | ATACCTAGTTCGCATGTACACTCCT | 1194 |
| 1418 | Taenia spp | CGGTATTGTCAACTTCTTCTGTACCT | 1213 | ATACCTAGTTCGCATGTACACTCCT | 1194 |
| 1419 | Taenia spp | GGTATTGTCAACTTCTTCTGTACCTT | 1214 | ATACCTAGTTCGCATGTACACTCCT | 1194 |
| 1420 | Taenia spp | CGGTATTGTCAACTTCTTCTGTACC | 1215 | ATACCTAGTTCGCATGTACACTCCTC | 1216 |
| 1421 | Taenia spp | CGGTATTGTCAACTTCTTCTGTACC | 1215 | TACCTAGTTCGCATGTACACTCCTCT | 1217 |
| 1422 | Taenia spp | CGGTATTGTCAACTTCTTCTGTACCT | 1213 | TACCTAGTTCGCATGTACACTCCTC | 1195 |
| 1423 | Taenia spp | CGGTATTGTCAACTTCTTCTGTACC | 1215 | CATACCTAGTTCGCATGTACACTCCT | 1218 |
| 1424 | Taenia spp | GATCATTACACGTTCCTTCATTGTT | 1198 | GAAAAGAAGGTACAGAAGAAGTTGACA | 1219 |
| 1425 | Taenia spp | GATCATTACACGTTCCTTCATTGTT | 1198 | AGGTACAGAAGAAGTTGACAATACCG | 1220 |
| 1426 | Taenia spp | GATCATTACACGTTCCTTCATTGTT | 1198 | AAGGTACAGAAGAAGTTGACAATACC | 1221 |
| 1427 | Taenia spp | CTTCTGTACCTTCTTTTCTTACCCTG | 1222 | ATACCTAGTTCGGATGTACACTCCTC | 1216 |
| 1428 | Taenia spp | AGGATCATTACACGTTCCTTCATT | 1223 | GAAGGTACAGAAGAAGTTGACAATACC | 1197 |
| 1429 | Taenia spp | AAGGATCATTACACGTTCCTTCAT | 1224 | GAAGGTACAGAAGAAGTTGACAATACC | 1197 |
| 1430 | Taenia spp | TTGTCAACTTCTTCTGTACCTTCTTTT | 1225 | ATACCTAGTTCGCATGTACACTCCT | 1194 |
| 1431 | Taenia spp | ATCATTACACGTTCCTTCATTGTTGT | 1226 | GGTACAGAAGAAGTTGACAATACCG | 1227 |
| 1432 | Taenia spp | CATTACACGTTCCTTCATTGTTTGT | 1228 | GAAGGTACAGAAGAAGTTGACAATACC | 1197 |
| 1433 | Taenia spp | ATTGTCAACTTCTTCTGTACCTTCTT | 1229 | ATACCTAGTTCGCATGTACACTCCT | 1194 |
| 1434 | Taenia spp | TTGTCAACTTCTTCTGTACCTTCTTTT | 1225 | TACCTAGTTCGCATGTACACTCCTC | 1195 |
| 1435 | Taenia spp | ATCATGTGTCGTCTTTGTGCTATAC | 1230 | ACAGTTACTTCGTTACTCCTGCTCTC | 1201 |
| 1436 | Taenia spp | GTCAACTTCTTCTGTACCTTCTTTTC | 1231 | ATACCTAGTTCGCATGTACACTCCT | 1194 |
| 1437 | Taenia spp | AGGAGTGTACATGCGAACTAGGTAT | 1200 | ACACAGTTACTTCGTTACTCCTGCT | 1203 |
| 1438 | Taenia spp | GGAGTGTACATGCGAACTAGGTATG | 1205 | ACACAGTTACTTCGTTACTCCTGGT | 1203 |
| 1439 | Taenia spp | GAGTGTACATGCGAACTAGGTATGC | 1207 | ACACAGTTACTTCGTTACTCCTGCT | 1203 |
| 1440 | Taenia spp | AGGAGTGTACATGCGAACTAGGTAT | 1200 | CACACAGTTACTTCGTTACTCCTGC | 1232 |
| 1441 | Taenia spp | GGAGTGTACATGCGAACTAGGTATG | 1205 | CACACAGTTACTTCGTTACTCCTGC | 1232 |
| 1442 | Taenia spp | GGAGTGTACATGCGAACTAGGTATG | 1205 | GCACACAGTTACTTCGTTACTCCTG | 1233 |
| 1443 | Taenia spp | GAGTGTACATGCGAACTAGGTATGC | 1207 | CACACAGTTACTTCGTTACTCCTGC | 1232 |
| 1444 | Taenia spp | GAGTGTACATGCGAACTAGGTATGC | 1207 | GCACACAGTTACTTCGTTACTCCTG | 1233 |
| 1445 | Taenia spp | AGGAGTGTACATGCGAACTAGGTAT | 1200 | CACAGTTACTTCGTTACTCCTGCTC | 1208 |
| 1446 | Taenia spp | AGGAGTGTACATGCGAACTAGGTAT | 1200 | ACAGTTACTTCGTTACTCGTGCTCT | 1209 |
| 1447 | Taenia spp | GGAGTGTACATGCGAACTAGGTATG | 1205 | CACAGTTACTTCGTTACTCCTGCTC | 1208 |
| 1448 | Taenia spp | GAGTGTACATGCGAACTAGGTATGC | 1207 | CACAGTTACTTCGTTACTCCTGCTC | 1208 |
| 1449 | Taenia spp | GGAGTGTACATGCGAACTAGGTATG | 1205 | ACAGTTACTTCGTTACTCCTGCTCT | 1209 |
| 1450 | Taenia spp | AGGAGTGTACATGCGAACTAGGTAT | 1200 | AGTTACTTCGTTACTCCTGCTCTCG | 1234 |
| 1451 | Toxocara spp. | CAGATTGAGAGCTCTTTCTTGATTC | 1235 | ATCACAGACCTGTTATTGCTCAACT | 1236 |
| 1452 | Toxocara spp. | CAGATTGAGAGCTCTTTCTTGATTC | 1235 | CTTGTCCCTCTAAGAAGTCTTCCG | 1237 |
| 1453 | Toxocara spp. | ACAGATTGAGAGCTCTTTCTTGATTC | 1238 | CTTGTCCCTCTAAGAAGTCTTCCG | 1237 |
| 1454 | Toxocara spp. | ACAGATTGAGAGCTCTTTCTTGATTC | 1238 | CCTCTAAGAAGTCTTCCGGACTTG | 1239 |
| 1455 | Toxocara spp. | CAGATTGAGAGCTCTTTCTTGATTC | 1235 | TTGTCCCTCTAAGAAGTCTTCCG | 1240 |
| 1456 | Toxocara spp. | ACAGATTGAGAGCTCTTTCTTGATT | 1241 | CTTGTCCCTCTAAGAAGTCTTCCG | 1237 |
| 1457 | Toxocara spp. | ACAGATTGAGAGCTCTTTCTTGATTC | 1238 | TTGTCCCTCTAAGAAGTCTTCCG | 1240 |
| 1458 | Toxocara spp. | CAGATTGAGAGCTCTTTCTTGATT | 1242 | CTTGTCCCTCTAAGAAGTCTTCCG | 1237 |
| 1459 | Toxocara spp. | CAGATTGAGAGCTCTTTCTTGATTC | 1235 | CCCTCTAAGAAGTCTTCCGGACT | 1243 |
| 1460 | Toxocara spp. | ACAGATTGAGAGCTCTTTCTTGATT | 1241 | CCTCTAAGAAGTCTTCCGGACTTG | 1239 |
| 1461 | Toxocara spp. | ACAGATTGAGAGCTCTTTCTTGATTC | 1238 | CCCTCTAAGAAGTCTTCCGGACT | 1243 |
| 1462 | Toxocara spp. | AGATTGAGAGCTCTTTCTTGATTCG | 1244 | CTTGTCCCTCTAAGAAGTCTTCCG | 1237 |
| 1463 | Toxocara spp. | CAGATTGAGAGCTCTTCTTGATTC | 1245 | CTTGTCCCTCTAAGAAGTCTTCCG | 1237 |
| 1464 | Toxocara spp. | ACAGATTGAGAGCTCTTTCTTGATT | 1241 | TTGTCCCTCTAAGAAGTCTTCCG | 1240 |
| 1465 | Toxocara spp. | CAGATTGAGAGCTCTTTCTTGATT | 1242 | TTGTCCCTCTAAGAAGTCTTCCG | 1240 |
| 1466 | Toxocara spp. | ACAGATTGAGAGCTCTTTCTTGATT | 1241 | CCCTCTAAGAAGTCTTCCGGACT | 1243 |
| 1467 | Toxocara spp. | CAGATTGAGAGCTCTTTCTTGATT | 1242 | CCCTCTAAGAAGTCTTCCGGACT | 1243 |
| 1468 | Toxocara spp. | AGATTGAGAGCTCTTTCTTGATTCG | 1244 | TTGTCCCTCTAAGAAGTCTTCCG | 1240 |
| 1469 | Toxocara spp. | AATATGGAGAAGTAAGATCGTGGC | 1246 | GATTTCTTGTGCATACATAGTCTTGG | 1247 |
| 1470 | Toxocara spp. | GAATATGGAGAAGTAAGATCGTGGC | 1248 | GATTTCTTGTGCATACATAGTCTTGG | 1247 |
| 1471 | Toxocara spp. | CAGATTGAGAGCTCTTCTTGATTC | 1245 | TTGTCCCTCTAAGAAGTCTTCCG | 1240 |
| 1472 | Toxocara spp. | AATATGGAGAAGTAAGATCGTGGC | 1246 | GACGTTACCTCCAACCTAGATTTTG | 1249 |
| 1473 | Toxocara spp. | AATATGGAGAAGTAAGATCGTGGC | 1246 | TGACGTTACCTCCAACCTAGATTTT | 1250 |
| 1474 | Toxocara spp. | GAATATGGAGAAGTAAGATCGTGGC | 1248 | GACGTTACCTCCAACCTAGATTTTG | 1249 |
| 1475 | Toxocara spp. | GAATATGGAGAAGTAAGATCGTGGC | 1248 | TGACGTTACCTCCAACCTAGATTTT | 1250 |
| 1476 | Toxocara spp. | AGATTGAGAGCTCTTCTTGATTCG | 1251 | CTTGTCCCTCTAAGAAGTCTTCCG | 1237 |
| 1477 | Toxocara spp. | GATTGAGAGCTCTTTCTTGATTCG | 1252 | CTTGTCCCTCTAAGAAGTCTTCCG | 1237 |
| 1478 | Toxocara spp. | AATATGGAGAAGTAAGATCGTGGC | 1246 | AGCGATTTCTTGTGCATACATAGTC | 1253 |
| 1479 | Toxocara spp. | AATATGGAGAAGTAAGATCGTGGC | 1246 | GCGATTTCTTGTGCATACATAGTCT | 1254 |
| 1480 | Toxocara spp. | GAATATGGAGAAGTAAGATCGTGGC | 1248 | GCGATTTCTTGTGCATACATAGTCT | 1254 |
| 1481 | Toxocara spp. | AGAATATGGAGAAGTAAGATCGTGG | 1255 | GATTTCTTGTGCATACATAGTCTTGG | 1247 |
| 1482 | Toxocara spp. | AATATGGAGAAGTAAGATCGTGGC | 1246 | CGATTTCTTGTGCATACATAGTCTT | 1256 |
| 1483 | Toxocara spp. | AATATGGAGAAGTAAGATCGTGGC | 1246 | ATTTCTTGTGCATACATAGTCTTGG | 1257 |
| 1484 | Toxocara spp. | GAATATGGAGAAGTAAGATCGTGGC | 1248 | CGATTTCTTGTGCATACATAGTCTT | 1256 |
| 1485 | Toxocara spp. | GAATATGGAGAAGTAAGATCGTGGC | 1248 | ATTTCTTGTGCATACATAGTCTTGG | 1257 |
| 1486 | Toxocara spp. | GAATATGGAGAAGTAAGATCGTGGC | 1248 | ATAGTCTTGGCAAGGTACGTGGTAT | 1258 |
| 1487 | Toxocara spp. | AATATGGAGAAGTAAGATCGTGGC | 1246 | TTTCTTGTGCATACATAGTCTTGGC | 1259 |
| 1488 | Toxocara spp. | CGTTTCAGAGTTGAGTAGACTTAATGA | 1260 | TATGTATTTGTATGTGTGTGTCGGC | 1261 |
| 1489 | Toxocara spp. | TCGTTTCAGAGTTGAGTAGACTTAATG | 1262 | TATGTATTTGTATGTGTGTGTCGGC | 1261 |
| 1490 | Toxocara spp. | AGAATATGGAGAAGTAAGATCGTGG | 1255 | GACGTTACCTCCAACCTAGATTTTG | 1249 |
| 1491 | Toxocara spp. | AATATGGAGAAGTAAGATCGTGGC | 1246 | ACGTTACCTCCAACCTAGATTTTG | 1263 |
| 1492 | Toxocara spp. | GAATATGGAGAAGTAAGATCGTGGC | 1248 | ACGTTACCTCCAACCTAGATTTTG | 1263 |
| 1493 | Toxocara spp. | AGATTGAGAGCTCTTCTTGATTCG | 1251 | TTGTCCCTCTAAGAAGTCTTCCG | 1240 |
| 1494 | Toxocara spp. | GAATATGGAGAAGTAAGATCGTGGC | 1248 | TTCTTGTGCATACATAGTCTTGGC | 1264 |
| 1495 | Toxocara spp. | AATATGGAGAAGTAAGATCGTGGC | 1246 | GCGATTTCTTGTGCATACATAGTC | 1265 |
| 1496 | Toxocara spp. | TCAGAATATGGAGAAGTAAGATCGTG | 1266 | GATTTCTTGTGCATACATAGTCTTGG | 1247 |
| 1497 | Toxocara spp. | AATATGGAGAAGTAAGATCGTGGC | 1246 | TTCTTGTGCATACATAGTCTTGGC | 1264 |
| 1498 | Toxocara spp. | AATATGGAGAAGTAAGATCGTGGC | 1246 | AGCGATTTCTTGTGCATACATAGT | 1267 |
| 1499 | Toxocara spp. | CAGAATATGGAGAAGTAAGATCGTG | 1268 | GATTTCTTGTGCATACATAGTCTTGG | 1247 |
| 1500 | Toxocara spp. | GATTGAGAGCTCTTTCTTGATTCG | 1252 | TTGTCCCTCTAAGAAGTCTTCCG | 1240 |
| 1501 | Toxoplasma gondii | CTGCTTACAATCGAAGACGGTAT | 1269 | TCAAAAAGAGAGTCAATTTCGATG | 1270 |
| 1502 | Toxoplasma gondii | GTTCTCTCTGTATGCGTGCATGT | 1271 | ATGTAAATAGAGCTCCGTAAATGCG | 1272 |
| 1503 | Toxoplasma gondii | GTTCTCTCTGTATGCGTGCATGT | 1271 | TGTAAATAGAGCTCCGTAAATGCG | 1273 |
| 1504 | Toxoplasma gondii | GTTCTCTCTGTATGCGTGCATGT | 1271 | GTAAATAGAGCTCCGTAAATGCG | 1274 |
| 1505 | Toxoplasma gondii | TCTTCCTCTCTTTGCCCCTTAGT | 1275 | CAAGAGCAAGAGAACAACTGAAGAA | 1276 |
| 1506 | Toxoplasma gondii | TCTTCCTCTCTTTGCCCCTTAGT | 1275 | CAAGAGCAAGAGAACAACTGAAGA | 1277 |
| 1507 | Toxoplasma gondii | TCTTCCTCTCTTTGCCCCTTAGT | 1275 | CAACAGCAAGAGAACAACTGAAGA | 1278 |
| 1508 | Toxoplasma gondii | TCTTCCTCTCTTTGCCCCTTAGT | 1275 | AAAGAGCAAGAGAACAACTGAAGAA | 1279 |
| 1509 | Toxoplasma gondii | TCTTCCTCTCTTTGCCCCTTAGT | 1275 | AGAGCAAGAGAACAACTGAAGAAGG | 1280 |
| 1510 | Toxoplasma gondii | TCTTCCTCTCTTTGCCCCTTAGT | 1275 | GAGCAAGAGAACAACTGAAGAAGG | 1281 |
| 1511 | Toxoplasma gondii | TCTTCCTCTCTTTGCCCCTTAGT | 1275 | AGCAAGAGAACAACTGAAGAAGGAG | 1282 |
| 1512 | Toxoplasma gondii | GATTTGTCGTGGATACTCCTTTATCTA | 1283 | GGAGAGAAAACACACAAAAGACAAT | 1284 |
| 1513 | Toxoplasma gondii | ATACTCCTTTATCTACGGTGTCTGTGT | 1285 | GGAGAGAAAACACACAAAAGACAAT | 1284 |
| 1514 | Toxoplasma gondii | ACTTTTGTTCTTTTTAAATGCATATCG | 1286 | AGTCACACACACAAATCTATGAGGA | 1287 |
| 1515 | Toxoplasma gondii | TCTTCCTCTCTTTGCCCCTTAGT | 1275 | GCAAGAGAACAACTGAAGAAGGAG | 1288 |
| 1516 | Toxoplasma gondii | TCTTCCTCTCTTTGCCCCTTAGT | 1275 | CAGCAAGAGAACAACTGAAGAAGG | 1289 |
| 1517 | Toxoplasma gondii | TCTTCCTCTCTTTGCCCCTTAGT | 1275 | AAGAGCAAGAGAACAACTGAAGAA | 1290 |
| 1518 | Toxoplasma gondii | TCTTCCTCTCTTTGCCCCTTAGT | 1275 | AGCAAGAGAACAACTGAAGAAGG | 1291 |
| 1519 | Toxoplasma gondii | TCTTCCTCTCTTTGCCCCTTAGT | 1275 | AAGAGAACAACTGAAGAAGGGGAG | 1292 |
| 1520 | Toxoplasma gondii | TCTTCCTCTCTTTGCCCCTTAGT | 1275 | AGCAAGAGAACAACTGAAGAAGGA | 1293 |
| 1521 | Toxoplasma gondii | TCTTCCTCTCTTTGCCCCTTAGT | 1275 | CAAGAGAACAACTGAAGAAGGAGA | 1294 |
| 1522 | Toxoplasma gondii | ACTTTTGTTCTTTTTAAATGCATATCG | 1286 | AACGAAAGTCACACACACAAATCTA | 1295 |
| 1523 | Toxoplasma gondii | AAATCAAAGTGTTTTGCAGTAGGAC | 1296 | AAGTCACACACACAAATCTATGAGGA | 1297 |
| 1524 | Toxoplasma gondii | TCCTTTATCTACGGTGTCTGTGTTAC | 1298 | GGAGAGAAAACACACAAAAGACAAT | 1284 |
| 1525 | Toxoplasma gondii | GTGGATACTCCTTTATCTACGGTGTC | 1299 | GGAGAGAAAACACACAAAAGACAAT | 1284 |
| 1526 | Toxoplasma gondii | GGATACTCCTTTATCTACGGTGTCTG | 1300 | GGAGAGAAAACACACAAAAGACAAT | 1284 |
| 1527 | Toxoplasma gondii | AAATCAAAGTGTTTTGCAGTAGGAC | 1296 | AAAGTCACACACACAAATCTATGAGG | 1301 |
| 1528 | Toxoplasma gondii | ATACTCCTTTATCTACGGTGTCTGTG | 1302 | GGAGAGAAAACACACAAAAGACAAT | 1284 |
| 1529 | Toxoplasma gondii | AAATCAAAGTGTTTTGCAGTAGGAC | 1296 | AGTCACACACACAAATCTATGAGGAA | 1303 |
| 1530 | Toxoplasma gondii | CTCCTTTATCTACGGTGTCTGTGTTA | 1304 | GGAGAGAAAACACACAAAAGACAAT | 1284 |
| 1531 | Toxoplasma gondii | TCACGGATCACTTCTAAATAGTTTCA | 1305 | GGAGAGAAAACACACAAAAGACAAT | 1284 |
| 1532 | Toxoplasma gondii | TACTCCTTTATCTACGGTGTCTGTGT | 1306 | GGAGAGAAAACACACAAAAGACAAT | 1284 |
| 1533 | Toxoplasma gondii | ACTCCTTTATCTACGGTGTCTGTGTT | 1307 | GGAGAGAAAACACACAAAAGACAAT | 1284 |
| 1534 | Toxoplasma gondii | CACGGATCACTTCTAAATAGTTTCAT | 1308 | GGAGAGAAAACACACAAAAGACAAT | 1284 |
| 1535 | Toxoplasma gondii | AAATCAAAGTGTTTTGCAGTAGGAC | 1296 | AACGAAAGTCACACACACAAATCTAT | 1309 |
| 1536 | Toxoplasma gondii | ATCAAAGTGTTTTGCAGTAGGACAT | 1310 | AACGAAAGTCACACACACAAATCTAT | 1309 |
| 1537 | Toxoplasma gondii | CAAAGTGTTTTGCAGTAGGACATCT | 1311 | AACGAAAGTCACACACACAAATCTAT | 1309 |
| 1538 | Toxoplasma gondii | TCTTCCTCTCTTTGCCCCTTAGT | 1275 | AGAGAACAACTGAAGAAGGGGAG | 1312 |
| 1539 | Toxoplasma gondii | TCTTCCTCTCTTTGCCCCTTAGT | 1275 | GCAAGAGAACAACTGAAGAAGGA | 1313 |
| 1540 | Toxoplasma gondii | CTTTTGTTCTTTTTAAATGCATATCG | 1314 | AGTCACACACACAAATCTATGAGGA | 1287 |
| 1541 | Toxoplasma gondii | CTTTTGTTCTTTTTAAATGCATATCG | 1314 | GTCACACACACAAATCTATGAGGAA | 1315 |
| 1542 | Toxoplasma gondii | CTTTTGTTCTTTTTAAATGCATATCG | 1314 | AAGTCACACACACAAATCTATGAGG | 1316 |
| 1543 | Toxoplasma gondii | TCTTCCTCTCTTTGCCCCTTAGT | 1275 | AACAGCAAGAGAACAACTGAAGAA | 1317 |
| 1544 | Toxoplasma gondii | CTTTTGTTCTTTTTAAATGCATATCG | 1314 | TCACACACACAAATCTATGAGGAAC | 1318 |
| 1545 | Toxoplasma gondii | CTTTTGTTCTTTTTAAATGCATATCG | 1314 | CACACACAAATCTATGAGGAACAGA | 1319 |
| 1546 | Toxoplasma gondii | CTTTTGTTCTTTTTAAATGCATATCG | 1314 | ACACACACAAATCTATGAGGAACAG | 1320 |
| 1547 | Toxoplasma gondii | AGTTTTGTTCTTTTTAAATGCATATCG | 1321 | AGTCACACACACAAATCTATGAGGA | 1287 |
| 1548 | Toxoplasma gondii | TCGTGGATACTCCTTTATCTACGGT | 1322 | GGAGAGAAAACACACAAAAGACAAT | 1284 |
| 1549 | Toxoplasma gondii | CAAATCAAAGTGTTTTTGCAGTAAGA | 1323 | AAAGTCACACACACAAATCTATGAGG | 1301 |
| 1550 | Toxoplasma gondii | CAAATCAAAGTGTTTTGCAGTAAGA | 1323 | GTCACACACACAAATCTATGAGGAAC | 1324 |
| 1551 | West Nile virus | CATGGCAATTATTCAACACAGATAG | 1325 | AGATCCATAAACCATTCTCGGTG | 1326 |
| 1552 | West Nile virus | CAGGCTGGAAGATTCAGTATAACTC | 1327 | AGATCCATAAACCATTCTCGGTG | 1326 |
| 1553 | West Nile virus | AGGCTGGAAGATTCAGTATAACTCC | 1328 | AGATCCATAAACCATTCTCGGTG | 1326 |
| 1554 | West Nile virus | ATGGCAATTATTCAACACAGATAGG | 1329 | AGATCCATAAACCATTCTCGGTG | 1326 |
| 1555 | West Nile virus | GAATCTCATGGCAATTATTCAACAC | 1330 | AGATCCATAAACCATTCTCGGTG | 1326 |
| 1556 | West Nile virus | ATCTCATGGCAATTATTCAACACAG | 1331 | AGATCCATAAACCATTCTCGGTG | 1326 |
| 1557 | West Nile virus | CTCATGGCAATTATTCAACACAGAT | 1332 | AGATCCATAAACCATTCTCGGTG | 1326 |
| 1558 | West Nile virus | TTGAATCTCATGGCAATTATTCAAC | 1333 | AGATCCATAAACCATTCTCGGTG | 1326 |
| 1559 | West Nile virus | AATCTCATGGCAATTATTCAACACA | 1334 | AGATCCATAAACCATTCTCGGTG | 1326 |
| 1560 | West Nile virus | CATGGCAATTATTTAACACAGATAGG | 1335 | AGATCCATAAACCATTCTCGGTG | 1326 |
| 1561 | West Nile virus | TCTCATGGCAATTATTTAACACAGAT | 1336 | AGATCCATAAACCATTCTCGGTG | 1326 |
| 1562 | West Nile virus | CTGGAAGATTCAGCATAACTCCAT | 1337 | AGATCCATAAACCATTCTCGGTG | 1326 |
| 1563 | West Nile virus | TGGAAGATTCAGCATAACTCCATC | 1338 | AGATCCATAAACCATTCTCGGTG | 1326 |
| 1564 | West Nile virus | TCTCATGGCAATTATTTAACACAGATAG | 1339 | GACATAACGTAATAGGCGCTAGTGT | 1340 |
| 1565 | West Nile virus | GCTGGAAGATTCAGTATAACACCATC | 1341 | AGATCCATAAACCATTCTCGGTG | 1326 |
| 1566 | West Nile virus | CTCATGGTAATTATTCAACACAGATAGG | 1342 | GCACCAACTGACATAACGTAATAGG | 1343 |
| 1567 | West Nile virus | TCTCATGGTAATTATTCAACACAGATAG | 1344 | GCACGAACTGACATAACGTAATAGG | 1343 |
| 1568 | West Nile virus | CTCATGGTAATTATTCAACACAGATAGG | 1342 | GACATAACGTAATAGGCGCTAGTGT | 1340 |
| 1569 | West Nile virus | TCTCATGGTAATTATTCAACACAGATAG | 1344 | GACATAACGTAATAGGCGCTAGTGT | 1340 |
| 1570 | West Nile virus | GTTGAATCTCATGGTAATTATTCAACAC | 1345 | GCACCAACTGACATAACGTAATAGG | 1343 |
| 1571 | West Nile virus | TGAATCTCATGGTAATTATTCAACACAG | 1346 | GCACCAACTGACATAACGTAATAGG | 1343 |
| 1572 | West Nile virus | CATGGCAATTATCCAACACAGATAG | 1347 | AGATCCATAAACCATTCTCGGTG | 1326 |
| 1573 | West Nile virus | TTGAATCTCATGGTAATTATTCAACACA | 1348 | GACATAACGTAATAGGCGCTAGTGT | 1340 |
| 1574 | West Nile virus | CAATTATTCAACACAGATAGGGGC | 1349 | AGATCCATAAACCATTCTCGGTG | 1326 |
| 1575 | West Nile virus | AAGATTCAGTATAACTCCATCGGC | 1350 | AGATCCATAAACCATTCTCGGTG | 1326 |
| 1576 | West Nile virus | TGAATCTCATGGCAATTATTCAAC | 1351 | AGATCCATAAACCATTCTCGGTG | 1326 |
| 1577 | West Nile virus | GAATCTCATGGCAATTATTCAACA | 1352 | AGATCCATAAACCATTCTCGGTG | 1326 |
| 1578 | West Nile virus | TGGCAATTATTCAACACAGATAGG | 1353 | AGATCCATAAACCATTCTCGGTG | 1326 |
| 1579 | West Nile virus | CCAGGCTGGAAGATTCAGTATAAC | 1354 | AGATCCATAAACCATTCTCGGTG | 1326 |
| 1580 | West Nile virus | ATCTCATGGCAATTATTCAACACA | 1355 | AGATCCATAAACCATTCTCGGTG | 1326 |
| 1581 | West Nile virus | TCATGGCAATTATTCAACACAGAT | 1356 | AGATCCATAAACCATTCTCGGTG | 1326 |
| 1582 | West Nile virus | AATCTCATGGCAATTATTCAACAC | 1357 | AGATCCATAAACCATTCTCGGTG | 1326 |
| 1583 | West Nile virus | GGCAATTATTCAACACAGATAGGG | 1358 | AGATCCATAAACCATTCTCGGTG | 1326 |
| 1584 | West Nile virus | TCTCATGGCAATTATTCAACACAG | 1359 | AGATCCATAAACCATTCTCGGTG | 1326 |
| 1585 | West Nile virus | TTGAATCTCATGGCAATTATTCAAC | 1333 | CAGATCCATAAACCATTCTCGGT | 1360 |
| 1586 | West Nile virus | TCATGGCAATTATTCAACACAGATAG | 1361 | GCACCAACTGACATAACGTAATAGG | 1343 |
| 1587 | West Nile virus | CTCATGGCAATTATTCAACACAGATA | 1362 | GCACCAACTGACATAACGTAATAGG | 1343 |
| 1588 | West Nile virus | AATTATTTAACACAGATAGGGGCCA | 1363 | AGATCCATAAACCATTCTCGGTG | 1326 |
| 1589 | West Nile virus | TCATGGCAATTATTCAACACAGATAG | 1361 | GACATAACGTAATAGGCGCTAGTGT | 1340 |
| 1590 | West Nile virus | CTCATGGCAATTATTCAACAGAGATA | 1362 | GACATAACGTAATAGGCGCTAGTGT | 1340 |
| 1591 | West Nile virus | CATGGCAATTACTCAACACAGATAG | 1364 | AGATCCATAAACCATTCTCGGTG | 1326 |
| 1592 | West Nile virus | TGGCAATTATTTAACACAGATAGGG | 1365 | AGATCCATAAACCATTCTCGGTG | 1326 |
| 1593 | West Nile virus | ATGGCAATTACTCAACACAGATAGG | 1366 | AGATCCATAAACCATTCTCGGTG | 1326 |
| 1594 | West Nile virus | GAATCTCATGGCAATTACTCAACAC | 1367 | AGATCCATAAACCATTCTCGGTG | 1326 |
| 1595 | West Nile virus | GCAATTATTTAACACAGATAGGGGC | 1368 | AGATCCATAAACCATTCTCGGTG | 1326 |
| 1596 | West Nile virus | AATCTCATGGCAATTATTTAACACA | 1369 | AGATCCATAAACCATTCTCGGTG | 1326 |
| 1597 | West Nile virus | TGGAAGATTCAGCATAACTCCAT | 1370 | AGATCCATAAACCATTCTCGGTG | 1326 |
| 1598 | West Nile virus | GAAGATTCAGCATAACTCCATCG | 1371 | AGATCCATAAACCATTCTCGGTG | 1326 |
| 1599 | West Nile virus | AGGCTGGAAGATTCAGCATAACT | 1372 | AGATCCATAAACCATTCTCGGTG | 1326 |
| 1600 | West Nile virus | GGAAGATTCAGCATAACTCCATC | 1373 | AGATCCATAAACCATTCTCGGTG | 1326 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a mixture, wherein said mixture comprises at least one primer pair selected from the group consisting of primer pair numbers 951, 1001, 151, 701, 1051, 351, 801, 101, and 1101, wherein primer pair number 951 amplifies a sequence present in a feline immunodeficiency virus, wherein primer pair number 1001 amplifies a sequence present in a feline leukemia virus, wherein primer pair number 151 amplifies a sequence present in a Borrelia burgdorferi organism, wherein primer pair number 701 amplifies a sequence present in a Dirofilaria immitis organism, wherein primer pair number 1051 amplifies a sequence present in a feline parvovirus, wherein primer pair number 351 amplifies a sequence present in a canine distemper virus, wherein primer pair number 801 amplifies a sequence present in a Ehrlichia canis organism, wherein primer pair number 101 amplifies a sequence present in a Bordetella organism, and wherein primer pair number 1101 amplifies a sequence present in a Giardia organism.

2. The composition of claim 1, wherein said mixture is a solid.

3. The composition of claim 1, wherein said mixture is a liquid.

4. The composition of claim 1, wherein said mixture comprises primer pair number 951.

5. The composition of claim 1, wherein said mixture comprises primer pair number 1001.

6. The composition of claim 1, wherein said mixture comprises primer pair number 151.

7. An article of manufacture comprising (a) a substrate defining a microfluidic chamber and (b) a mixture comprising at least one primer pair selected from the group consisting of primer pair numbers 951, 1001, 151, 701, 1051, 351, 801, 101, and 1101; wherein said mixture is within said chamber; wherein primer pair number 951 is capable of amplifying, within said chamber, a sequence present in a feline immunodeficiency virus; wherein primer pair number 1001 is capable of amplifying, within said chamber, a sequence present in a feline leukemia virus; wherein primer pair number 151 is capable of amplifying, within said chamber, a sequence present in a Borrelia burgdorferi organism; wherein primer pair number 701 is capable of amplifying, within said chamber, a sequence present in a Dirofilaria immitis organism; wherein primer pair number 1051 is capable of amplifying, within said chamber, a sequence present in a feline parvovirus; wherein primer pair number 351 is capable of amplifying, within said chamber, a sequence present in a canine distemper virus; wherein primer pair number 801 is capable of amplifying, within said chamber, a sequence present in a Ehrlichia canis organism; wherein primer pair number 101 is capable of amplifying, within said chamber, a sequence present in a Bordetella organism; and wherein primer pair number 1101 is capable of amplifying, within said chamber, a sequence present in a Giardia organism.

8. The article of manufacture of claim 7, wherein said mixture is a solid.

9. The article of manufacture of claim 7, wherein said mixture is a liquid.

10. The article of manufacture of claim 7, wherein said mixture comprises primer pair number 951.

11. The article of manufacture of claim 7, wherein said mixture comprises primer pair number 1001.

12. The article of manufacture of claim 7, wherein said mixture comprises primer pair number 151.

13. A diagnostic card for determining whether or not a cat contains any pathogen selected from the group consisting of feline immunodeficiency virus, feline leukemia virus, and Borrelia burgdorferi; wherein said card comprises a plurality of microfluidic chambers; wherein at least one of said microfluidic chambers comprises the primers of primer pair number 951, which is capable of amplifying, within said chamber, a sequence present in a feline immunodeficiency virus; wherein at least one of said microfluidic chambers comprises the primers of primer pair number 1001, which is capable of amplifying, within said chamber, a sequence present in a feline leukemia virus; and wherein at least one of said microfluidic chambers comprises the primers of primer pair number 151, which is capable of amplifying, within said chamber, a sequence present in a Borrelia burgdorferi organism.

14. A diagnostic card for determining whether or not a dog contains any pathogen selected from the group consisting of Borrelia burgdorferi, Dirofilaria immitis, and canine distemper virus; wherein said card comprises a plurality of microfluidic chambers; wherein at least one of said microfluidic chambers comprises the primers of primer pair number 151, which is capable of amplifying, within said chamber, a sequence present in a Borrelia burgdorferi organism; wherein at least one of said microfluidic chambers comprises the primers of primer pair number 701, which is capable of amplifying, within said chamber, a sequence present in a Dirofilaria immitis organism; and wherein at least one of said microfluidic chambers comprises the primers of primer pair number 351, which is capable of amplifying, within said chamber, a sequence present in a canine distemper virus.

15. A method for determining whether or not a mammal contains a pathogen, wherein said method comprises performing an amplification reaction with a primer pair selected from the group consisting of primer pair numbers 951, 1001, 151, 701, 1051, 351, 801, 101, and 1101 to determine whether or not a sample from said mammal contains nucleic acid capable of being amplified with said primer pair, wherein the presence of said nucleic acid indicates that said mammal contains said pathogen.

16. The method of claim 15, wherein said mammal is a cat.

17. The method of claim 15, wherein said mammal is a dog.

18. The method of claim 15, wherein said sample is a blood sample.

19. The method of claim 15, wherein said primer pair is primer pair number 951, and wherein the presence of said nucleic acid indicates that said mammal contains a feline immunodeficiency virus.

20. The method of claim 15, wherein said primer pair is primer pair number 1001, and wherein the presence of said nucleic acid indicates that said mammal contains a feline leukemia virus.

21. The method of claim 15, wherein said primer pair is primer pair number 151, and wherein the presence of said nucleic acid indicates that said mammal contains a Borrelia burgdorferi organism.

22. A method for making an article of manufacture for determining whether or not a mammal contains a pathogen, said method comprising:
(a) providing a substrate defining a microfluidic chamber, and (b) placing a mixture into said chamber to form said article of manufacture, wherein said mixture comprises at least one primer pair selected from the group consisting of primer pair numbers 951,1001, 151, 701, 1051, 351, 801,101, and 1101; wherein said mixture is within said chamber; wherein primer pair number 951 is capable of amplifying, within said chamber, a sequence present in a feline immunodeficiency virus; wherein primer pair number 1001 is capable of amplifying, within said chamber, a sequence present in a feline leukemia virus; wherein primer pair number 151 is capable of amplifying, within said chamber, a sequence present in a Borrelia burgdorferi organism; wherein primer pair number 701 is capable of amplifying, within said chamber, a sequence present in a Dirofilaria immitis organism; wherein primer pair number 1051 is capable of amplifying, within said chamber, a sequence present in a feline parvovirus; wherein primer pair number 351 is capable of amplifying, within said chamber, a sequence present in a canine distemper virus; wherein primer pair number 801 is capable of amplifying, within said chamber, a sequence present in a Ehrlichia canis organism; wherein primer pair number 101 is capable of amplifying, within said chamber, a sequence present in a Bordetella organism; and wherein primer pair number 1101 is capable of amplifying, within said chamber, a sequence present in a Giardia organism.

23. The method of claim 22, wherein said mixture is a solid.

24. The method of claim 22, wherein said mixture is a liquid.

25. The method of claim 22, wherein said mammal is a cat.

26. The method of claim 22, wherein said mammal is a dog.

27. The method of claim 22, wherein said primer pair is primer pair number 951, and wherein the presence of said nucleic acid indicates that said mammal contains a feline immunodeficiency virus.

28. The method of claim 22, wherein said primer pair is primer pair number 1001, and wherein the presence of said nucleic acid indicates that said mammal contains a feline leukemia virus.

29. The method of claim 22, wherein said primer pair is primer pair number 151, and wherein the presence of said nucleic acid indicates that said mammal contains a Borrelia burgdorferi organism.
